(19) United States
(12) Patent Application Publication
(10) Pub. No.: US 2023/0172943 A1
(43) Pub. Date: Jun. 8, 2023

(54) ORAL SOLID MELOXICAM FORMULATIONS FOR THE TREATMENT OF ACUTE PAIN

(71) Applicant: Mylan Specialty L.P., Morgantown, WV (US)

(72) Inventors: Richard Allan, Broadstairs (GB); Amit Antarkar, Hyderabad (IN); Santanu Chakraborty, Hyderabad (IN); Abhijit Deshmukh, Hyderabad (IN); Matthew A. Hummel, Morgantown, WV (US); Ashish Jaiswal, Hyderabad (IN); Akhilesh Dixit, Hyderabad (IN); Ritesh Kakaria, Hyderabad (IN); Pankaj Patil, Hyderabad (IN); Russ Rackley, Morgantown, WV (US); Andrew Shaw, Morgantown, WV (US); Jeffrey P. Smith, Morgantown, WV (US)

(73) Assignee: Mylan Specialty L.P., Morgantown, WV (US)

(21) Appl. No.: 17/920,233

(22) PCT Filed: Apr. 20, 2021
(86) PCT No.: PCT/US2021/028150

§ 371 Date: Oct. 20, 2022

(30) Foreign Application Priority Data
Apr. 21, 2020 (IN) 202031017033

(51) Int. Cl. A61K 31/5415; A61K 31/60; A61P 29/02; A61K 9/20

(52) U.S. Cl. CPC A61K 31/5415; A61K 31/60; A61P 29/02; A61K 9/2054

(57) ABSTRACT

Oral solid pharmaceutical compositions containing meloxicam co-crystals described herein allow for the use of meloxicam for the treatment of acute pain. In particular, the improved dissolution and bioavailability of the meloxicam co-crystal compositions provide more rapid uptake of meloxicam in vivo as evidenced by increase blood plasma concentrations in a decreased amount of time following administration. A correlation between improved plasma concentrations and in vivo analgesic action are provided for the first time.

### CROSS REFERENCE TO RELATED APPLICATION(S)

The present application claims priority to Indian Patent Application No. 202031017033, filed on Apr. 21, 2020, which is incorporated herein by reference in its entirety.

### FIELD OF THE INVENTION

The present invention relates to pharmaceutical compositions comprising meloxicam co-crystals, processes for preparing, and therapeutic uses of the same.

### BACKGROUND OF THE INVENTION

Meloxicam is a nonsteroidal anti-inflammatory drug in BCS class II. Meloxicam is known as (4-hydroxy-2-methyl-N-(5-methyl-2-thiazolyl)-2H-1,2-benzothiazine-3-carboxamide-1,1-dioxide). Meloxicam is depicted by the following chemical structure:

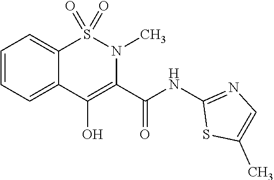 [SMILES: Cc1cnc(NC(=O)C2=C(O)c3ccccc3S(=O)(=O)N2C)s1]

The solubility of meloxicam varies depending upon pH and solvent polarity due to interconversion between ionization states. Because of its low solubility under acidic conditions, orally delivered meloxicam exhibits a Tₘₐₓ (time to reach maximum concentration) of 4-6 hours in humans and requires approximately 2-3 hours to reach the therapeutic concentration that enables the onset of action. Such slow onset prevents meloxicam from its potential application for the relief of mild-to-medium-level acute pain.

Several solutions have been suggested to enhance the low aqueous solubility of meloxicam. Examples of such proposals include, different crystalline polymorphic forms (U.S. Pat. No. 6,967,248); meloxicam salts of an inorganic or organic base (WO1999/049867); co-milling, co-grinding or co-kneading meloxicam in the presence of a cyclodextrin (U.S. Pat. No. 6,284,269); co-crystals of meloxicam and a co-crystal former (WO2009/094155); and surface-stabilized meloxicam nanoparticles having an effective average particle size of less than about 2000 nm (WO2005/002542). However, further improvements remain necessary to improve the dissolution and bioavailability of a poorly soluble drug.

As discussed in a Cochrane Review of meloxicam and a general review of NSAIDs for the treatment of acute pain, the currently available oral formulations of meloxicam (e.g., MOBIC tablets and VIVLODEX capsules) are not indicated for, nor are they suitable for, the treatment of acute pain due to the delayed absorption of the product (see Moore et al. Single dose oral meloxicam for acute postoperative pain in adults. Cochrane Database Syst Rev, 2009(4): p. CD007552; and Moore et al., Single dose oral analgesics for acute postoperative pain in adults—an overview of Cochrane reviews. Cochrane Database Syst Rev, 2015(9): p. CD008659). Meloxicam, however, has demonstrated efficacy in a number of clinical studies using intramuscular (IM) and intravenous (IV) formulations, including short-term studies in dental extraction and post bunionectomy surgery. Furthermore, some data are suggestive that IM meloxicam has a more rapid onset than oral meloxicam in rheumatoid arthritis pain and sciatica. See, e.g., Combe et al., Comparison of intramuscular and oral meloxicam in rheumatoid arthritis patients. Inflamm Res, 2001. 50 Suppl 1: p. S10-6; and Auvinet et al., Comparison of the onset and intensity of action of intramuscular meloxicam and oral meloxicam in patients with acute sciatica. Clin Ther, 1995. 17(6): p. 1078-98.

To date, the pharmacokinetics of certain meloxicam co-crystals have only been examined in rats through the administration of an oral gavage solution of relatively coarsely micronized co-crystals. See, for example, U.S. Pat. Nos. 8,124,603; 8,389,512; Cheney et al., Coformer Selection in Pharmaceutical Cocrystal Development: a Case Study of a Meloxicam Aspirin Cocrystal That Exhibits Enhanced Solubility and Pharmacokinetics, J. Pharma. Sci. 2011,100(6), 2172-2181; and Weyna et al., Improving Solubility and Pharmacokinetics of Meloxicam via Multiple-Component Crystal Formation, Mol. Pharmaceutics 2012, 9, 2094-2102. However, further improvements remain necessary to achieve suitable availability of meloxicam in a solid oral dosage form for the treatment of pain in humans.

The dental surgical pain model has been in widespread use for over 50 years, is well characterized, and is frequently used to investigate the pharmacodynamic properties of analgesic molecules (onset/offset, dose response, and potency). Efficacy in the dental model is highly predictive of efficacy in later stage models and has a high assay sensitivity (see Singla et al., A comparison of the clinical and experimental characteristics of four acute surgical pain models: dental extraction, bunionectomy, joint replacement, and soft tissue surgery. Pain, 2014. 155(3): p. 441-56). The model is also ideal as the surgery, the anesthetic regimen, and perioperative care protocols can be standardized across patients. The dental surgical pain model has demonstrated the efficacy of analgesic medications across a range of different pharmacology, including NSAIDs and opioids and specifically has demonstrated efficacy for an IV formulation of meloxicam recently approved as ANJESO (see, Singla, supra; and Christensen et al., A Randomized Double-Blind Controlled Trial of Intravenous Meloxicam in the Treatment of Pain Following Dental Impaction Surgery. J. Clin. Pharmacol., 2018. 58(5): p. 593-605).

### SUMMARY OF THE INVENTION

As described herein, applicants have for the first time identified that the oral solid dosage forms described herein can provide for the treatment of acute pain through achieving suitable in vivo meloxicam blood concentrations in a faster time frame versus commercially available oral solid meloxicam formulations.

Accordingly, in one aspect, a method for treating acute pain is provided comprising administering to a person in need of such treatment an oral solid pharmaceutical composition comprising a meloxicam co-crystal as described according to any embodiment here. In certain embodiments thereof, the oral solid compositions provide for a reduction of rescue medication, a reduced mean time of first perceptible relief, and/or a reduced mean time of meaningful relief as compared to MOBIC tablets as measured in a post-surgical dental pain study.

### DESCRIPTION OF THE FIGURES

FIG. 1a shows dissolution of meloxicam: succinic acid co-crystal formulations (4, micronized) and (1, nanosized) versus MOBIC tablets and VIVLODEX capsules as a function of time in 900 mL of 0.1N HCl.

FIG. 1b shows dissolution of meloxicam: succinic acid co-crystal formulations (1) and (4) versus MOBIC tablets and VIVLODEX capsules as a function of time in in 900 mL of acetate buffer (pH 4.5).

FIG. 1c shows dissolution of meloxicam: succinic acid co-crystal formulations (1) and (4) versus MOBIC tablets and VIVLODEX capsules as a function of time in 900 mL of phosphate buffer (pH 6.1).

FIG. 2a shows dissolution of meloxicam: xinafoic acid co-crystal formulations (5, micronized) and (2, nanosized) versus MOBIC tablets and VIVLODEX capsules as a function of time in 900 mL of 0.1N HCl, USP apparatus II, 37+/−2° C., and 75 rpm.

FIG. 2b shows dissolution of meloxicam: xinafoic acid co-crystal formulations (2) and (5) versus MOBIC tablets and VIVLODEX capsules as a function of time in 900 mL of acetate buffer (pH 4.5), USP apparatus II, 37+/−2° C., and 75 rpm.

FIG. 2c shows dissolution of meloxicam: xinafoic acid co-crystal formulations (2) and (5) versus MOBIC tablets and VIVLODEX capsules as a function of time in 900 mL of phosphate buffer (pH 6.1), USP apparatus II, 37+/−2° C., and 75 rpm.

FIG. 3a shows dissolution of meloxicam: salicylic acid co-crystal formulations (6, micronized) and (3, nanosized) versus MOBIC tablets and VIVLODEX capsules as a function of time in 900 mL of 0.1N HCl, USP apparatus II, 37+/−2° C., and 75 rpm.

FIG. 3b shows dissolution of meloxicam: salicylic acid co-crystal formulations (3) and (6) versus MOBIC tablets and VIVLODEX capsules as a function of time in 900 mL of acetate buffer (pH 4.5), USP apparatus II, 37+/−2° C., and 75 rpm.

FIG. 3c shows dissolution of meloxicam: salicylic acid co-crystal formulations (3) and (6) versus MOBIC tablets and VIVLODEX capsules as a function of time in 900 mL of phosphate buffer (pH 6.1), USP apparatus II, 37+/−2° C., and 75 rpm.

FIG. 4a shows the mean plasma concentration as a function of time as measured in the study of Example 8 for succinic acid formulations (1, filled diamonds) and (4, filled squares) as compared to MOBIC tablets (open triangles) for the first 24 hours after dosing.

FIG. 4b shows the mean plasma concentration as a function of time as measured in the study of Example 8 for xinafoic acid formulations (2, filled diamonds) and (5, filled squares) as compared to MOBIC tablets (open triangles) for the first 24 hours after dosing.

FIG. 4c shows the mean plasma concentration as a function of time as measured in the study of Example 8 for salicylic acid formulations (3, filled diamonds) and (6, filled squares) as compared to MOBIC tablets (open triangles) for the first 24 hours after dosing.

FIG. 5a shows the mean partial area under the curve [pAUC(0-t)] for succinic acid formulations (1, filled diamonds) and (4, filled squares) and MOBIC tablets (open triangles) for the first 8 hours after dosing as measured in the study of Example 8.

FIG. 5b shows the mean partial area under the curve [pAUC(0-t)] for xinafoic acid formulations (2, filled diamonds) and (5, filled squares) and MOBIC tablets (open triangles) for the first 8 hours after dosing as measured in the study of Example 8.

FIG. 5c shows the mean partial area under the curve [pAUC(0-t)] for salicylic acid formulations (3, filled diamonds) and (6, filled squares) and MOBIC tablets (open triangles) for the first 8 hours after dosing as measured in the study of Example 8.

FIG. 6 shows the mean plasma concentration of meloxicam for formulation (3) as compared to MOBIC tablets under fed and fasting conditions as measured in the study of Example 9 for the first 12 hours after dosing; filled squares: formulation (3, fasting); filled diamonds: formulation (3, fed); open triangles: MOBIC tablets (fasting); open circles: MOBIC tablets (fed).

FIG. 7 shows the mean partial area under the curve [pAUC(0-t)] for formulation (3) and MOBIC tablets for the first 8 hours after dosing under fed and fasting conditions as measured in the study of Example 9 for the first 8 hours after dosing; filled squares: formulation (3, fasting); filled diamonds: formulation (3, fed); open triangles: (MOBIC tablets, fasting); open circles: (MOBIC tablets, fed).

FIG. 8 shows a comparison of the mean blood plasma meloxicam concentration for the first 24 hours after dosing under fed and fasting conditions for formulation (3, Example 9) versus VIVLODEX capsules as reported in the US FDA Summary Basis of Approval (SBOA); filled squares: formulation (3, fasting); filled diamonds: formulation (3, fed); open triangles: (VIVLODEX caps, fasting); open circles: (VIVLODEX caps, fed).

FIG. 9 shows mean plasma concentrations of meloxicam for dose-proportional 5 mg and 10 mg formulations as projected based on the mean plasma concentration achieved with the 15 mg meloxicam formulation (3) of Example 9 under fasting conditions; filled squares, 15 mg dosage; filled triangles, 10 mg dosage; filled diamonds, 5 mg dosage; open circles, MOBIC tablets, 15 mg under fasting conditions from Example 9; the dotted line represents a blood plasma concentration of 1090 ng/mL.

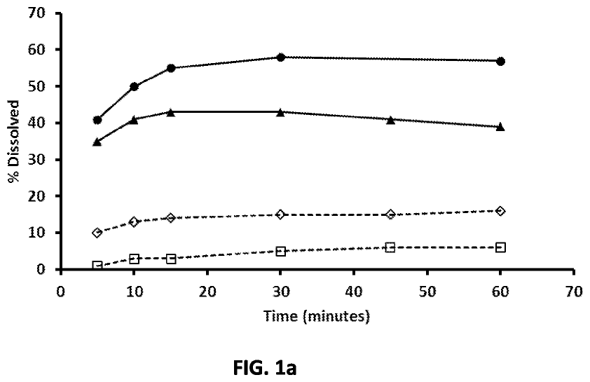

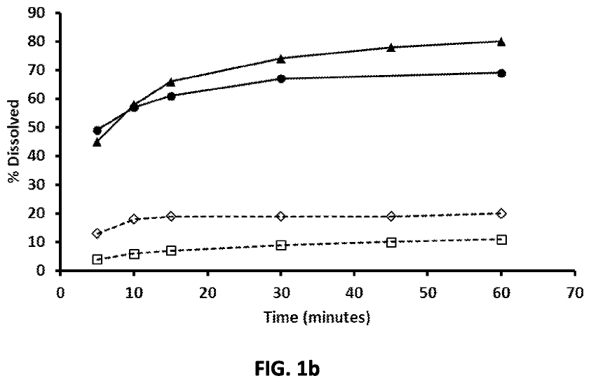

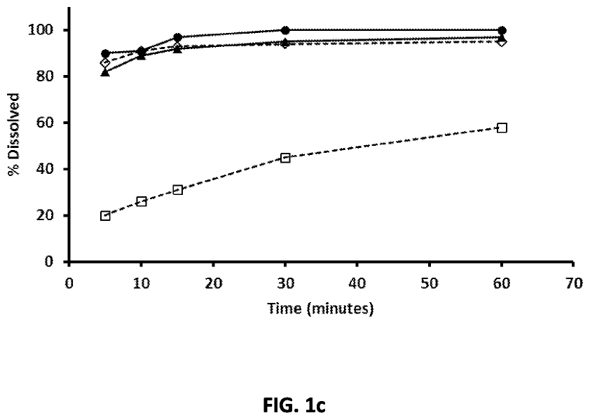

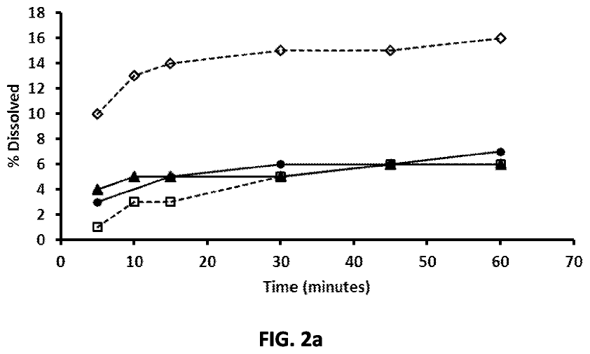

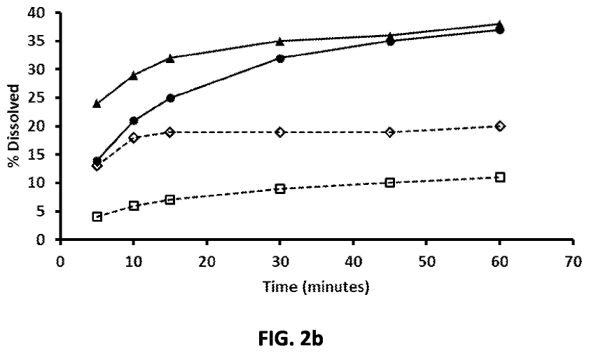

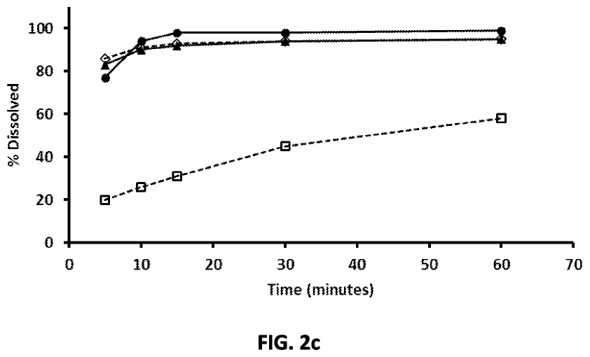

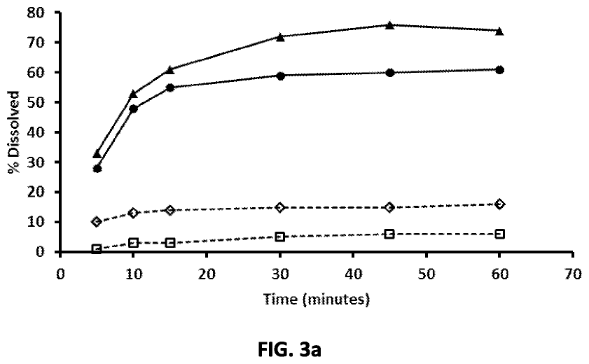

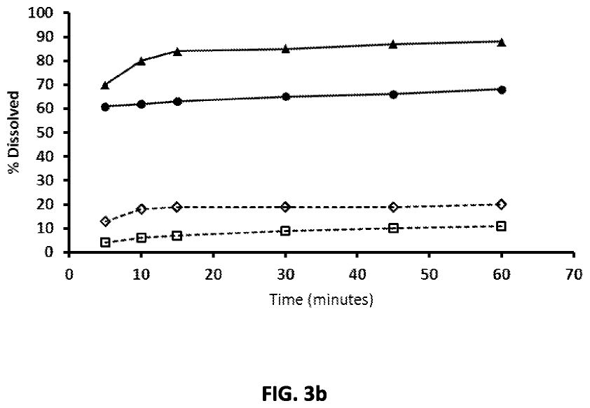

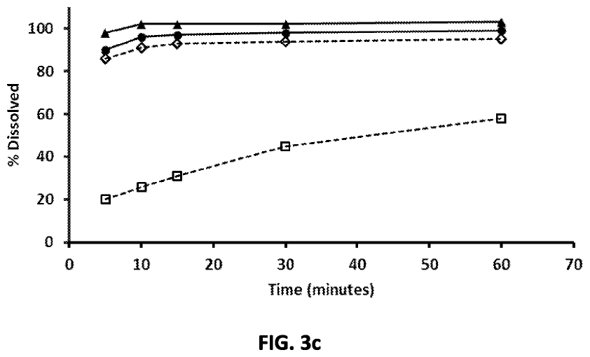

In each of FIGS. 1-3a-c, open squares represent MOBIC tablets, 15 mg; open diamonds represent VIVLODEX capsules, 15 mg; filled triangles represent the micronized formulation of the respective co-crystal; and filled circles represent the nanosized formulation of the respective co-crystal.

### DETAILED DESCRIPTION OF THE INVENTION

“Co-crystal” as used herein means a crystalline material composed of two or more different molecules that co-exist in the crystalline unit cell with a defined stoichiometry and interact non-ionically and non-covalently. Herein, the co-crystals comprise at least one active pharmaceutical ingredient (API) and at least one co-crystal former (“co-former”). In certain embodiments, the “co-crystal” herein is a crystalline material composed of one active pharmaceutical ingredient (API) and one co-crystal former (“co-former”). “Non-ionic” as used herein refers to energetically favorable molecular interactions that are not considered an ionic bond, and includes, for example, hydrogen bonding.

In general, the “co-former” is not a solvent. Examples of solvents that are not co-formers include, but are not limited to, water, methanol, ethanol, isopropanol, propanol, butanol, dimethyl sulfoxide, ethyl acetate, isopropyl acetate, acetone, butanone, dimethylformamide, dimethylacetamide, N-methylpyrrolidinone, tetrahydrofuran, chloroform, and dichloromethane, propylene glycol, ethylene glycol, dimethyl carbonate, diethyl carbonate, ethylene carbonate, toluene, and xylene(s).

Examples of suitable co-formers include, but are not limited to, adipic acid, maleic acid, malonic acid, glycolic acid, gentisic acid, 4-hydroxybenzoic acid, (+)-camphoric acid, L-malic acid, aspirin (acetylsalicylic acid), 1-hydroxy-2-naphthoic acid, salicylic acid, glutaric acid, fumaric acid, succinic acid, adipic acid, benzoic acid, DL-malic acid, hydrocinnamic acid, ethyl maltol, and maltol. In certain embodiments, the co-former may be selected from, for example, the group consisting of 1-hydroxy-2-naphthoic acid, acetylsalicylic acid (aspirin), benzoic acid, maleic acid, 2,5-dihydroxybenzoic acid, 4-hydroxybenzoic acid, hydrocinnamic acid, succinic acid, and salicylic acid. In one embodiment, the co-former is 1-hydroxy-2-naphthoic acid (xinafoic acid), succinic acid, acetylsalicylic acid (aspirin), maleic acid, or salicylic acid. In one embodiment, the co-former is acetylsalicylic acid (aspirin) or salicylic acid. In another embodiment, the co-former is 1-hydroxy-2-naphthoic acid (xinafoic acid). In another embodiment, the co-former is acetylsalicylic acid (aspirin). In another embodiment, the co-former is salicylic acid. In another embodiment, the co-former is maleic acid. In another embodiment, the co-former is succinic acid.

Examples of suitable meloxicam co-crystals include:

TABLE 1

| | | Stoichiometry |
|---|---|---|
| API | Co-former | (API: Co-former) |
| meloxicam | (+) camphoric acid | 3:2 |
| meloxicam | 1-hydroxy-2-naphthoic acid | 1:1 |
| | (xinafoic acid) | |
| meloxicam | 2,5-dihydroxybenzoic acid | 1:1 |
| meloxicam | 4-hydroxybenzoic acid | 1:1 |
| meloxicam | adipic acid | 2:1 |
| meloxicam | aspirin (acetylsalicylic acid) | 1:1 |
| meloxicam | benzoic acid | 1:1 |
| meloxicam | DL-malic acid | 2:1 |
| meloxicam | ethyl maltol | 1:1 |
| meloxicam | fumaric acid | 2:1 |
| meloxicam | glutaric acid | 1:1 |
| meloxicam | glycolic acid | 1:1 |
| meloxicam | hydrocinnamic acid | 1:1 |
| meloxicam | L-malic acid | 2:1 |
| meloxicam | maleic acid | 1:1 |
| meloxicam | malonic acid | 1:1 |
| meloxicam | maltol | 1:1 |
| meloxicam | salicylic acid | 1:1 |
| meloxicam | succinic acid | 2:1 |

Meloxicam co-crystals may be prepared, for example, by dry or solvent grinding processes disclosed in U.S. Pat. No. 8,124,603. Alternatively, the co-crystals may be prepared by co-crystallizing from or solvent evaporation of a solution in a single solvent or a solvent mixture system; see, for example Indian Provisional Patent Application 201841041849, entitled “MELOXICAM CO-CRYSTALS”, filed on Nov. 5, 2018, and International Patent Application PCT/IN2019/050815, entitled “MELOXICAM CO-CRYSTALS”, filed on Nov. 4, 2019.

In the compositions herein, the meloxicam co-crystals may have a D90 ranging from about 100 nm up to 25000 nm (25 μm). In certain embodiments, the meloxicam co-crystals may be “micro-cocrystals” or “nano-crystals” as described below. Particle size distributions of an API may be determined, for example, by analytical sieving or light diffraction according to United States Pharmacopeia and National Formulary (USP 42-NF 37, May 1, 2019) Chapter (786) (Particle Size Distribution Estimation by Analytical Sieving and Chapter (429) (Light Diffraction Measurement of Particle Size), respectively, and may be classified in the following manner: D90 is the particle diameter corresponding to 90% of the cumulative undersize distribution; D50 is the median particle diameter (i.e., 50% of the particles are smaller and 50% of the particles are larger); and D10 is the particle diameter corresponding to 10% of the cumulative undersize distribution.

A “nano-cocrystal”, as used herein, means a co-crystal, as defined herein, having D90 of less than 5000 nm. In certain embodiments, the nano-cocrystal has D90 between about 100 nm and 5000 nm; or between about 100 nm and about 2000 nm; or between about 500 nm and 5000 nm; or between about 500 nm and about 2000 nm.

A “micro-cocrystal”, as used herein, means a co-crystal, as defined herein, having D90 of greater 5000 nm (5 μm), such as a D90 between 5000 nm (5 μm) and about 25000 nm (25 μm); or between 5000 nm (5 μm) and about 20000 nm (20 μm); or between 5000 nm (5 μm) and about 18000 nm (18 μm).

In one embodiment, a meloxicam micro-cocrystal is a characterized by a D10 between about 500 nm and 5000 nm (5 μm). In another embodiment, a meloxicam micro-cocrystal is characterized by a D50 between about 1000 nm and 10000 nm (10 μm). In another embodiment the meloxicam micro-cocrystal is characterized by a D90 between about 5000 nm (5 μm) and 20000 nm (20 μm). For example, the meloxicam micro-cocrystal can be characterized by a particle size distribution that is a D10 between 500 nm and 5000 nm (5 μm); a D50 between 1000 nm and 10000 nm (10 μm); and a D90 between 5000 nm (5 μm) and 20000 nm (20 μm).

In another embodiment, a meloxicam nano-cocrystal is a characterized by a D10 between about 25 nm and 500 nm; or between about 50 nm and 250 nm; or between about 50 nm and 200 nm. In another embodiment, a meloxicam nano-cocrystal is characterized by a D50 between about 100 nm and 1000 nm (1 μm); or between about 100 nm and 750 nm; or between about 100 nm and 500 nm. In another embodiment the meloxicam nano-cocrystal is characterized by a D90 between about 100 nm and 5000 nm (5 μm); or between about 250 nm and 2000 nm (2 μm); or between about 400 nm and 2000 nm (2 μm). For example, the meloxicam nano-cocrystal can be characterized by a particle size distribution that is a D10 between 50 nm and 250 nm; a D50 between 100 nm and 1000 nm (1 μm); and a D90 between 250 nm and 5000 nm (5 μm). Or, the meloxicam nano-cocrystal can be characterized by a particle size distribution that is a D10 between 50 nm and 200 nm; a D50 between 100 nm and 500 nm; and a D90 between 250 nm and 2000 nm (2 μm).

“About” as used herein means +/−10% of the referenced value. In certain embodiments, “about” means +/−9%, or +/−8%, or +/−7%, or +/−6%, or +/−5%, or +/−4%, or +/−3%, or +/−2+/− or +/−1% of the referenced value.

Different methods may be utilized to reduce the particle size of hydrophobic or poorly water-soluble drugs such as meloxicam. The micro-cocrystals and nano-cocrystals herein may be prepared through either chemical precipitation (bottom-up technology) or disintegration (top-down technology). The micro-cocrystals and nano-cocrystals can be obtained, for example, by processes such as but not limited to, sieving, milling (e.g., jet milling, wet milling, ball milling, or dry milling), precipitation, and homogenization.

For example, meloxicam nano-cocrystals may be obtained by milling (e.g., wet milling, dry milling, or jet milling) of meloxicam co-crystals having an D90 greater than 5000 nm to provide a meloxicam nano-cocrystal having a D90 between about 100 nm and 5000 nm.

In certain embodiments, the meloxicam nano-cocrystals may be obtained by wet milling of meloxicam co-crystals having an D90 greater than 5000 nm in the presence of a fluid carrier, such as water.

In another embodiment, the meloxicam nano-cocrystals may be prepared by dry or wet milling meloxicam and a co-former in a suitable stoichiometric ratio, for example, as shown in Table 1 above. In this embodiment, the solvent for wet milling can be, for example, chloroform, dichloromethane, tetrahydrofuran, ethyl acetate, butyl acetate, isopropyl acetate, acetone, 2-butanone, dioxane, methanol, ethanol, isopropanol, butanol, or a mixture thereof.

### Pharmaceutical Compositions

Meloxicam co-crystals of can be formulated into several solid dosage forms suitable for oral administration such as capsules, tablets, pills, powders, and granules; such solid dosages do not include oral solutions or oral suspensions. In one embodiment, an oral solid pharmaceutical composition can be prepared that comprises a meloxicam co-crystal (e.g., a meloxicam nano-cocrystal or micro-cocrystal) and one or more pharmaceutically acceptable excipients.

Suitable excipients may include, but are not limited to, one or more: (a) carriers, diluents, or fillers, (b) binders (e.g., polymeric binders), (c) humectants, (d) disintegrating agents, (e) solution retarders, (f) absorption accelerators, (g) wetting agents, (h) adsorbents, (i) lubricants, (j) surface stabilizers, (k) suspension stabilizers, (l) glidants, and (m) buffering agents. The pharmaceutical composition may comprise the meloxicam co-crystal in an amount of 1-50% by weight, more particularly 1-25% by weight; or 1-15% by weight, or 1-10% by weight, or 5-25% by weight, or 5-15% by weight, or 5-10% by weight, or 8-12% by weight of the composition. The pharmaceutical composition may also comprise the one or more pharmaceutically acceptable excipients in an amount of sufficient to bring the total to 100% of the composition (q.s. 100%).

Suitable pharmaceutically acceptable solid carriers, diluents, and fillers may include one or more saccharides, disaccharides, and sugar alcohols such as lactose (for example, spray-dried lactose, α-lactose, and β-lactose, such as lactose available under the trade marks TABLETTOSE and PHARMATOSE, available from MEGGLE Group Wasserburg, BG Excipients & Technology, Wasserburg, Germany), lactitol, sucrose, sorbitol, mannitol, dextrose; polysaccharide and polysaccharide derivatives such as dextrates, dextrin, maltodextrin, dextran, croscarmellose sodium, microcrystalline cellulose (for example, microcrystalline cellulose available under the trade mark AVICEL, FMC Corp., Philadelphia, Pa.), hydroxypropylcellulose, low-substituted hydroxypropylcellulose, hydroxypropyl methylcellulose (HPMC), methylcellulose polymers (such as, for example, METHOCEL A, METHOCEL A4C, METHOCEL A15C, METHOCEL A4M, Dow Chemical, Midland, Mich.), hydroxyethylcellulose, sodium carboxymethylcellulose, carboxymethyl hydroxyethylcellulose, starches (including potato starch, corn starch, maize starch and rice starch), pregelatinized starches, and modified starches; and mixtures thereof. A solid carrier may be used in an amount of 10-90% by weight, more particularly 25-75% by weight of the composition. A diluent may be used in an amount of 10-50% by weight, more particularly 10-40% by weight or 10-30% by weight, or 20-40% by weight, 20-30% by weight of the composition.

In one embodiment, each solid carrier is independently selected from the group consisting of a saccharide, a disaccharide, a sugar alcohol, a polysaccharide, and a polysaccharide derivative. In one embodiment, the solid carrier comprises a saccharide, a disaccharide, or a sugar alcohol; and a polysaccharide or polysaccharide derivative. In another embodiment, the solid carrier comprises a polysaccharide. In another embodiment, the solid carrier comprises a disaccharide and a polysaccharide. In another embodiment, the solid carrier comprises lactose, lactitol, sucrose, sorbitol, mannitol, dextrin, dextrose, maltodextrin, microcrystalline cellulose, starch, pregelatinized starch, or a mixture thereof. In another embodiment, the solid carrier comprises a saccharide, a disaccharide, or a sugar alcohol; and microcrystalline cellulose, pregelatinized starch, or a mixture thereof. In another embodiment, the solid carrier comprises a disaccharide and microcrystalline cellulose. In another embodiment, the solid carrier comprises a disaccharide, microcrystalline cellulose, and pregelatinized starch. In another embodiment, the solid carrier comprises lactose and microcrystalline cellulose. In another embodiment, the solid carrier comprises lactose, microcrystalline cellulose, and pregelatinized starch.

In another embodiment, when present, each diluent is independently selected from the group consisting of a saccharide, a disaccharide, a sugar alcohol, a polysaccharide, and a polysaccharide derivative. In one embodiment, wherein the diluent comprises one or more polysaccharide or polysaccharide derivative. In another embodiment, the diluent comprises maltodextrin, microcrystalline cellulose, starch, pregelatinized starch, or a mixture thereof. In another embodiment, the diluent comprises microcrystalline cellulose. In another embodiment, the diluent comprises microcrystalline cellulose and pregelatinized starch.

Binders may comprise (e.g., a polymeric binder), for example, polyvinylpyrrolidone (povidone); polyethylene glycol(s), polyethylene oxide, cellulose derivatives including ethyl cellulose, methyl cellulose, hydroxypropylcellulose, hydroxymethylcellulose, hydroxypropyl methylcellulose (HPMC), hydroxyethylcellulose, sodium carboxymethylcellulose, carboxymethyl hydroxyethylcellulose; modified starch derivatives such as hydroxypropyl starch or pregelatinized hydroxypropyl starch; polysaccharides including, starch and starch-based polymers e.g. pre-gelatinized starch; chitosan, alginates; maltodextrin; polysaccharide gums such as, but not limited to, acacia, locust bean gum, agar, dextrin, carrageenan, calcium carrageenan, casein, zein, alginic acid, sodium alginate, pectin, gelatin, xanthan gum, guar gum, fenugreek gum, gum arabic, galactomannans, gellan, konjac, inulin, karaya gum, gum tragacanth, and combinations thereof; and Carbomer homopolymers of acrylic acid, or Carbomer polymers of acrylic acid crosslinked with an allyl ether of pentaerythritol, sucrose, or propylene glycol. Binders may also include inorganic binders such as bentonite or magnesium aluminum silicate. The binder may be used in an amount of 0.1-10% by weight, more particularly 0.1-10% by weight; or 0.1-5% by weight; or 0.1-3% by weight; or 1-10% by weight; or 1-5% by weight; 1-3% by weight of the composition.

In one embodiment, wherein the binder comprises a hydrophilic polymer. In another embodiment, the binder may comprise one or more hydrophilic polymers selected from the group consisting of polyvinylpyrrolidone (povidone); polyethylene glycol, hydroxypropylcellulose, hydroxymethylcellulose, hydroxypropyl methylcellulose (HPMC), hydroxyethylcellulose, sodium carboxymethylcellulose, carboxymethyl hydroxyethylcellulose; hydroxypropyl starch or pregelatinized hydroxypropyl starch; pregelatinized starch; Carbomer homopolymers, and Crosslinked Carbomer polymers. In another embodiment, the binder may comprise one or more hydrophilic polymers selected from the group consisting of polyvinylpyrrolidone (povidone); polyethylene glycol, hydroxypropylcellulose, hydroxypropyl methylcellulose, hydroxypropyl starch or pregelatinized hydroxypropyl starch; and pregelatinized starch. In another embodiment, the binder may comprise one or more hydrophilic polymers selected from the group consisting of polyvinylpyrrolidone (povidone); and hydroxypropyl methylcellulose. In another embodiment, the polymeric binder comprises polyvinylpyrrolidone. In another embodiment, the polymeric binder comprises hydroxypropyl methylcellulose.

HPMC can be characterized by either its average molecular weight or the viscosity of a 2 wt. % solution in water at 20° C. “Molecular weight” as used herein refers to the weight-averaged molecular weight (M_{w}) of the referenced polymer. In certain embodiments, the binder is a low-viscosity HPMC, having a viscosity of a 2 wt. % solution in water at 20° C. of less about than 100 cP. In one example, the binder is a low-viscosity HPMC having a viscosity as measured in a 2 wt. % solution in water of about 2 cP to about 60 cP. In certain other embodiments, the binder is an HPMC having a viscosity as measured in a 2 wt. % solution in water of about 2 cP to about 10 cP. Examples of commercially available HPMCs that can be used as a binder herein include, but are not limited to, METHOCEL E3 LV (28-30% methoxy substitution, 7-12% hydroxypropyl substitution, 2.4-3.6 cP), METHOCEL E5 LV (28-30% methoxy substitution, 7-12% hydroxypropyl substitution, 4-6 cP), METHOCEL E6 LV (28-30% methoxy substitution, 7-12% hydroxypropyl substitution, 4.8-7.2 cP), METHOCEL E15 LV (28-30% methoxy substitution, 7-12% hydroxypropyl substitution, 12-18 cP), METHOCEL E50 LV (28-30% methoxy substitution, 7-12% hydroxypropyl substitution, 40-60 cP); METHOCEL is available from Dow Chemical Co., Midland, Mich.

Suitable disintegrating agents (or “disintegrants”) include, for example, hydroxypropyl cellulose (HPC), low substituted HPC (having a hydroxypropyl content of less than 15%, such as 8% or 11%), carboxymethylcellulose (CMC), sodium CMC, calcium CMC, crystalline cellulose, croscarmellose sodium, carboxymethyl starch, hydroxypropyl starch, alginic acid or a salt thereof such as sodium alginate, corn starch, potato starch, maize starch, modified starches, microcrystalline cellulose, crospovidone (e.g., POLYPLASDONE, POLYPLASDONE XL 10, both from Ashland, Covington, Ky.), sodium starch glycolate, and mixtures thereof. The disintegrant may be used in an amount of 0.1-25% by weight, more particularly 0.1-15% by weight; or 0.1-10% by weight; or 1-15% by weight; or 1-10% by weight, or 3-7% by weight of the composition.

In one embodiment, wherein the disintegrant comprises low substituted hydroxypropyl cellulose, carboxymethylcellulose sodium, croscarmellose sodium, crospovidone, and sodium starch glycolate. In another embodiment, the disintegrant comprises crospovidone.

“Surface stabilizer” as used herein, refers to surfactants that, without being limited by any one mode of operation, are believed to be capable of stabilizing the increased surface charge of a nanomilled drug, and may include amphoteric, non-ionic, cationic, or anionic surfactants.

Examples of amphoteric surfactants include, but are not limited to, lecithin, cocamidopropyl betaine, lauryldimethylamine oxide, myristamine oxide, coco amino propionate (SERVO AM 1010 Element's Specialties, Delden, The Netherlands), sodium lauryl imino dipropionate (SERVO AM 1020), sodium octyl imino dipropionate (SERVO AM 2020), sodium coco imino mono/dipropionate (SERVO AM 1015), oleyldimethylbetaine, and sodium N-cocoamidethyl N-hydroxyethylglycine, and mixtures thereof.

Examples of non-ionic surfactants include, but are not limited to, alkylphenols, such as 4-(2,4-dimethylheptan-3-yl)phenol; fatty acid glycerides such as glyceryl dibehenate, glyceryl monocaprylate, glyceryl monocaprylocaprate, glyceryl monostearate, and glyceryl tristearate; sorbitan esters such as sorbitan monolaurate, sorbitan monooleate, sorbitan monostearate, sorbitan sesqueoleate, sorbitan trioleate, and sorbitan tristearate; ethoxylated fatty acids such as stearic acid ethoxylate and lauric acid ethoxylate; ethoxylated fatty alcohols such as polyoxyethylene lauryl ethers (Brij); ethoxylated alkylphenols such as nonylphenol ethoxylate and ethoxylated p-tert-octylphenol; ethoxylated hydrogenated vegetable oils such as polyoxyl 35 castor oil (Cremophor EL) and hydrogenated polyoxyl 40 castor oil (Cremophor RH 40); ethoxylated sorbitan esters such as polyoxyethylene sorbitan monolaurate (polysorbate 20), polyoxyethylene sorbitan monopalmitate (polysorbate 40), polyoxyethylene sorbitan monostearate (polysorbate 60), and polyoxyethylene sorbitan monooleate (polysorbate 80); ethoxylated fatty acid amides such as cocoamide monoethanolamine and cocamide diethanolamine; and mixtures thereof.

Examples of cationic surfactants include, but are not limited to, quaternary ammonium salts such as cetyl trimethyl ammonium bromide (CTAB), methylbenzethonium chloride, and hexadecyltrimethylammonium bromide; 2-alkyl-1-hydroxyethyl-2-imidazolines such as lauryl hydroxyethyl imidazoline and stearyl hydroxyethyl imidazoline; N,N,N,N-tetrakis-substituted ethylenediamines such as ethylenediamine tetrakis(ethoxylate-block-propoxylate) tetrol and ethylenediamine tetrakis(propoxylate-block-ethoxylate) tetrol; fatty amine ethoxylates such as stearyl amine ethoxylate, oleyl amine ethoxylate, tallow amine ethoxylate and coco amine ethoxylate; and mixtures thereof.

Examples of anionic surfactants include, but are not limited to, alkyl sulfates, such as sodium dodecyl sulfate (sodium lauryl sulfate) and ammonium lauryl sulfate; bile salts such as sodium deoxycholate and sodium cholate; sulfosuccinate diesters such as docusate sodium, ammonium dinonyl sulfosuccinate, diamyl sulfosuccinate sodium, dicapryl sulfosuccinate sodium, diheptyl sulfosuccinate sodium, dihexyl sulfosuccinate sodium, diisobutyl sulfosuccinate sodium, and ditridecyl sulfosuccinate sodium; alkylbenzene sulfonates such as sodium dodecylbenzenesulfonate; sodium petroleum sulfonates such as those available under the tradename PETRONATE from Sonneborn, LLC Petrolia, Pa.; sodium alkyl naphthalene sulfonate such as those available under the tradename NAXAN from Nease Performance Chemicals, West Chester Township, Ohio; sulfated natural oils and glycerides such as lauryl monoglyceryl sulfate and sulfated castor oil; and sulfated ethoxylated fatty alcohols such as sodium laureth sulfate and sodium myreth sulfate; and mixtures thereof.

The surface stabilizer may be used in an amount of less than 5% by weight of the composition, for example, 0.1-5% by weight; and more particularly 0.1-2% by weight; or 0.1-1% by weight, of the composition. In one embodiment, the surface stabilizer comprises sodium lauryl sulfate, ammonium lauryl sulfate, docusate sodium, ammonium dinonyl sulfosuccinate, diamyl sulfosuccinate sodium, dicapryl sulfosuccinate sodium, diheptyl sulfosuccinate sodium, dihexyl sulfosuccinate sodium, diisobutyl sulfosuccinate sodium, ditridecyl sulfosuccinate sodium, sodium dodecylbenzenesulfonate, or a mixture thereof. In another embodiment, the surface stabilizer comprises sodium lauryl sulfate.

“Suspension stabilizer” as used herein refers to excipients that may prevent physical interaction and/or flocculation of solid particles therein. Examples of suitable suspension stabilizers include, but are not limited to, sugars, sugar alcohols, and sugar derivatives, such as lactose, sucrose, hydrolyzed starch (maltodextrin) and mixtures thereof. The suspension stabilizer may be used in an amount of 1-25% by weight, more particularly 1-15% by weight; or 1-10% by weight; or 5-25% by weight; or 5-15% by weight; or 5-10% by weight of the composition. In one embodiment, the suspension stabilizer comprises sorbitol or sucrose. In one embodiment, the suspension stabilizer comprises sorbitol. In one embodiment, the suspension stabilizer comprises sucrose.

Glidants may comprise one or more, but not limited to talc; powdered cellulose; calcium phosphate (e.g., tribasic calcium phosphate); magnesium oxide; sodium stearate; silicic acid or a derivative or salt thereof (for example, silicates, calcium silicate, magnesium silicate, magnesium trisilicate, silicon dioxide, colloidal silicon dioxide, hydrophobic silicon dioxide, and polymers thereof); magnesium aluminosilicate (such as NEUSILIN, available from Fuji Chem. Indus. Co. Ltd., Japan); magnesium alumina metasilicate; and mixtures thereof. The glidants may be used in an amount of 0.1-5% by weight, more particularly 0.1-3% by weight; or 0.1-2% by weight; or 0.1-1% by weight of the composition.

In one embodiment, wherein the glidant comprises talc, calcium phosphate, calcium silicate, magnesium silicate, magnesium trisilicate, silicon dioxide, colloidal silicon dioxide, magnesium aluminosilicate, or a mixture thereof. In another embodiment, wherein the glidant comprises talc, calcium phosphate, silicon dioxide, colloidal silicon dioxide, or a mixture thereof. In another embodiment, the glidant comprises silicon dioxide.

Lubricants may comprise one or more, but not limited to fatty acids such as lauric acid, myristic acid, palmitic acid, and stearic acid and pharmaceutically acceptable salts or esters thereof (for example, magnesium stearate, calcium stearate, sodium stearyl fumarate, zinc stearate or other metallic stearate); talc; polyethylene glycols (PEGs); light mineral oil; poloxamers, such as KOLLIPHOR P188 and P407 available from BASF, Ludwigshafen, Germany; polysorbates such as polysorbate 20, polysorbate 40, polysorbate 60, and polysorbate 80; sodium lauryl sulfate; sorbitan esters such as sorbitan monolaurate, sorbitan monooleate, sorbitan monopalmitate, sorbitan monostearate, sorbitan sesquioleate and sorbitan trioleate; ethoxylated fatty acids such as polyoxyl 40 stearate and polyoxyl 15 hydroxystearate; ethoxylated alkanols such as polyoxyl 20 cetostearyl ether and polyoxyl 10 oleyl ether; ethoxylated vegetable oils and ethoxylated hydrogenated vegetable oils such as polyoxyl 35 castor oil (Cremophor EL) and hydrogenated polyoxyl 40 castor oil (Cremophor RH 40); waxes (for example, microcrystalline waxes); glycerides, such as glyceryl dibehenate, glyceryl monocaprylate, glyceryl monocaprylocaprate, glyceryl monostearate, and glyceryl tristearate; sucrose ester of fatty acids such as sucrose stearate; hydrogenated vegetable oils (for example, hydrogenated castor oil and hydrogenated palm oil); and mixtures thereof.

In one embodiment, the lubricant comprises lauric acid, myristic acid, palmitic acid, stearic acid or pharmaceutically acceptable salts or esters thereof, such as magnesium stearate, calcium stearate, sodium stearyl fumarate, or zinc stearate or a mixture thereof. In another embodiment the lubricant comprises stearic acid, magnesium stearate, calcium stearate, sodium stearyl fumarate, zinc stearate, or a mixture thereof. In another embodiment, the lubricant comprises magnesium stearate, calcium stearate, sodium stearyl fumarate, zinc stearate or a mixture thereof. In one embodiment, the lubricant comprises stearic acid, magnesium stearate, or a mixture thereof. In one embodiment, the lubricant comprises magnesium stearate. In another embodiment, the lubricant comprises stearic acid. The lubricant may be used in an amount of 0.1-5% by weight, more particularly 0.1-3% by weight; or 0.1-2% by weight; or 0.1-1%; or 0.5-5% by weight, more particularly 0.5-3% by weight; or 0.5-2% by weight of the composition.

Buffering agents may include one or more of, but not limited to, acetic acid, tartaric acid, maleic acid, fumaric acid, glycine, lactic acid, lysine, maleic acid, malic acid, glutamic acid, citric acid, succinic acid, and salts thereof, such as sodium citrate dihydrate. In one embodiment, wherein the buffering agent comprises sodium succinate, sodium citrate, sodium glutamate, sodium acetate, sodium lactate, or a mixture thereof. In another embodiment, wherein the buffering agent comprises sodium succinate, sodium citrate, sodium lactate, or a mixture thereof. In one embodiment, the buffering agent comprises trisodium citrate (e.g., trisodium citrate dihydrate). The buffering agent may each be used in an amount of 0.1-10% by weight, more particularly 0.1-5% by weight; or 1-10% by weight; or 1-5% by weight, of the composition.

In one embodiment, the pharmaceutical composition comprises a meloxicam co-crystal (e.g., micro- or nano-cocrystal) and one or more pharmaceutically acceptable excipients that are independently selected from the group consisting of a polymeric binder, a surface stabilizer, a suspension stabilizer, a lubricant, a disintegrant, a glidant, a solid carrier, a buffering agent, and a mixture thereof.

In another embodiment, the pharmaceutical composition comprises a meloxicam nano-cocrystal and one or more pharmaceutically acceptable excipients that are a polymeric binder, a solid carrier, a surface stabilizer, a suspension stabilizer, a lubricant, a disintegrant, a glidant, and a buffering agent.

In one particular example of compositions comprising a meloxicam nano-cocrystal, the pharmaceutical composition may comprise a granulate and one or more pharmaceutically acceptable extragranular excipients, wherein the granulate comprises a meloxicam nano-cocrystal having a D90 between about 100 nm and 5000 nm and one or more pharmaceutically acceptable intragranular excipients. In one embodiment of compositions comprising a meloxicam nano-cocrystal, the one or more intragranular excipients are independently selected from the group consisting of a solid carrier, polymeric binder, a surface stabilizer, a suspension stabilizer, and mixtures thereof. In another embodiment, the one or more intragranular excipients are a solid carrier, a polymeric binder, a surface stabilizer, and a suspension stabilizer.

In another embodiment of compositions comprising a meloxicam nano-cocrystal, the one or more intragranular excipients comprise a solid carrier comprises a saccharide, a disaccharide, or a sugar alcohol; and a polysaccharide or polysaccharide derivative. a polymeric binder selected from the group consisting of polyvinylpyrrolidone, polyethylene glycol, hydroxypropylcellulose, hydroxymethylcellulose, hydroxypropyl methylcellulose, hydroxyethylcellulose, sodium carboxymethylcellulose, carboxymethyl hydroxyethylcellulose, hydroxypropyl starch, pregelatinized hydroxypropyl starch, pregelatinized starch, Carbomer homopolymers, crosslinked Carbomer polymers, and mixtures thereof; a surface stabilizer selected from the group consisting of sodium lauryl sulfate, ammonium lauryl sulfate, docusate sodium, ammonium dinonyl sulfosuccinate, diamyl sulfosuccinate sodium, dicapryl sulfosuccinate sodium, diheptyl sulfosuccinate sodium, dihexyl sulfosuccinate sodium, diisobutyl sulfosuccinate sodium, ditridecyl sulfosuccinate sodium, sodium dodecylbenzenesulfonate, or a mixture thereof; and a suspension stabilizer selected from the group consisting of lactose, sucrose, sorbitol, maltodextrin, and mixtures thereof.

In another embodiment of compositions comprising a meloxicam nano-cocrystal, the one or more intragranular excipients comprise a solid carrier comprises a saccharide, a disaccharide, or a sugar alcohol; and microcrystalline cellulose; a polymeric binder selected from the group consisting of polyvinyl pyrrolidone, polyethylene glycol, hydroxypropylcellulose, hydroxypropyl methylcellulose, hydroxypropyl starch, pregelatinized hydroxypropyl starch, pregelatinized starch, and mixtures thereof; a surface stabilizer comprises, a disaccharide and microcrystalline cellulose; and a suspension stabilizer selected from the group consisting of lactose, sucrose, sorbitol, and mixtures thereof.

In another embodiment of compositions comprising a meloxicam nano-cocrystal, the one or more intragranular excipients comprise lactose, microcrystalline cellulose, hydroxypropyl methylcellulose, sodium lauryl sulfate, and sucrose.

In one embodiment of compositions comprising a meloxicam nano-cocrystal, the extragranular excipients are independently selected from the group consisting of a lubricant, a disintegrant, a glidant, a buffering agent; and mixtures thereof. In certain embodiments, the extragranular excipients comprise a lubricant, a disintegrant, a glidant, and a buffering agent.

In one embodiment of compositions comprising a meloxicam nano-cocrystal, the extragranular excipients comprise:

a lubricant selected from the group consisting of lauric acid, myristic acid, palmitic acid, stearic acid or pharmaceutically acceptable salts or esters thereof, such as magnesium stearate, calcium stearate, sodium stearyl fumarate, zinc stearate and mixtures thereof;

a disintegrant selected from the group consisting of low substituted hydroxypropyl cellulose, carboxymethylcellulose sodium, croscarmellose sodium, crospovidone, and sodium starch glycolate and mixtures thereof;

a glidant selected from the group consisting of talc, calcium phosphate, calcium silicate, magnesium silicate, magnesium trisilicate, silicon dioxide, colloidal silicon dioxide, magnesium aluminosilicate, and mixtures thereof;

and a buffering agent selected from the group consisting of sodium succinate, sodium citrate, sodium glutamate, sodium acetate, sodium lactate, and mixtures thereof.

In another embodiment of compositions comprising a meloxicam nano-cocrystal, the extragranular excipients comprise:

a lubricant selected from the group consisting of stearic acid, magnesium stearate, calcium stearate, sodium stearyl fumarate, zinc stearate and mixtures thereof;

a disintegrant selected from the group consisting of low substituted hydroxypropyl cellulose, carboxymethylcellulose sodium, croscarmellose sodium, crospovidone, sodium starch glycolate, and mixtures thereof;

a glidant selected from the group consisting of talc, calcium phosphate, silicon dioxide, colloidal silicon dioxide, and mixtures thereof;

and a buffering agent selected from the group consisting of sodium succinate, sodium citrate, sodium lactate, and mixtures thereof.

In other embodiments of compositions comprising a meloxicam nano-cocrystal, the extragranular excipients comprise a magnesium stearate or stearic acid; crospovidone, silicon dioxide, and trisodium citrate

In another embodiment of compositions comprising a meloxicam nano-cocrystal, the one or more intragranular excipients comprise lactose, microcrystalline cellulose, hydroxypropyl methylcellulose, sodium lauryl sulfate, and sucrose; and the extragranular excipients comprise a magnesium stearate or stearic acid; crospovidone, silicon dioxide, and trisodium citrate.

In another embodiment of compositions comprising a meloxicam nano-cocrystal, the composition comprises: 5-15 wt. % meloxicam nano-cocrystal; 1-5 wt. % polymeric binder; 5-10 wt. % suspension stabilizer; and 65-80 wt. % solid carrier.

In another embodiment of compositions comprising a meloxicam nano-cocrystal, the composition comprises: 5-15 wt. % meloxicam nano-cocrystal; 1-5 wt. % polymeric binder; 0.1-1 wt. % surfactant; 5-10 wt. % suspension stabilizer; 65-80 wt. % solid carrier; 1-5 wt. % buffering agent; 1-10 wt. % disintegrant; 0.1-3 wt. % glidant; and 0.5-3 wt. % lubricant.

The pharmaceutical compositions may also comprise a meloxicam micro-cocrystal and one or more pharmaceutically acceptable excipients that are a polymeric binder, a solid carrier, a disintegrant, a lubricant, a glidant, a diluent, and a buffering agent.

In one particular example of compositions comprising a meloxicam micro-cocrystal, the pharmaceutical composition may comprise a granulate and one or more pharmaceutically acceptable extragranular excipients, wherein the granulate comprises a meloxicam micro-cocrystal and one or more pharmaceutically acceptable intragranular excipients.

In one embodiment of compositions comprising a meloxicam micro-cocrystal, the one or more intragranular excipients are independently selected from the group consisting of a solid carrier, polymeric binder, a disintegrant, and mixtures thereof. In another embodiment of compositions comprising a meloxicam micro-cocrystal, the one or more intragranular excipients are a solid carrier, polymeric binder, a disintegrant. In another embodiment, the one or more intragranular excipients comprise

a solid carrier comprising a saccharide, a disaccharide, or a sugar alcohol; and at least one polysaccharide or polysaccharide derivative;

a polymeric binder selected from the group consisting of polyvinylpyrrolidone, polyethylene glycol, hydroxypropylcellulose, hydroxymethylcellulose, hydroxypropyl methylcellulose, hydroxyethylcellulose, sodium carboxymethylcellulose, carboxymethyl hydroxyethylcellulose, hydroxypropyl starch, pregelatinized hydroxypropyl starch, pregelatinized starch, Carbomer homopolymers, crosslinked Carbomer polymers, and mixtures thereof; and

a disintegrant selected from low substituted hydroxypropyl cellulose, carboxymethylcellulose sodium, croscarmellose sodium, crospovidone, and sodium starch glycolate;

In another embodiment of compositions comprising a meloxicam micro-cocrystal, the one or more intragranular excipients comprise a solid carrier comprising a saccharide, a disaccharide, or a sugar alcohol; and pregelatinized starch and/or microcrystalline cellulose;

a polymeric binder selected from the group consisting of polyvinylpyrrolidone, polyethylene glycol, hydroxypropylcellulose, hydroxypropyl methylcellulose, and mixtures thereof;

and a disintegrant selected from carboxymethylcellulose sodium, croscarmellose sodium, crospovidone, and sodium starch glycolate;

In another embodiment of compositions comprising a meloxicam micro-cocrystal, the one or more intragranular excipients comprise lactose, polyvinylpyrrolidone, crospovidone, and pregelatinized starch or microcrystalline cellulose.

In one embodiment of compositions comprising a meloxicam micro-cocrystal, the extragranular excipients are independently selected from the group consisting of a diluent, a lubricant, a disintegrant, a glidant, a buffering agent; and mixtures thereof. In certain embodiments, the extragranular excipients comprise a diluent, a lubricant, a glidant, a buffering agent, and an optional disintegrant.

In one embodiment of compositions comprising a meloxicam micro-cocrystal, the extragranular excipients comprise:

a diluent selected from the group consisting of maltodextrin, croscarmellose sodium, microcrystalline cellulose, hydroxypropylcellulose, hydroxypropyl methylcellulose, methylcellulose, hydroxyethylcellulose, sodium carboxymethylcellulose, carboxymethyl hydroxyethylcellulose, potato starch, corn starch, maize starch, rice starch, and pregelatinized starch, and mixtures thereof;

a lubricant selected from the group consisting of lauric acid, myristic acid, palmitic acid, stearic acid and pharmaceutically acceptable salts or esters thereof, such as magnesium stearate, calcium stearate, sodium stearyl fumarate, or zinc stearate, and mixtures thereof;

a glidant selected from the group consisting of talc, calcium phosphate, calcium silicate, magnesium silicate, magnesium trisilicate, silicon dioxide, colloidal silicon dioxide, magnesium aluminosilicate, and mixtures thereof;

a buffering agent selected from the group consisting of sodium succinate, sodium citrate, sodium glutamate, sodium acetate, sodium lactate, and mixtures thereof; and

an optional disintegrant selected from the group consisting of low substituted hydroxypropyl cellulose, carboxymethylcellulose sodium, croscarmellose sodium, crospovidone, and sodium starch glycolate and mixtures thereof.

In another embodiment of compositions comprising a meloxicam micro-cocrystal, the extragranular excipients comprise:

a diluent selected from the group consisting of microcrystalline cellulose, hydroxypropylcellulose, hydroxypropyl methylcellulose, methylcellulose, hydroxyethylcellulose, carboxymethyl hydroxyethylcellulose, and pregelatinized starch, and mixtures thereof;

a lubricant selected from the group consisting of stearic acid, magnesium stearate, calcium stearate, sodium stearyl fumarate, zinc stearate, and mixtures thereof;

a glidant selected from the group consisting of talc, calcium phosphate, silicon dioxide, colloidal silicon dioxide, and mixtures thereof;

a buffering agent selected from the group consisting of sodium succinate, sodium citrate, sodium lactate, and mixtures thereof; and

an optional disintegrant selected from the group consisting of low substituted hydroxypropyl cellulose, carboxymethylcellulose sodium, croscarmellose sodium, crospovidone, sodium starch glycolate, and mixtures thereof.

In other embodiments of compositions comprising a meloxicam micro-cocrystal, the extragranular excipients comprise a magnesium stearate or stearic acid; silicon dioxide, trisodium citrate, and optionally, crospovidone.

In another embodiment of compositions comprising a meloxicam micro-cocrystal, the one or more intragranular excipients comprise lactose, polyvinylpyrrolidone, crospovidone, and pregelatinized starch or microcrystalline cellulose; and the extragranular excipients comprise a magnesium stearate or stearic acid; silicon dioxide, trisodium citrate, and optionally, crospovidone.

The meloxicam oral solid pharmaceutical compositions are also stable. For example, stability studies conducted on the 15 mg meloxicam nanosized tablet formulation (3) of meloxicam: salicylic acid (1:1) co-crystals described in Example 4 (“MECC-SA”) showed no significant meloxicam degradation (less than 2% w/w) over 6 months under both long term stability conditions (25° C. (±2° C.) and 60% (±5%) relative humidity) and accelerated stability conditions (40° C. (±2° C.) and 75% (±5%) relative humidity).

Process

Oral solid pharmaceutical compositions may be prepared according to a process comprising, preparing a granulate comprising a meloxicam co-crystal (e.g., micro- or nano-cocrystal) and one or more intragranular excipients; and combining the granulate with one or more extragranular excipients to provide a blend. Embodiments of intragranular and extragranular excipients are as described above.

For example, a granulate may be prepared by combining a meloxicam co-crystal (e.g., micro- or nano-cocrystal) with one or more intragranular excipients to provide a mixture and dry or wet granulating the mixture in the presence of a binder.

In one embodiment, the granulate can be prepared by wet granulation. Suitable wet granulation can include preparing a mixture of the meloxicam co-crystal, one or more intraparticulate excipients, and optionally a binder; and granulating the mixture with a solution comprising the binder. In certain embodiments, both the mixture and solution comprise a portion of the binder. In certain other embodiments, both only the solution comprises the binder. Such granulation may use a solution comprising the binder and water or a non-aqueous solvent, such as an alcohol (e.g., methanol, ethanol, isopropanol, and mixtures thereof). Non-aqueous solvent should be selected such that the co-crystal is essentially insoluble in the solvent. Such granulated particles can be dried and milled and/or sieved to provide a granulate having the desired handling and physical properties for additional processing into the final composition.

In one particular example, a pharmaceutical composition may be prepared using a granulate in process which comprises: preparing a mixture of a meloxicam co-crystal, a disintegrant, and one or more solid carriers; preparing a solution of a polymeric binder in a solvent; wet granulating the mixture and solution (e.g., by spraying the solution onto the mixture in a fluidized bed granulator) to provide a granulate; optionally drying the granulate; blending the optionally dried granulate with one or more extragranular excipients (e.g., a diluent, a glidant, a buffering agent, and a lubricant) to provide a blend; and compressing the blend into tablets.

Where the composition comprises a meloxicam nano-cocrystal, a granulate may be prepared, for example, by milling (e.g., wet milling, dry milling, or jet milling) a meloxicam co-crystal having D90 greater than 5000 nm, to provide a meloxicam nano-cocrystal having D90 between about 100 nm and 5000 nm. The nano-cocrystal may be combined and blended with one or more intragranular excipients and the mixture subsequently granulated by wet or dry methods familiar to those skilled in the art to provide the granulate.

In another example, the granulate may be prepared by forming a suspension of the meloxicam nano-cocrystal in a fluid carrier; and granulating one or more intragranular excipients with the suspension. Suitable fluid carriers include such solvents in which the meloxicam co-crystal is poorly soluble, including, but not limited to, water.

In one example, the fluid carrier may be combined with a previously prepared nano-cocrystal (e.g., by dry milling) to prepare the suspension comprising the meloxicam nano-cocrystal. In another example, a meloxicam co-crystal having D90 greater than 5000 nm, may be wet milled in the presence of the fluid carrier to provide the suspension comprising the meloxicam nano-cocrystal.

In certain embodiments, one or more intragranular excipients may be dissolved in the fluid carrier prior to or after the wet milling. For example, the fluid carrier may be a solution comprising water and one or more intragranular excipients selected from the group consisting of a polymeric binder, a surface stabilizer, a suspension stabilizer, and mixtures thereof. In certain embodiments the fluid carrier may be a solution comprising at least one surface stabilizer. In another embodiment, the fluid carrier may be a solution comprising at least one polymeric binder. In another embodiment the fluid carrier may be a solution comprising at least one suspension stabilizer. In certain embodiments the fluid carrier may be a solution comprising at least one surface stabilizer and at least one polymeric binder. In certain embodiments the fluid carrier may be a solution comprising at least one surface stabilizer and at least suspension stabilizer. In certain embodiments the fluid carrier may be a solution comprising at least one polymeric binder and at least one suspension stabilizer. In another example, the fluid carrier may be a solution comprising water, a polymeric binder, a surface stabilizer, and a suspension stabilizer. In another example, the fluid carrier may be a solution comprising water, sodium lauryl sulfate, hydroxypropyl methylcellulose, and sucrose. When the fluid carrier comprises water, the pH of the fluid carrier or the solution comprising the fluid carrier may be adjusted to have a pH between about 2.0 and 5.0; or between 2.0 and 4.0; or between 2.0 and 3.0.

Granulating the suspension prepared above with one or more additional intragranular excipients yields the granulates described above. Suitable intragranular excipients for the granulating process include one or more solid carriers as described in any of the preceding embodiments. In certain embodiments, the solid carriers comprise lactose and/or microcrystalline cellulose. Accordingly, in one embodiment, the resulting granules comprise the meloxicam nano-cocrystal, a polymeric binder, surface stabilizer, suspension stabilizer, and solid carrier. The resulting granules may be optionally dried and/or sieved to assist with subsequent handling and/or processes as needed.

A portion of any such blends as described above may be filled into a capsule shell or compressed to provide a solid dosage (e.g., tablets or caplets). The portion of the blend filled into a capsule or compressed can contain a desired amount of the meloxicam co-crystal. For example, the portion can contain the meloxicam co-crystal in an amount equivalent to 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10 mg, 10.5 mg, 11.0 mg, 11.5 mg, 12.0 mg, 12.5 mg, 13.0 mg, 13.5 mg, 14.0 mg, 14.5 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 32 mg, 34 mg, 36 mg, 38 mg, 40 mg, 42 mg, 44 mg, 46 mg, 48 mg, 50 mg, 52 mg, 54 mg, 56 mg, 58 mg, or 60 mg meloxicam base. “Meloxicam base” as used herein refers to meloxicam alone. In certain embodiments, the amount of meloxicam co-crystal is an amount equivalent to about 1-60 mg, or about 1-40 mg, or about 1-35 mg, or about 1-30 mg, or about 1-25 mg, or about 1-20 mg, or about 1-15 mg, or about 1-10 mg, or about 5-60 mg, or about 5-40 mg, or about 5-35 mg, or about 5-30 mg, or about 5-25 mg, or about 5-20 mg, or about 5-15 mg, or about 5-10 mg meloxicam base. In other embodiments, the amount of meloxicam co-crystal is an amount equivalent to 5.0 mg, 7.5 mg, 10 mg, 12.5 mg, 15 mg, 20 mg, 25 mg, or 30 mg meloxicam base.

According to one embodiment, a pharmaceutical composition may be prepared by a process which comprises: preparing a suspension comprising meloxicam co-crystals, a polymeric binder, a surfactant, a suspension stabilizer, and water; nanomilling the suspension to provide a suspension of meloxicam nano-cocrystals; wet granulating the suspension of meloxicam nano-cocrystals with one or more solid carriers to provide granules (e.g., by spraying the suspension of meloxicam nano-cocrystals onto the one or more solid carriers in a fluidized bed granulator); optionally drying the granules; blending the optionally dried granules with extragranular excipients (e.g., a buffering agent, a disintegrant, a glidant, and a lubricant) to provide a blend; and compressing the blend into tablets.

According to another embodiment, a pharmaceutical composition may be prepared by a process which comprises: preparing a suspension comprising meloxicam co-crystals, hydroxypropyl methylcellulose, sodium lauryl sulphate, sucrose, and water; nanomilling the suspension to provide a suspension of meloxicam nano-cocrystals; wet granulating the suspension of meloxicam nano-cocrystals with lactose and microcrystalline cellulose to provide granules (e.g., by spraying the suspension of meloxicam nano-cocrystals onto a lactose and microcrystalline cellulose mixture in a fluidized bed granulator); optionally drying the granules; blending the optionally dried granules with extragranular excipients to provide a blend; and compressing the blend into tablets.

Generally, any of the preceding dosage forms may be optionally coated (film-coated or non-film-coated). The film formers used for the coating process may, for example, be cellulose derivatives such as methyl cellulose (MC), ethyl cellulose (EC), hydroxyethyl cellulose (HEC), methacrylic acid/acrylate copolymers, HPMC, vinyl polymers or natural film formers, such as shellac. Examples of commercially available film formers include, but are not limited to, Opadry® (HPMC), Opadry® II (poly(vinyl alcohol)), and Surelease® (Ethylcellulose Dispersion Type B NF) Film Coating Systems (each available from Colorcon, Inc., North Wales, Pa.), and mixtures thereof.

### Dissolution and Pharmacokinetics

Surprisingly, the rate of release of meloxicam in the co-crystal compositions (e.g., micro- and/or nano-cocrystal) provided herein were both substantially greater in several aqueous dissolution media than MOBIC tablets or VIVLODEX capsules, showing greater potential for the rapid exposure (e.g., as measured by blood plasma concentration (ng/mL) or partial area under the curve as a function of time, pAUC(0-t)) that can enable improved treatment of pain (and in particular, acute pain). This faster release can lead to a substantially faster rate of uptake of meloxicam in vivo as demonstrated below, in particular as compared to MOBIC tablets.

Rate of API release of any of the compositions provided herein can be measured in a USP Apparatus 2 (Paddle Apparatus) at 37+/−2° C., according to the methods of USP42-NF37 chapter (711) on dissolution, which is incorporated herein by reference. For example, the rate of meloxicam release can be measured in a USP Apparatus 2 (Paddle Apparatus) at 37+/−2° C., in 900 mL of dissolution media selected from 0.1 N HCl, acetate buffer (pH 4.5), and phosphate buffer (pH 6.1).

“pAUC(0-t)” as used herein refer to the partial area under the curve (AUC) that is the definite integral in the plot of mean drug concentration in blood plasma versus time for the time between 0 hours and t hours after dose administration, for a cohort of patients. For example, “pAUC(0-2)” and “pAUC(0-4)” refer to the partial area under the curve (AUC) for the time between 0 hours and 2 hours or 4 hours after dose administration, respectively.

In one embodiment, an oral solid composition is provided comprising a meloxicam co-crystal (e.g., an amount equivalent to about 1 mg to about 60 mg, or 5 mg, 10 mg, 15 mg, 20 mg, 25 mg, or 30 mg meloxicam base), and one or pharmaceutically acceptable excipients and the meloxicam is released in 900 mL of 0.1N HCl as measured by a USP-II Apparatus at 75 rpm and 37±2° C. according to one of: greater than about 30 wt. %, about 40 wt. %, about 50 wt. %, about 60 wt. %, or about 70 wt. % at 60 minutes; or greater than about 30 wt. %, about 40 wt. %, about 50 wt. %, or about 60 wt. % at 30 minutes; or greater than about 30 wt. %, about 40 wt. %, or about 50 wt. % at 15 minutes.

In another embodiment, oral solid compositions are provided comprising a meloxicam co-crystal (e.g., an amount equivalent to about 1 mg to about 60 mg, or 5 mg, 10 mg, 15 mg, 20 mg, 25 mg, or 30 mg meloxicam base), and a pharmaceutically acceptable excipient and the meloxicam is released in an acetate buffer as measured in a USP-II Apparatus at 75 rpm and 37±2° C. according to one of: greater than about 30 wt. %, about 40 wt. %, about 50 wt. %, about 60 wt. %, or about 70 wt. %, or about 80 wt. % at 60 minutes; greater than about 30 wt. %, about 40 wt. %, about 50 wt. %, or about 60 wt. %, or about 70 wt. % at 30 minutes; or greater than about 30 wt. %, about 40 wt. %, or about 50 wt. %, or about 60 wt. % at 15 minutes.

In another embodiment, oral solid compositions are provided comprising a meloxicam co-crystal (e.g., an amount equivalent to about 1 mg to about 60 mg, or 5 mg, 10 mg, 15 mg, 20 mg, 25 mg, or 30 mg meloxicam base), and a pharmaceutically acceptable excipient and the meloxicam is released in a phosphate buffer (pH 6.1) as measured in a USP-II Apparatus at 75 rpm and 37±2° C. according to one of: greater than about 90 wt. % or about 95 wt. % at 15 minutes; greater than about 80 wt. %, about 85 wt. %, or about 90 wt. % at 10 minutes; or greater than about 70 wt. %, about 75 wt. %, about 80 wt. %, about 85 wt. %, or about 90 wt. % at 5 minutes.

In certain embodiments, the meloxicam co-crystal of the co-former (“Co-”) noted below is released according to one of the following embodiments (1)-(24):

| | | (0.1N HCl, 900 mL, | | | (Acetate buffer, pH 4.5, | | | (Phosphate buffer, pH 6.1, | | |
|---|---|---|---|---|---|---|---|---|---|---|
| | | USP-II) | | | 900 mL, USP-II) | | | 900 mL, USP-II) | | |
| | | Meloxicam release (wt. %) | | | | | | | | |
| # | Co- | 15 min | 30 min | 60 min | 15 min | 30 min | 60 min | 5 min | 15 min | 30 min |
| 1 | a | 30-65 | — | — | — | — | — | — | — | — |
| 2 | a | — | 30-65 | — | — | — | — | — | — | — |
| 3 | a | — | — | 30-65 | — | — | — | — | — | — |
| 4 | a | — | — | — | 50-75 | — | — | — | — | — |
| 5 | a | — | — | — | — | 55-85 | — | — | — | — |
| 6 | a | — | — | — | — | — | 60-90 | — | — | — |
| 7 | a | — | — | — | — | — | — | 75-85 | — | — |
| 8 | a | — | — | — | — | — | — | — | 85-95 | — |
| 9 | a | — | — | — | — | — | — | — | — | >90 |
| 10 | b | — | — | — | 20-40 | — | — | — | — | — |
| 11 | b | — | — | — | — | 25-45 | — | — | — | — |
| 12 | b | — | — | — | — | — | 30-50 | — | — | — |
| 13 | b | — | — | — | — | — | — | 70-85 | — | — |
| 14 | b | — | — | — | — | — | — | — | >85 | — |
| 15 | b | — | — | — | — | — | — | — | — | >90 |
| 16 | c | 45-70 | — | — | — | — | — | — | — | — |
| 17 | c | — | 50-80 | — | — | — | — | — | — | — |
| 18 | c | — | — | 50-80 | — | — | — | — | — | — |
| 19 | c | — | — | — | 55-90 | — | — | — | — | — |
| 20 | c | — | — | — | — | 60-90 | — | — | — | — |
| 21 | c | — | — | — | — | — | 65-95 | — | — | — |
| 22 | c | — | — | — | — | — | — | >80 | — | — |
| 23 | c | — | — | — | — | — | — | — | >90 | — |
| 24 | c | — | — | — | — | — | — | — | — | >95 |

(a) Succinic acid;
(b) Xinafoic acid;
(c) Salicylic acid

Treatment of Pain

In another aspect, the pharmaceutical compositions herein are useful in methods for the prevention or treatment of pain. As used here, the terms “treatment” and “treating” means (i) ameliorating the referenced disease state, for example, ameliorating a disease, condition or disorder in an individual who is experiencing or displaying the pathology or symptomatology of the disease, condition or disorder (i.e., reversing or improving the pathology and/or symptomatology) such as decreasing the severity of disease; or (ii) eliciting the referenced biological effect (e.g., reduction in the perception of pain). As used here, the terms “prevention” and “preventing” means preventing a disease, condition or disorder in an individual who may be predisposed to the disease, condition or disorder but does not yet experience or display the pathology or symptomatology of the disease; for example, by administration of a compositions as described herein prior to or in anticipation of a surgical event, a strenuous activity or other event.

Examples of pain which may be treated with a pharmaceutical composition herein include, but are not limited to, peripheral, central or muscle skeletal pain; and/or acute, subacute or chronic pain; and/or moderate to severe pain; and/or neuropathic or psychogenic or nociceptive or mixed pain; and/or low back pain, visceral pain or headache; and/or post-operative (post-surgical), cancer or inflammatory pain.

As used herein, “acute pain” refers to pain that may have a known cause, such as due to an injury or surgery, and lasts less than about 4 weeks; for example, less than about 2 weeks, or about 10-14 days. “Subacute pain” refers to pain that lasts from more than about 4 weeks to about 12 weeks, and “chronic pain” refers to pain that lasts for more than about 12 weeks.

Examples of acute pains include, but are not limited to pain caused by post-operative pain including pain following spinal fusion, laminectomy/discectomy, hip replacement, knee arthroplasty, hip fracture repair, mastectomy, coronary artery bypass graft, hernia repair, small-bowel resection/enterolysis, bunionectomy, cholecystectomy, hysterectomy, appendectomy, colectomy, thyroidectomy, Cesarean section, cholecystectomy, appendectomy, and tooth extraction (e.g., molar tooth extraction).

The chronic pain may be selected from the group consisting of cancer pain, peripheral neuropathic pain, osteoarthritis, rheumatoid arthritis, juvenile rheumatoid arthritis, chronic visceral pain, neuropathic pain (diabetic polyneuropathy, HIV-associated neuropathic pain, posttraumatic neuropathic pain, postherpetic neuralgia, chemotherapy associated pain), postzosteric neuralgia, inflammatory pain, migraine, lower-back pain, fibromyalgia, and trigeminal neuralgia.

In one embodiment, the pain is chronic pain, such as chronic nociceptive and/or chronic inflammatory pain.

In another embodiment, the pain is subacute or acute pain, such as post-operative (post-surgical) pain. In another embodiment, the pain is selected from acute or subacute postoperative neuropathic pain and inflammatory pain.

Patients in need of treatment for any of the preceding pain conditions may be treated by administering to the person in need of such treatment an oral solid composition as described and prepared above. A particular subset of patients that may be treated include persons diagnosed with an opioid use disorder. “Opioid use disorder” (OUD) can be diagnosed either using the Diagnostic and Statistical Manual of Mental Disorders, Fifth Edition (DSM-5) assessment criteria, or as previously classified as Opioid Abuse or Opioid Dependence in DSM-IV. See, for example, www.cdc.gov/drugoverdose/training/oud/accessible/index.html.

Administration of the oral solid compositions herein may be once-daily or more, such as twice-daily administration, where the same dosage of the meloxicam co-crystal is provided at each administration. “Once daily administration” (sid) means that the pharmaceutical dosage form herein is adapted for being consecutively administered according to a regimen comprising the administration of a pharmaceutical dose once per day, wherein the time interval between the consecutive administration of two pharmaceutical doses is at least 18 hours, or at least 20 hours, or at least 22 hours, or about 24 hours. For example, the dosage form may be packaged with instructions that instruct a patient in need thereof to take a suitable pharmaceutical dose once per day, or every 24 hours.

“Twice daily administration” (bid) means that the pharmaceutical dosage form herein is adapted for being consecutively administered according to a regimen comprising the administration of two pharmaceutical doses per day, wherein the time interval between the consecutive administration of two pharmaceutical doses is at least 9 hours, or at least 10 hours, or at least 11 hours, or about 12 hours. For example, the dosage form may be packaged with instructions that instruct a patient in need of thereof to take a suitable pharmaceutical dose twice per day, or every about 9 to 12 hours, or every 12 hours. In another example, the dosage form may be packaged with instructions that instruct a patient in need of thereof to take a suitable pharmaceutical dose twice per day, or every about 9 to 12 hours, or every 12 hours, and on an empty stomach at each dosing. In one embodiment, a daily therapeutically effective amount (i.e., total amount of drug administered within a single day) of meloxicam co-crystal of an amount equivalent to between about 10 mg and 60 mg meloxicam base can be divided (e.g., equally divided) between the two administrations; such as, 10 mg or 15 mg bid, for a daily therapeutically effective amount of 20 mg and 30 mg, respectively.

Advantageously, the meloxicam co-crystal compositions herein are capable of providing rapid absorption of meloxicam, particularly when administered to a fasting patient as is known to those skilled in the art (e.g., on an “empty stomach” as defined herein). In any preceding instance, the dosage form may be packaged with instructions that instruct a patient in need thereof to take the suitable pharmaceutical dose on an empty stomach, and particularly, for the treatment of acute pain. Instructions for taking the dosages “on an empty stomach”, include, for example, instructions to take the dosages at least 2 hours, or at least 3 hours, or at least 4 hours after the patient's previous meal. In another instance, the dosage form may be packaged with instructions that instruct a patient in need thereof to take the suitable pharmaceutical dose on a full stomach, and particularly, for the treatment of chronic pain. Instructions for taking the dosages “on a full stomach”, include, for example, instructions to take the dosages with a meal or within 2 hours or less after the patient's previous meal.

Actual dosage levels of meloxicam (i.e., meloxicam co-crystals) in the compositions may be varied to obtain an amount of meloxicam co-crystal that is effective to obtain a desired biological or medicinal response for a particular composition, method of administration, and particular disease, condition or disorder for treatment or prevention (i.e., “therapeutically effective amount”). The selected dosage level therefore depends upon the desired therapeutic effect, the route of administration, the potency of the administered meloxicam, the desired duration of treatment, and other factors, such as age and gender of the subject.

Advantageously, the meloxicam co-crystal compositions herein are capable of providing rapid absorption of meloxicam, resulting in higher mean blood plasma concentrations of meloxicam in a reduced period of time, particularly when administered to a person on an empty stomach, as defined herein. Such mean blood plasma concentrations can be measured in a single-dose pharmacokinetic study. A “single-dose pharmacokinetic study” can be performed as is familiar to those skilled in the art, for example, according to Examples 7 or 8, herein, utilizing a cohort of adult fasting human subjects, such as 15-20 subjects.

For example, the oral solid compositions can comprise a meloxicam co-crystal and a pharmaceutically acceptable excipient that provides a therapeutic blood concentration of meloxicam in less than about 3 hours as measured in a post-surgical dental pain study. A “post-surgical dental pain study” can be performed as is familiar to those skilled in the art, for example, according to the trial described in Example 11 in a cohort 100-120 adult fasting subjects.

Similarly, the meloxicam co-crystal compositions herein are capable of providing a reduction of rescue medication use by patients in need of treatment for acute pain, for example, for patients following surgery, such as molar extraction surgery. “Rescue medications”, as used herein, includes those familiar to those skilled in the art, including opioid containing medications that useful for the treatment of breakthrough pain as known to one skilled in the art. Rescue medications include, for example, immediate release opioid containing medications, such as hydrocodone/acetaminophen (5/500 mg or 5/325 mg). For example, the compositions here can provide for at a reduction in rescue medication use as compared to patients receiving placebo as measured in a post-surgical dental pain study. In another example, the compositions here can provide for at a reduction in rescue medication use as compared to patients receiving placebo tablets as measured in a post-surgical dental pain study. In each of the preceding embodiments, the reduction of rescue medication use is at least 5%, 10%, 15%, 20%, 25%, 30%, 35%, 40%, 45%, 50%, 55%, 60%, 65%, 70%, 75%, 80%, 85%, 90%, 95%, or 100%.

In certain embodiments, the compositions herein can be considered opioid-sparing medications. “Opioid sparing” as used herein mean decreasing the amount of opioid taken for analgesia while achieving comparable analgesic benefit. Opioid sparing can be demonstrated through a reduction of mean mg opioid used; a difference in mean mg opioid used expressed as % of use in relation to placebo group; and/or an increase in the % subjects that used no opioid, each as compared to placebo and/or standard of care.

In certain embodiments, the compositions herein can provide improved duration of analgesia with respect to MOBIC tablets. “Duration of analgesia” as used herein means the median time to a request for rescue or re-medication, as determined in a cohort of patients in a relevant clinical study. For example, the compositions herein can provide at least 5%, 10%, 15%, 20%, 25%, 30%, 35%, 40%, 45%, 50%, 55%, 60%, 65%, 70%, 75%, 80%, 85%, 90%, 95%, or 100% improvement in duration of analgesia as compared to placebo tablets in a post-surgical dental pain study.

The term “therapeutic blood concentration” as used herein refers to the mean blood plasma concentration of meloxicam necessary for a desired clinical effect. That is, the desired clinical effect (such as analgesia) can be observed when the blood plasma concentration for a subject achieves at least the “therapeutic blood concentration.” For example, for the treatment of pain (including acute pain), the therapeutic blood concentration for meloxicam can be about 500 ng/mL or greater, such as 550 ng/mL, or about 600 ng/mL, or about 650 ng/mL, or about 700 ng/mL, or about 750 ng/mL, or about 800 ng/mL, or about 850 ng/mL, or about 900 ng/mL, or about 950 ng/mL, or about 1000 ng/mL, or about 1050 ng/mL, or about 1090 ng/mL, or about 1100 ng/mL, or about 1150 ng/mL, or about 1200 ng/mL, or about 1250 ng/mL or about 1300 ng/mL, or about 1350 ng/mL, or about 1400 ng/mL, or about 1450 ng/mL, or about 1500 ng/mL, or about 1550 ng/mL, or about 1600 ng/mL, or about 1650 ng/mL, or about 1700 ng/mL, or about 1750 ng/mL, or about 1800 ng/mL, or about 1850 ng/mL, or about 1900 ng/mL, or about 1950 ng/mL, or about 2000 ng/mL, or a bioequivalent thereof.

“Bioequivalent” as used herein means, with respect to a particular PK value (e.g., Cₘₐₓ, AUC, pAUC), that the 90% confidence interval of the geometric least squares mean ratio of the natural log-transformed parameter value falls between 80-125% of the recited (compared) value.

In certain embodiments, the oral solid compositions herein can provide at least the therapeutic blood concentration of meloxicam in less than about 4 hours, or less than about 3 hours, or less than about 2.5 hours, or less than about 2 hours, or less than about 1.5 hours, or less than about 1.0 hour, or less than about 0.5 hour. For example, the compositions herein can provide at least the therapeutic blood concentration of meloxicam between about 0.25 hour and 4 hours, or about 0.25 hour and 3.5 hours, about 0.25 hour and 3 hours, or between about 0.25 hour and 2.5 hours, or between about 0.25 hour and 2.0 hours, or between about 0.25 hour and 1.5 hours, or between about 0.25 hour and 1.0 hours, or between about 0.25 hour and 0.75 hour, or between about 0.25 hour and 0.50 hour, or about 0.5 hour and 4 hours, or about 0.5 hour and 3.5 hours, or about 0.5 hour and 3 hours, or between about 0.5 hour and 2.5 hours, or between about 0.5 hour and 2.0 hours, or between about 0.5 hours and 1.5 hours, or between about 0.5 hour and 1.0 hour, or between about 0.5 hour and 0.75 hour, or about 0.75 hour and 4 hours, or about 0.75 hour and 3.5 hours, or about 0.75 hour and 3 hours, or between about 0.75 hour and 2.5 hours, or between about 0.75 hour and 2.0 hours, or between about 0.75 hour and 1.5 hours, or between about 0.75 hour and 1.0 hour, between about 1.0 hour and 4.0 hours, about 1.0 hour and 3.5 hours, about 1.0 hour and 3.0 hours, or between about 1.0 hour and about 2.5 hours, or between about 1.0 hour and about 2.0 hours, or between about 1.0 hour and about 1.5 hours, or between about 1.5 hours and 4.0 hours, or between about 1.5 hours and 3.5 hours, or between about 1.5 hours and 3.0 hours, or between about 1.5 hours and 2.5 hours, or between about 1.5 hours and 2.0 hours, or between about 2.0 hours and 4.0 hours, or between about 2.0 hours and 3.5 hours, or between about 2.0 hours and 3.0 hours, or between about 2.0 hours and 2.5 hours.

For example, a single dose of a 10-15 mg (meloxicam base) oral solid compositions of a meloxicam co-crystal can provide a blood plasma concentration of between 500-2500 ng/mL, or a bioequivalent thereof, (e.g., 1000-2500 ng/mL, or a bioequivalent thereof) in less than about 2 hours (or at about 2 hours). In another example, a single dose of a 10-15 mg (meloxicam base) dosage of a meloxicam co-crystal can provide a blood plasma concentration of between 1000-2500 ng/mL, or a bioequivalent thereof, in less than about 2 hours (or at about 2 hours) and a blood plasma concentration of between 500-1500 ng/mL, or a bioequivalent thereof, in less than about 0.5 hour (or at about 0.5 hour).

A single dose of a 15 mg (meloxicam base) oral solid composition of a meloxicam co-crystal can provide a blood plasma concentration of between 500-2500 ng/mL, or a bioequivalent thereof, (e.g., 1000-2500 ng/mL, or a bioequivalent thereof) in less than about 2 hours. In another example, a 15 mg (meloxicam base) dosage of a meloxicam co-crystal can provide a blood plasma concentration of between 1000-2500 ng/mL, or a bioequivalent thereof, in less than about 2 hours (or at about 2 hours) and a blood plasma concentration of between 500-1500 ng/mL, or a bioequivalent thereof, in less than about 0.5 hour (or at about 0.5 hour).

A single dose of a 10 mg (meloxicam base) oral solid composition of a meloxicam co-crystal can provide a blood plasma concentration of between 500-1500 ng/mL, or a bioequivalent thereof, (e.g., 500-1000 ng/mL, 1000-1500 ng/mL, or a bioequivalent thereof) in less than about 2 hours (or at about 2 hours). In another example, a 10 mg (meloxicam base) dosage of a meloxicam co-crystal can provide a blood plasma concentration of between 1000-1500 ng/mL, or a bioequivalent thereof, in less than about 2 hours (or at about 2 hours) and a blood plasma concentration of between 500-1000 ng/mL, or a bioequivalent thereof, in less than about 0.5 hour (or at about 0.5 hour).

Where the therapeutic blood concentration is about 1000 ng/mL (e.g., between 800 ng/mL and 1250 ng/mL), a single dose of a 10-15 mg (meloxicam base) oral solid composition of a meloxicam co-crystal can provide at least the therapeutic blood concentration between about 0.25 hour and 2 hours, or between about 0.25 hour and 1.75 hours, or between about 0.25 hour and 1.5 hours, or between about 0.25 hour and 1.25 hours, or between about 0.25 hour and 1.0 hours, or between about 0.5 hour and 2 hours, or between about 0.5 hour and 1.75 hours, or between about 0.5 hour and 1.5 hours, or between about 0.5 hour and 1.25 hour, or between about 0.5 hour and 1 hour, or between 0.75 hour and 2 hours, or between about 0.75 hour and 1.75 hours, or between about 0.75 hour and 1.5 hours, or between about 0.75 hour and 1.25 hours, or between about 0.75 hour and 1.0 hours, or between 1.0 hour and 2 hours, or between about 1.0 hour and 1.75 hours, or between about 1.0 hour and 1.5 hours, or between about 1.0 hour and 1.25 hours. For example a 10 mg (meloxicam base) oral solid composition of a meloxicam co-crystal can provide at least the therapeutic blood concentration between about 0.75 hour and 2 hours (e.g., at about 0.75 h, or 1 hour, or 1.25 hour, or 1.5 hour, 1.75 hour, or 2 hours) and a 15 mg dosage provide at least the therapeutic blood concentration between about 0.25 hour and 2 hours (e.g., at about 0.25 h, or 0.35 h, or 0.45 h, or 0.50 h, or 0.75 h, or 1 hour).

Where the therapeutic blood concentration is about 1090 ng/mL (e.g., between 870 ng/mL and 1365 ng/mL), a single dose of a 10-15 mg (meloxicam base) oral solid composition of a meloxicam co-crystal can provide at least the therapeutic blood concentration between about 0.25 hour and 2 hours, or between about 0.25 hour and 1.75 hours, or between about 0.25 hour and 1.5 hours, or between about 0.25 hour and 1.25 hours, or between about 0.25 hour and 1.0 hours, or between about 0.5 hour and 2 hours, or between about 0.5 hour and 1.75 hours, or between about 0.5 hour and 1.5 hours, or between about 0.5 hour and 1.25 hour, or between about 0.5 hour and 1 hour, or between 0.75 hour and 2 hours, or between about 0.75 hour and 1.75 hours, or between about 0.75 hour and 1.5 hours, or between about 0.75 hour and 1.25 hours, or between about 0.75 hour and 1.0 hours, or between 1.0 hour and 2 hours, or between about 1.0 hour and 1.75 hours, or between about 1.0 hour and 1.5 hours, or between about 1.0 hour and 1.25 hours. For example a single dose of a 10 mg (meloxicam base) oral solid composition of a meloxicam co-crystal can provide at least the therapeutic blood concentration between about 0.75 hour and 2 hours (e.g., at about 0.75 h, or 1 hour, or 1.25 hour, or 1.5 hour, 1.75 hour, or 2 hours) and a 15 mg dosage provide at least the therapeutic blood concentration between about 0.25 hour and 1 hours (e.g., at about 0.25 h, or 0.35 h, or 0.45 h, or 0.50 h, or 0.75 h, or 1 hour).

Where the therapeutic blood concentration is about 1200 ng/mL (e.g., between 960 ng/mL and 1500 ng/mL), a single dose of a 10-15 mg (meloxicam base) oral solid composition of a meloxicam co-crystal can provide at least the therapeutic blood concentration between about 0.5 hour and 2 hours, or between about 0.5 hour and 1.5 hour. For example a single dose of a 10 mg (meloxicam base) dosage of a meloxicam co-crystal can provide at least the therapeutic blood concentration between about 1 hour and 2 hours (e.g., at about 1 hour, or 1.25 hour, or 1.5 hour, 1.75 hour, or 2 hours) and a single dose of a 15 mg dosage provide at least the therapeutic blood concentration between about 0.5 hour and 1 hours (e.g., at about 0.25 h, or 0.35 h, or 0.45 h, or 0.50 h, or 0.75 h, or 1 hour).

Where the therapeutic blood concentration is about 1500 ng/mL (e.g., between 1200 ng/mL and 1875 ng/mL), a single dose of a 15 mg (meloxicam base) oral solid composition of a meloxicam co-crystal can provide at least the therapeutic blood concentration between about 0.5 hour and 2 hours, or between about 0.5 hour and 1.5 hour. For example, the single dose of a 15 mg dosage provide at least the therapeutic blood concentration between about 0.5 hour and 1.25 hours (e.g., at about 0.50 h, or 0.60 h, or 0.70 h, or 0.75 h, or 0.80 h, or 0.90 h, or 1 hour).

The achievement of a therapeutic blood concentration can be manifested through perceptible pain relief and/or meaningful pain relief, which can be accessed via the double-stopwatch method. At dosing, two stopwatches are started; the time of first perceptible relief can be recorded as the time the first stopwatch is stopped and is defined as post-dose time at which a subject first begins to feel pain relief. Time of meaningful relief can be recorded as the time the second stopwatch is stopped and is defined as the post-dose time at which a subject begins to feel meaningful pain relief.

In certain embodiments, the composition described herein can provide a mean time of first perceptible relief of between about 5-180 minutes as measured in a post-surgical dental pain study. For example, mean time of first perceptible relief can be between about 5-165, or 5-150, or 5-135, or 5-120, or 5-105, or 5-90, or 5-75, or 5-60, or 5-45, or 5-30, or 10-180, or 10-165, or 10-150, or 10-135, or 10-120, or 10-105, or 10-90, or 10-75, or 10-60, or 10-45, or 10-30, or 15-180, or 15-165, or 15-150, or 15-135, or 15-120, or 15-105, or 15-90, or 15-75, or 15-60, or 15-45, or 15-30, or 20-180, or 20-165, or 20-150, or 20-135, or 20-120, or 20-105, or 20-90, or 20-75, or 20-60, or 20-45, or 20-30, or 30-180, or 30-165, or 30-150, or 30-135, or 30-120, or 30-105, or 30-90, or 30-75, or 30-60, or 30-45, or 40-180, or 40-165, or 40-150, or 40-135, or 40-120, or 40-105, or 40-90, or 40-75, or 40-60, or 50-180, or 50-165, or 50-150, or 50-135, or 50-120, or 50-105, or 50-90, or 50-75, or 60-180, or 60-165, or 60-150, or 60-135, or 60-120, or 60-105, or 60-90, or 75-180, or 75-165, or 75-150, or 75-135, or 75-120, or 75-105, or 90-180, or 90-165, or 90-150, or 90-135, or 90-120 minutes.

In certain embodiments, the composition described herein can provide a mean time of meaningful relief between about 15-240 minutes as measured in a post-surgical dental pain study. For example, mean time of meaningful relief can be between about 15-225, or 15-210, or 15-195, or 15-180, or 15-165, or 15-150, or 15-135, or 15-120, or 15-105, or 15-90, or 30-240, or 30-225, or 30-210, or 30-195, or 30-180, or 30-165, or 30-150, or 30-135, or 30-120, or 30-105, or 30-90, or 45-240, or 45-225, or 45-210, or 45-195, or 45-180, or 45-165, or 45-150, or 45-135, or 45-120, or 45-105, or 45-90, or 60-240, or 60-225, or 60-210, or 60-195, or 60-180, or 60-165, or 60-150, or 60-135, or 60-120, or 60-105, or 60-90, or 75-240, or 75-225, or 75-210, or 75-195, or 75-180, or 75-165, or 75-150, or 75-135, or 75-120, or 75-105, or 90-240, or 90-225, or 90-210, or 90-195, or 90-180, or 90-165, or 90-150, or 90-135, or 90-120, or 105-240, or 105-225, or 105-210, or 105-195, or 105-180, or 105-165, or 105-150, or 105-135, or 120-240, or 120-225, or 120-210, or 120-195, or 120-180, or 120-165, or 120-150 minutes.

In another embodiment, including any of the preceding release rate embodiments and/or any off the preceding therapeutic blood concentration embodiments, an oral solid composition is provided comprising a meloxicam co-crystal and a pharmaceutically acceptable excipient, wherein a therapeutically effective amount of a meloxicam co-crystal (e.g., equivalent to about 1 mg to about 60 mg, or 5 mg, 10 mg, or 15 mg meloxicam base), and having one or more of:

| | PK parameter | Range (between about) | | or A:MOBIC tablets |
|---|---|---|---|---|
| (a) | Cₘₐₓ | 1500-3000 | ng/mL | or 1.2-2.0 times; |
| (b) | pAUC(0-1) | 400-1200 | ng · h/mL | or 2.5-7.0 times; |
| (c) | pAUC(0-2) | 1200-3500 | ng · h/mL | or 2.0-5.5 times; |
| (d) | pAUC(0-3) | 2500-5500 | ng · h/mL | or 1.3-5.0 times; |
| (e) | pAUC(0-4) | 4000-7500 | ng · h/mL | or 1.2-3.2 times; |
| (f) | pAUC(0-6) | 7000-12000 | ng · h/mL | or 1.1-2.5 times; |
| (g) | median Tₘₐₓ | 1.5-3.0 | hours | |

or a bioequivalent thereof, wherein each of the preceding PK ranges are measured in a single-dose pharmacokinetic study or a post-surgery dental pain study, and where “A: MOBIC tablets” is the geometric least squares mean ratio of the referenced natural log-transformed PK parameter for the meloxicam co-crystal composition (A) to MOBIC tablets in the same cohort of subjects in a pharmacokinetic study (in fasting subjects). For example, in this embodiment, the meloxicam co-crystal can be selected from the group consisting of meloxicam: succinic acid (2:1); meloxicam: aspirin (1:1), meloxicam: xinafoic acid (1:1), meloxicam: salicylic acid (1:1), and meloxicam: maleic acid (1:1).

In another embodiment, the meloxicam co-crystal is meloxicam: succinic acid (2:1), and the composition is characterized by one or more of:

| | PK parameter | Range (between about) | | or A:MOBIC tablets |
|---|---|---|---|---|
| (a) | Cₘₐₓ | 1800-2500 | ng/mL | or 1.3-1.7 times; |
| (b) | pAUC(0-1) | 700-1100 | ng · h/mL | or 3.0-5.0 times; |
| (c) | pAUC(0-2) | 1800-2600 | ng · h/mL | or 2.0-3.5 times; |
| (d) | pAUC(0-3) | 3000-5000 | ng · h/mL | or 1.3-2.8 times; |
| (e) | pAUC(0-4) | 4000-7000 | ng · h/mL | or 1.2-2.3 times; |
| (f) | pAUC(0-6) | 7000-11000 | ng · h/mL | or 1.1-2.0 times; |
| (g) | median Tₘₐₓ | 1.5-3.0 | hours | |
| (h) | Cₘₐₓ | 2000-2400 | ng/mL | or 1.4-1.6 times; |
| (i) | pAUC(0-1) | 750-1000 | ng · h/mL | or 3.5-4.6 times; |
| (j) | pAUC(0-2) | 2000-2400 | ng · h/mL | or 2.0-3.0 times; |
| (k) | pAUC(0-3) | 3300-4300 | ng · h/mL | or 1.5-2.5 times; |
| (l) | pAUC(0-4) | 5200-6200 | ng · h/mL | or 1.4-2.0 times; |
| (m) | pAUC(0-6) | 8000-10000 | ng · h/mL | or 1.2-2.0 times; |
| (n) | median Tₘₐₓ | 2.0 and 3.0 | hours; | |

or a bioequivalent thereof, wherein each of the preceding PK ranges are measured in a single-dose pharmacokinetic study or a post-surgery dental pain study, and where “A: MOBIC tablets” is the geometric least squares mean ratio of the referenced natural log-transformed PK parameter for the meloxicam: succinic acid (2:1) composition (A) to MOBIC tablets in the same cohort of subjects in a pharmacokinetic study (in fasting subjects).

In another embodiment, the meloxicam co-crystal is meloxicam: xinafoic acid (1:1), and composition is characterized by one or more of:

| | PK parameter | Range (between about) | | or A:MOBIC tablets |
|---|---|---|---|---|
| (a) | Cₘₐₓ | 1600-2600 | ng/mL | or 1.3-2.0 times; |
| (b) | pAUC(0-1) | 400-1200 | ng · h/mL | or 2.5-6.0 times; |
| (c) | pAUC(0-2) | 1200-3000 | ng · h/mL | or 2.2-5.0 times; |
| (d) | pAUC(0-3) | 2500-5000 | ng · h/mL | or 2.0-4.5 times; |
| (e) | pAUC(0-4) | 4000-7000 | ng · h/mL | or 1.6-3.0 times; |
| (f) | pAUC(0-6) | 7000-11000 | ng · h/mL | or 1.2-2.2 times; |
| (g) | median Tₘₐₓ | 1.5-3.0 | hours; | |
| (h) | Cₘₐₓ | 2000-2400 | ng/mL | or 1.3-1.8 times; |
| (i) | pAUC(0-1) | 400-800 | ng · h/mL | or 3.0-4.5 times; |
| (j) | pAUC(0-2) | 1300-2000 | ng · h/mL | or 2.5-4.0 times; |
| (k) | pAUC(0-3) | 3000-4000 | ng · h/mL | or 2.0-4.0 times; |
| (l) | pAUC(0-4) | 4500-5500 | ng · h/mL | or 1.7-2.6 times; |
| (m) | pAUC(0-6) | 7500-10000 | ng · h/mL | or 1.3-2.0 times; |
| (n) | median Tₘₐₓ | 2.0 and 3.0 | hours; | |

or a bioequivalent thereof, wherein each of the preceding PK ranges are measured in a single-dose pharmacokinetic study or a post-surgery dental pain study, and where “A: MOBIC tablets” is the geometric least squares mean ratio of the referenced natural log-transformed PK parameter for the meloxicam: xinafoic acid (1:1) composition (A) to MOBIC tablets in the same cohort of subjects in a pharmacokinetic study (in fasting subjects).

In another embodiment, the meloxicam co-crystal is meloxicam: salicylic acid (1:1), and the composition is characterized by one or more of:

| | PK parameter | Range (between about) | | or A:MOBIC tablets |
|---|---|---|---|---|
| (a) | Cₘₐₓ | 1500-3000 | ng/mL | or 1.2-2.0 times; |
| (b) | pAUC(0-1) | 400-1500 | ng · h/mL | or 3.0-7.0 times; |
| (c) | pAUC(0-2) | 1200-3500 | ng · h/mL | or 2.5-5.5 times; |
| (d) | pAUC(0-3) | 2500-5500 | ng · h/mL | or 2.0-5.0 times; |
| (e) | pAUC(0-4) | 3500-7500 | ng · h/mL | or 1.6-3.2 times; |
| (f) | pAUC(0-6) | 6000-12000 | ng · h/mL | or 1.4-2.5 times; |
| (g) | median Tₘₐₓ | 1.5-3.0 | hours; | |
| (h) | Cₘₐₓ | 1600-2800 | ng/mL | or 1.3-1.8 times; |
| (i) | pAUC(0-1) | 400-1200 | ng · h/mL | or 3.0-6.5 times; |
| (j) | pAUC(0-2) | 1500-3000 | ng · h/mL | or 2.8-5.0 times; |
| (k) | pAUC(0-3) | 3000-5000 | ng · h/mL | or 2.0-4.0 times; |
| (l) | pAUC(0-4) | 4000-7000 | ng · h/mL | or 1.8-3.0 times; |
| (m) | pAUC(0-6) | 7000-11000 | ng · h/mL | or 1.5-2.2 times; |
| (n) | median Tₘₐₓ | 1.5-2.5 | hours; | |

or a bioequivalent thereof, wherein each of the preceding PK ranges are measured in a single-dose pharmacokinetic study or a post-surgery dental pain study, and where “A: MOBIC tablets” is the geometric least squares mean ratio of the referenced natural log-transformed PK parameter for the meloxicam: salicylic acid (1:1) composition (A) to MOBIC tablets in the same cohort of subjects in a pharmacokinetic study (in fasting subjects).

Pharmacokinetic values as expressed herein, unless otherwise noted, are mean values as calculated for a relevant cohort of patients via the referenced values of the individual patients. For example, pAUC(0-t), Cₘₐₓ, kₑᵢ, and t_{1/2} herein are expressed as mean values. Ratios of pharmacokinetic parameters are expressed the geometric least squares mean ratio of the natural log-transformed PK parameters. Such pharmacokinetic values and ratios may be derived from either a single-dose pharmacokinetic study or within another study by measuring the pharmacokinetic values associated with a first dose administered to each patient in the study cohort. For example, in a dental pain study, pharmacokinetic values may be calculated from the first dose of medication administered in the study; an example of a suitable dental pain study includes a post-surgical dental pain in a cohort of 100-120 adult fasting subjects according to the trial described in Example 11.

### Exemplary Embodiments

[Embodiment 1] A process for preparing a solid pharmaceutical composition comprising, preparing a granulate comprising a meloxicam co-crystal (e.g., a meloxicam micro-cocrystal or nano-cocrystal) and one or more intragranular excipients; and combining the granulate with one or more extragranular excipients to provide a blend.

[Embodiment 2] The process of Embodiment 1, wherein the granulate is prepared by forming a suspension of the meloxicam co-crystal (e.g., a meloxicam micro-cocrystal or nano-cocrystal) in a fluid carrier; and granulating one or more intragranular excipients with the suspension.

[Embodiment 3] The process of [Embodiment 2], wherein a suspension of a meloxicam nano-cocrystal is prepared by milling a meloxicam co-crystal having D90 greater than about 5000 nm.

[Embodiment 4] The process of [Embodiment 3], wherein the milling is dry milling, wet milling, or jet milling. [Embodiment 5] The process of [Embodiment 3], wherein the suspension of the meloxicam nano-cocrystal is prepared by wet milling the meloxicam co-crystal in the presence of a fluid carrier.

[Embodiment 6] The process of [Embodiment 5], wherein the fluid carrier comprises water. [Embodiment 7] The process of any one of [Embodiments 2-6], wherein the fluid carrier is a solution comprising water and one or more intragranular excipients selected from the group consisting of a polymeric binder, a surface stabilizer, a suspension stabilizer, and mixtures thereof.

[Embodiment 8] The process of [Embodiment 7], wherein the solution comprises water, a polymeric binder, a surface stabilizer, and a suspension stabilizer.

[Embodiment 9] The process of [Embodiment 7 or 8], wherein the polymeric binder comprises a hydrophilic polymer. [Embodiment 10] The process of [Embodiment 7 or 8], wherein the polymeric binder comprises polyvinylpyrrolidone, hydroxypropyl methylcellulose, and mixtures thereof.

[Embodiment 11] The process of [Embodiment 7 or 8], wherein the polymeric binder comprises hydroxypropyl methylcellulose.

[Embodiment 12] The process of any one of [Embodiments 7-11], wherein the surface stabilizer is a surfactant, a sugar, a sugar alcohol, or a sugar derivative (e.g., a surfactant).

[Embodiment 13] The process of any one of [Embodiments 7-11], wherein the surface stabilizer comprises sodium lauryl sulfate, ammonium lauryl sulfate, docusate sodium, ammonium dinonyl sulfosuccinate, diamyl sulfosuccinate sodium, dicapryl sulfosuccinate sodium, diheptyl sulfosuccinate sodium, dihexyl sulfosuccinate sodium, diisobutyl sulfosuccinate sodium, ditridecyl sulfosuccinate sodium, sodium dodecylbenzenesulfonate, or a mixture thereof.

[Embodiment 14] The process of any one of [Embodiments 7-11], wherein the surface stabilizer comprises sodium lauryl sulfate.

[Embodiment 15] The process of any one of [Embodiments 7-14], wherein the suspension stabilizer comprises a sugar, sugar alcohol, or sugar derivative.

[Embodiment 16] The process of any one of [Embodiments 7-15], wherein the suspension stabilizer comprises sorbitol or sucrose.

[Embodiment 17] The process of [Embodiment 8], wherein the solution comprises water, hydroxypropyl methylcellulose, sodium lauryl sulfate, and sucrose.

[Embodiment 18] The process of any one of [Embodiments 1-17], wherein the extragranular excipients comprise a lubricant, a disintegrant, a glidant, and a buffering agent.

[Embodiment 19] The process of [Embodiment 18], wherein the lubricant comprises lauric acid, myristic acid, palmitic acid, stearic acid or pharmaceutically acceptable salts or esters thereof, such as magnesium stearate, calcium stearate, sodium stearyl fumarate, or zinc stearate or a mixture thereof.

[Embodiment 20] The process of [Embodiment 18], wherein the lubricant comprises magnesium stearate.

[Embodiment 21] The process of any one of [Embodiments 18-20], wherein the disintegrant comprises low substituted hydroxypropyl cellulose, carboxymethylcellulose sodium, croscarmellose sodium, crospovidone, sodium starch glycolate or a mixture thereof.

[Embodiment 22] The process of any one of [Embodiments 18-20], wherein the disintegrant comprises crospovidone.

[Embodiment 23] The process of any one of [Embodiments 18-22], wherein the glidant comprises talc, calcium phosphate, calcium silicate, magnesium silicate, magnesium trisilicate, silicon dioxide, colloidal silicon dioxide, magnesium aluminosilicate, or a mixture thereof.

[Embodiment 24] The process of any one of [Embodiments 18-22], wherein the glidant comprises silicon dioxide.

[Embodiment 25] The process of any one of [Embodiments 18-24], wherein the buffering agent comprises sodium succinate, sodium citrate, sodium glutamate, sodium acetate, sodium lactate, or a mixture thereof.

[Embodiment 26] The process of any one of [Embodiments 18-24], wherein the buffering agent comprises trisodium citrate.

[Embodiment 27] The process of any one of [Embodiments 1-17], wherein the extragranular excipients comprise magnesium stearate, crospovidone, silicon dioxide, and trisodium citrate.

[Embodiment 28] The process of any one of [Embodiments 2-17], wherein the suspension is granulated with one or more solid carriers.

[Embodiment 29] The process of [Embodiment 28], wherein each solid carrier is selected from the group consisting of a saccharide, a disaccharide, a sugar alcohol, a polysaccharide, and a polysaccharide derivative.

[Embodiment 30] The process of [Embodiment 28], wherein each solid carrier is selected from the group consisting of a saccharide, a disaccharide, a sugar alcohol, a polysaccharide, and a polysaccharide derivative.

[Embodiment 31] The process of [Embodiment 28], wherein each solid carrier is a disaccharide or a polysaccharide.

[Embodiment 32] The process of [Embodiment 28], wherein the solid carrier comprises lactose and microcrystalline cellulose.

[Embodiment 33] The process of any one of [Embodiments 1-32], further comprising filling a capsule shell with at least a portion of the blend.

[Embodiment 34] The process of [Embodiment 1-32], further comprising compressing at least a portion of the blend to provide a solid dosage.

[Embodiment 35] An oral solid pharmaceutical composition comprising a meloxicam micro-cocrystal or nano-cocrystal and one or more pharmaceutically acceptable excipients.

[Embodiment 36] The composition of [Embodiment 35], comprising a granulate and one or more pharmaceutically acceptable extragranular excipients, wherein the granulate comprises the meloxicam nano-cocrystal and one or more pharmaceutically acceptable intragranular excipients.

[Embodiment 37] The pharmaceutical composition of [Embodiment 36], wherein each excipient is independently selected from the group consisting of a polymeric binder, a surface stabilizer, a lubricant, a disintegrant, a glidant, and a buffering agent.

[Embodiment 38] The composition of [Embodiment 36], wherein the one or more intragranular excipients comprise a polymeric binder, a surface stabilizer, a suspension stabilizer, and a solid carrier.

[Embodiment 39] The composition of [Embodiment 37 or 38], wherein the polymeric binder comprises a hydrophilic polymer.

[Embodiment 40] The composition of [Embodiment 37 or 38], wherein the polymeric binder comprises polyvinylpyrrolidone, hydroxypropyl methylcellulose, or a mixture thereof.

[Embodiment 41] The composition of [Embodiment 37 or 38], wherein the polymeric binder comprises hydroxypropyl methylcellulose.

[Embodiment 42] The composition of any one of [Embodiments 37-41], wherein the surface stabilizer is a surfactant, a sugar, a sugar alcohol, or a sugar derivative (e.g., a surfactant).

[Embodiment 43] The composition of any one of [Embodiments 37-41], wherein the surface stabilizer comprises sodium lauryl sulfate, ammonium lauryl sulfate, docusate sodium, ammonium dinonyl sulfosuccinate, diamyl sulfosuccinate sodium, dicapryl sulfosuccinate sodium, diheptyl sulfosuccinate sodium, dihexyl sulfosuccinate sodium, diisobutyl sulfosuccinate sodium, ditridecyl sulfosuccinate sodium, sodium dodecylbenzenesulfonate, or a mixture thereof.

[Embodiment 44] The composition of any one of [Embodiments 37-41], wherein the surface stabilizer comprises sodium lauryl sulfate.

[Embodiment 45] The composition of any one of [Embodiments 37-44], wherein the suspension stabilizer comprises a sugar, sugar alcohol, or sugar derivative.

[Embodiment 46] The composition of any one of [Embodiments 37-44], wherein the suspension stabilizer comprises sorbitol or sucrose.

[Embodiment 47] The composition of any one of [Embodiments 37-46], wherein each solid carrier is selected from the group consisting of a saccharide, a disaccharide, a sugar alcohol, a polysaccharide, and a polysaccharide derivative.

[Embodiment 48] The composition of any one of [Embodiments 37-46], wherein each solid carrier is selected from the group consisting of a saccharide, a disaccharide, a sugar alcohol, a polysaccharide, and a polysaccharide derivative.

[Embodiment 49] The composition of any one of [Embodiments 37-46], wherein each solid carrier is a disaccharide or a polysaccharide.

[Embodiment 50] The composition of any one of [Embodiments 37-46], wherein the solid carrier comprises lactose and microcrystalline cellulose.

[Embodiment 51] The composition of any one of [Embodiments 36-50], wherein the extragranular excipients comprise a lubricant, a disintegrant, a glidant, and a buffering agent.

[Embodiment 52] The composition of [Embodiment 37 or 51], wherein the lubricant comprises lauric acid, myristic acid, palmitic acid, stearic acid or pharmaceutically acceptable salts or esters thereof, such as magnesium stearate, calcium stearate, sodium stearyl fumarate, or zinc stearate or a mixture thereof.

[Embodiment 53] The composition of [Embodiment 37 or 51], wherein the lubricant comprises magnesium stearate.

[Embodiment 54] The composition of any one of [Embodiments 37 and 51-53], wherein the disintegrant comprises low substituted hydroxypropyl cellulose, carboxymethylcellulose sodium, croscarmellose sodium, crospovidone, and sodium starch glycolate, or a mixture thereof.

[Embodiment 55] The composition of any one of [Embodiments 37 and 51-53], wherein the disintegrant comprises crospovidone.

[Embodiment 56] The composition of any one of [Embodiments 37 and 51-55], wherein the glidant comprises talc, calcium phosphate, calcium silicate, magnesium silicate, magnesium trisilicate, silicon dioxide, colloidal silicon dioxide, magnesium aluminosilicate, or a mixture thereof.

[Embodiment 57] The composition of any one of [Embodiments 37 and 51-55], wherein the glidant comprises silicon dioxide.

[Embodiment 58] The composition of any one of [Embodiments 37 and 51-57], wherein the buffering agent comprises sodium succinate, sodium citrate, sodium glutamate, sodium acetate, sodium lactate, or a mixture thereof.

[Embodiment 59] The composition of any one of [Embodiments 37 and 51-57], wherein the buffering agent comprises trisodium citrate.

[Embodiment 60] An oral solid composition comprising a meloxicam co-crystal and one or pharmaceutically acceptable excipients, wherein the composition comprises an amount of a meloxicam co-crystal (e.g., equivalent to about 1 mg to about 60 mg, or 5 mg, 10 mg, or 15 mg meloxicam base), and the meloxicam is released in 900 mL of 0.1N HCl as measured by a USP-II Apparatus at 75 rpm and 37±2° C. according to one of: greater than about 30 wt. %, about 40 wt. %, about 50 wt. %, about 60 wt. %, or about 70 wt. % at 60 minutes; greater than about 30 wt. %, about 40 wt. %, about 50 wt. %, or about 60 wt. % at 30 minutes; or greater than about 30 wt. %, about 40 wt. %, or about 50 wt. % at 15 minutes.

[Embodiment 61] An oral solid composition comprising a meloxicam co-crystal and a pharmaceutically acceptable excipient, wherein the composition comprises an amount of a meloxicam co-crystal (e.g., equivalent to about 1 mg to about 60 mg, or 5 mg, 10 mg, or 15 mg meloxicam base), and the meloxicam is released in 900 mL of an acetate buffer as measured in a USP-II Apparatus at 75 rpm and 37±2° C. according to one of: greater than about 30 wt. %, about 40 wt. %, about 50 wt. %, about 60 wt. %, or about 70 wt. %, or about 80 wt. % at 60 minutes; greater than about 30 wt. %, about 40 wt. %, about 50 wt. %, or about 60 wt. %, or about 70 wt. % at 30 minutes; or greater than about 30 wt. %, about 40 wt. %, or about 50 wt. %, or about 60 wt. % at 15 minutes.

[Embodiment 62] An oral solid composition comprising a meloxicam co-crystal and a pharmaceutically acceptable excipient, wherein the composition comprises an amount of a meloxicam co-crystal (e.g., equivalent to about 1 mg to about 60 mg, or 5 mg, 10 mg, or 15 mg meloxicam base), and having one or more of:

| | PK parameter | Range (between about) | | or A:MOBIC tablets |
|---|---|---|---|---|
| (a) | Cₘₐₓ | 1500-3000 | ng/mL | or 1.2-2.0 times; |
| (b) | pAUC(0-1) | 400-1200 | ng · h/mL | or 2.5-7.0 times; |
| (c) | pAUC(0-2) | 1200-3500 | ng · h/mL | or 2.0-5.5 times; |
| (d) | pAUC(0-3) | 2500-5500 | ng · h/mL | or 1.3-5.0 times; |
| (e) | pAUC(0-4) | 4000-7500 | ng · h/mL | or 1.2-3.2 times; |
| (f) | pAUC(0-6) | 7000-12000 | ng · h/mL | or 1.1-2.5 times; |
| (g) | median Tₘₐₓ | 1.5-3.0 | hours; | |

or a bioequivalent thereof, where each of the PK ranges is measured in the same cohort of fasting subjects in a single-dose pharmacokinetic study or a post-surgery dental pain study, and where “A: MOBIC tablets” is the geometric least squares mean ratio of the referenced natural log-transformed PK parameter for the oral solid composition to MOBIC tablets in the same cohort of fasting subjects in a single-dose pharmacokinetic study.

[Embodiment 63] The oral solid composition of [Embodiment 60 or 61], characterized by one or more of:

| | PK parameter | Range (between about) | | or A:MOBIC tablets |
|---|---|---|---|---|
| (a) | Cₘₐₓ | 1500-3000 | ng/mL | or 1.2-2.0 times; |
| (b) | pAUC(0-1) | 400-1200 | ng · h/mL | or 2.5-7.0 times; |
| (c) | pAUC(0-2) | 1200-3500 | ng · h/mL | or 2.0-5.5 times; |
| (d) | pAUC(0-3) | 2500-5500 | ng · h/mL | or 1.3-5.0 times; |
| (e) | pAUC(0-4) | 4000-7500 | ng · h/mL | or 1.2-3.2 times; |
| (f) | pAUC(0-6) | 7000-12000 | ng · h/mL | or 1.1-2.5 times; |
| (g) | median Tₘₐₓ | 1.5-3.0 | hours; | |

or a bioequivalent thereof, where each of the PK ranges is measured in the same cohort of fasting subjects in a single-dose pharmacokinetic study or a post-surgery dental pain study, and where “A: MOBIC tablets” is the geometric least squares mean ratio of the referenced natural log-transformed PK parameter for the oral solid composition to MOBIC tablets in the same cohort of fasting subjects in a single-dose pharmacokinetic study.

[Embodiment 64] The oral solid composition of [Embodiment 62 or 63], wherein the meloxicam co-crystal is selected from the group consisting of meloxicam: succinic acid (2:1); meloxicam: aspirin (1:1), meloxicam: xinafoic acid (1:1), meloxicam: salicylic acid (1:1), and meloxicam: maleic acid (1:1).

[Embodiment 65] The oral solid composition of any one of [Embodiments 62-64], wherein the meloxicam co-crystal is meloxicam: succinic acid (2:1).

[Embodiment 66] The oral solid composition of [Embodiment 65], wherein the Cₘₐₓ is one of:

| | PK parameter | Range (between about) | or A:MOBIC tablets |
|---|---|---|---|
| (a) | Cₘₐₓ | 1800-2500 ng/mL; | or 1.3-1.7 times; or |
| (b) | Cₘₐₓ | 2000-2400 ng/mL; | or 1.4-1.6 times. |

[Embodiment 67] The oral solid composition of [Embodiment 65 or 66], wherein the median Tₘₐₓ is between about 1.5-3.0 hours or 2.0 and 3.0 hours.

[Embodiment 68] The oral solid composition of any one of [Embodiments 65-67], wherein the pAUC(0-2) is one of:

| | PK parameter | Range (between about) | or A:MOBIC tablets |
|---|---|---|---|
| (a) | pAUC(0-2) | 1800-2600 ng · h/mL; | or 2.0-3.5 times; or |
| (b) | pAUC(0-2) | 2000-2400 ng · h/mL; | or 2.0-3.0 times. |

[Embodiment 69] The oral solid composition of any one of [Embodiments 65-68], wherein pAUC(0-4) is one of:

| | PK parameter | Range (between about) | or A:MOBIC tablets |
|---|---|---|---|
| (a) | pAUC(0-4) | 4000-7000 ng · h/mL; | or 1.2-2.3 times; or |
| (b) | pAUC(0-4) | 5200-6200 ng · h/mL; | or 1.4-2.0 times. |

[Embodiment 70] The oral solid composition of any one of [Embodiments 62-64], wherein the meloxicam co-crystal is meloxicam: xinafoic acid (1:1).

[Embodiment 71] The oral solid composition of [Embodiment 70, wherein the Cₘₐₓ is one of:

| | PK parameter | Range (between about) | or A:MOBIC tablets |
|---|---|---|---|
| (a) | Cₘₐₓ | 1600-2600 ng/mL; | or 1.3-2.0 times; or |
| (b) | Cₘₐₓ | 2000-2400 ng/mL; | or 1.3-1.8 times. |

[Embodiment 72] The oral solid composition of [Embodiment 70 or 71], wherein the median Tₘₐₓ is between about 1.5-3.0 hours or 2.0 and 3.0 hours. [Embodiment 73] The oral solid composition of any one of [Embodiments 70-72], wherein the pAUC(0-2) is one of

| | PK parameter | Range (between about) | or A:MOBIC tablets |
|---|---|---|---|
| (a) | pAUC(0-2) | 1200-3000 ng · h/mL; | or 2.2-5.0 times; or |
| (b) | pAUC(0-2) | 1300-2000 ng · h/mL; | or 2.5-4.0 times. |

[Embodiment 74] The oral solid composition of any one of [Embodiments 70-73], wherein pAUC(0-4) is one of:

| | PK parameter | Range (between about) | or A:MOBIC tablets |
|---|---|---|---|
| (a) | pAUC(0-4) | 4000-7000 ng · h/mL; | or 1.6-3.0 times; or |
| (b) | pAUC(0-4) | 4500-5500 ng · h/mL; | or 1.7-2.6 times. |

[Embodiment 75] The oral solid composition of any one of [Embodiments 62-64], wherein the meloxicam co-crystal is meloxicam: salicylic acid (1:1).

[Embodiment 76] The oral solid composition of [Embodiment 75], wherein the Cₘₐₓ is one of:

| | PK parameter | Range (between about) | or A:MOBIC tablets |
|---|---|---|---|
| (a) | Cₘₐₓ | 1500-3000 ng/mL; | or 1.2-2.0 times; or |
| (b) | Cₘₐₓ | 1600-2800 ng/mL; | or 1.3-1.8 times. |

[Embodiment 77] The oral solid composition of [Embodiment 75 or 76], wherein the median Tₘₐₓ is between about 1.5 and 3.0 hours or 1.5-2.5 hours.

[Embodiment 78] The oral solid composition of any one of [Embodiments 75-77], wherein the pAUC(0-2) is one of:

| | PK parameter | Range (between about) | or A:MOBIC tablets |
|---|---|---|---|
| (a) | pAUC(0-2) | 1200-3500 ng · h/mL; | or 2.5-5.5 times; or |
| (b) | pAUC(0-2) | 1500-3000 ng · h/mL; | or 2.8-5.0 times. |

[Embodiment 79] The oral solid composition of any one of [Embodiments 75-78], wherein pAUC(0-4) is one of:

| | PK parameter | Range (between about) | or A:MOBIC tablets |
|---|---|---|---|
| (a) | pAUC(0-4) | 3500-7500 ng · h/mL; | or 1.6-3.2 times; or |
| (b) | pAUC(0-4) | 4000-7000 ng · h/mL; | or 1.8-3.0 times. |

[Embodiment 80] An oral solid composition comprising an amount of a meloxicam co-crystal (e.g., equivalent to about 1 mg to about 60 mg, or 5 mg, 10 mg, or 15 mg meloxicam base), and a pharmaceutically acceptable excipient, wherein the composition provides at least a therapeutic blood plasma concentration of meloxicam in less than about 3 hours as measured in a single-dose pharmacokinetic study or a post-surgical dental pain study

[Embodiment 81] The oral solid composition of [Embodiment 80], wherein the therapeutic blood plasma concentration of meloxicam is about 1000 ng/mL or a bioequivalent thereof.

[Embodiment 82] The oral solid composition of [Embodiment 80], wherein the therapeutic blood plasma concentration of meloxicam is about 1200 ng/mL or a bioequivalent thereof.

[Embodiment 83] The oral solid composition of [Embodiment 80], wherein the therapeutic blood plasma concentration of meloxicam is between about 1000 ng/mL and about 3000 ng/mL or a bioequivalent thereof.

[Embodiment 84] The oral solid composition of [Embodiment 80], wherein the therapeutic blood plasma concentration of meloxicam is between about 1200 ng/mL and about 2500 ng/mL or a bioequivalent thereof.

[Embodiment 85] The oral solid composition of any one of [Embodiments 80-84], wherein the composition provides at least the therapeutic blood plasma concentration of meloxicam in less than about 2.5 hours.

[Embodiment 86] The oral solid composition of any one of [Embodiments 80-84], wherein the composition provides at least the therapeutic blood plasma concentration of meloxicam in between about 0.5 hour and 4 hours.

[Embodiment 87] The oral solid composition of any one of [Embodiments 80-86], wherein the therapeutically effective amount of meloxicam co-crystal is an amount equivalent to between about 5 mg and 25 mg meloxicam base.

[Embodiment 88] The oral solid composition of [Embodiment 87], wherein the therapeutically effective amount of meloxicam co-crystal is an amount equivalent to between about 5 mg and 15 mg meloxicam base (e.g., equivalent to 10 mg or 15 mg meloxicam base).

[Embodiment 89] The oral solid composition of any one of [Embodiments 80-88], wherein the meloxicam co-crystal is selected from the group consisting of meloxicam: succinic acid (2:1);

meloxicam: aspirin (1:1), meloxicam: xinafoic acid (1:1), meloxicam: salicylic acid (1:1), and meloxicam: maleic acid (1:1).

[Embodiment 90] The oral solid composition of [Embodiment 89], wherein the meloxicam co-crystal is meloxicam: succinic acid (2:1).

[Embodiment 91] The oral solid composition of [Embodiment 89], wherein the meloxicam co-crystal is meloxicam: aspirin (1:1).

[Embodiment 92] The oral solid composition of [Embodiment 89], wherein the meloxicam co-crystal is meloxicam: xinafoic acid (1:1).

[Embodiment 93] The oral solid composition of [Embodiment 89], wherein the meloxicam co-crystal is meloxicam: salicylic acid (1:1).

[Embodiment 94] The oral solid composition of [Embodiment 89], wherein the meloxicam co-crystal is meloxicam: maleic acid (1:1).

[Embodiment 95] The oral solid composition of any one of [Embodiment 80-94], wherein the amount of meloxicam co-crystal is suitable for the treatment of pain via one or twice daily administration.

[Embodiment 96] The oral solid composition of any one of [Embodiment 80-94], wherein the amount of the meloxicam co-crystal is suitable for the treatment of acute pain via one or twice daily administration.

[Embodiment 97] The oral solid composition of any one of [Embodiment 80-96], wherein the oral solid composition provides a mean time of first perceptible relief of between about 5 minutes and about 60 minutes as measured in a post-surgical dental pain study.

[Embodiment 98] The oral solid composition of any one of [Embodiment 80-96], wherein the oral solid composition provides a mean time of meaningful relief between about 15 minutes and about 90 minutes as measured in a post-surgical dental pain study.

[Embodiment 99] A method for treating pain or acute pain comprising administering to a person in need of such treatment an oral solid composition according to any one of [Embodiment 1-98].

[Embodiment 100] The method of [Embodiment 99], wherein the administering is once daily administration.

[Embodiment 101] The method of [Embodiment 99], wherein the administering is twice daily administration.

[Embodiment 102] The method of any one of [Embodiment 99-101], wherein the person in need of such treatment is a person diagnosed with an opioid use disorder.

[Embodiment 103] The method of any one of [Embodiment 99-102], wherein the pain is acute pain.

[Embodiment 104] The method of [Embodiment 103], wherein the acute pain is post-surgical pain (e.g., post-molar extraction pain).

[Embodiment 105] The method of any one of [Embodiment 99-104], wherein the oral solid composition is administered to a person in need of such treatment and having an empty stomach.

[Embodiment 106] The method of [Embodiment 99-105], wherein the pain is post-surgical pain and the oral solid composition provides a reduction of rescue medication use of at least 10% as compared to patients receiving placebo as measured in a post-surgical dental pain study.

[Embodiment 107] The method of [Embodiment 106], wherein the reduction of rescue medication use is at least 25%.

[Embodiment 108] The method of [Embodiment 106], wherein the reduction of rescue medication use is at least 50%.

[Embodiment 109] The method of [Embodiment 106], wherein the reduction of rescue medication use is at least 75%.

[Embodiment 1a] A method for treating acute pain comprising administering to a person in need of such treatment an oral solid pharmaceutical composition comprising a meloxicam co-crystal, including any oral solid pharmaceutical composition of any one of [Embodiments 35-98].

[Embodiment 2a] The method of [Embodiment 1a], wherein the acute pain is post-surgical pain.

[Embodiment 3a] The method of [Embodiment 1a or 2a], wherein the meloxicam co-crystal is a meloxicam nano-cocrystal.

[Embodiment 4a] The method of any one of [Embodiments 1a-3a], wherein the pain is post-surgical pain and the oral solid composition provides a reduction of rescue medication use as compared to patients receiving placebo tablets as measured in a post-surgical dental pain study.

[Embodiment 5a] The method of any one of [Embodiments 1a-4a], wherein the oral solid composition comprises an amount of meloxicam co-crystal equivalent to between about 1 mg and 60 mg (e.g., 5 mg and 30 mg) meloxicam base.

[Embodiment 6a] The method of any one of [Embodiments 1a-5a], wherein the oral solid composition provides a reduced mean time of first perceptible relief and/or a reduced mean time of meaningful relief as compared to placebo tablets as measured in a post-surgical dental pain study.

[Embodiment 7a] The method of any one of [Embodiments 1a-6a], wherein the administering is once daily administration.

[Embodiment 8a] The method of any one of [Embodiments 1a-6a], wherein the administering is twice daily administration.

[Embodiment 9a] The method of any one of [Embodiments 1a-8a], wherein the person in need of such treatment is a person diagnosed with an opioid use disorder.

[Embodiment 10a] The method of any one of [Embodiments 1a-9a], wherein the meloxicam co-crystal is selected from the group consisting of meloxicam: succinic acid (2:1); meloxicam: aspirin (1:1), meloxicam: xinafoic acid (1:1), meloxicam: salicylic acid (1:1), and meloxicam: maleic acid (1:1).

[Embodiment 11a] The method of [Embodiment 10a], wherein the meloxicam co-crystal is meloxicam: salicylic acid (1:1).

[Embodiment 12a] The method of any one of [Embodiments 1a-11a], wherein the oral solid composition provides a therapeutic blood plasma concentration of meloxicam in less than about 3 hours as measured in as measured in a post-surgical dental pain study or a single-dose pharmacokinetic study.

[Embodiment 13a] The method of [Embodiment 12a], wherein the oral solid composition provides the therapeutic blood plasma concentration of meloxicam in between about 0.5 hour and 3 hours.

[Embodiment 14a] The method of [Embodiment 13a], wherein the oral solid composition provides the therapeutic blood plasma concentration of meloxicam in less than about 2.5 hours.

[Embodiment 15a] The method of any one of [Embodiments 1a-14a], wherein the oral solid pharmaceutical composition contains an amount of meloxicam co-crystal equivalent to about 10 mg or about 15 mg meloxicam base and provides a blood plasma concentration of meloxicam of between 500-2500 ng/mL, or a bioequivalent thereof, in less than about 2 hours, as measured in a single-dose pharmacokinetic study or a post-surgical dental pain study.

[Embodiment 16a] The method of any one of [Embodiments 1a-14a], wherein the oral solid pharmaceutical composition contains an amount of meloxicam co-crystal equivalent to about 10 mg or about 15 mg meloxicam base and provides a blood plasma concentration of meloxicam of: (a) between 1000-2500 ng/mL, or a bioequivalent thereof, in less than about 2 hours; and (b) between 500-1500 ng/mL, or a bioequivalent thereof, in less than about 0.5 hour; as measured in a single-dose pharmacokinetic study or a post-surgical dental pain study.

[Embodiment 17a] The method of [Embodiment 15a], wherein the oral solid pharmaceutical composition contains an amount of meloxicam co-crystal equivalent to about 15 mg meloxicam base.

[Embodiment 18a] The method of [Embodiment 17a], wherein the oral solid composition provides a blood plasma concentration of meloxicam of: (a) between 1000-2500 ng/mL, or a bioequivalent thereof, in less than about 2 hours; and (b) between 500-1500 ng/mL, or a bioequivalent thereof, in less than about 0.5 hour; as measured in a single-dose pharmacokinetic study or a post-surgical dental pain study.

[Embodiment 19a] The method of [Embodiment 15a], wherein the oral solid pharmaceutical composition contains an amount of meloxicam co-crystal equivalent to about 10 mg meloxicam base and provides a blood plasma concentration of between 500-1500 ng/mL, or a bioequivalent thereof, in less than about 2 hours, as measured in a single-dose pharmacokinetic study or a post-surgical dental pain study.

[Embodiment 20a] The method of [Embodiment 19a], wherein the oral solid pharmaceutical composition provides a blood plasma concentration of meloxicam of: (a) between 1000-1500 ng/mL, or a bioequivalent thereof, in less than about 2 hours; and (b) between 500-1000 ng/mL, or a bioequivalent thereof, in less than about 0.5 hour; as measured in a single-dose pharmacokinetic study or a post-surgical dental pain study.

The following examples are merely representative and are not intended to limit the scope of the present disclosure. It will be apparent to one skilled in the art that varying substitutions and modifications may be made to this disclosure without departing from the spirit thereof.

### EXAMPLES

### Example 1. Meloxicam: Xinafoic Acid Co-Crystal (1:1) Formulation

| | mg/tablet | % w/w | mg/tablet | % w/w |
|---|---|---|---|---|
| Name of ingredient | 7.5 mg base | | 15 mg base | |
| Drug Suspension | | | | |
| Meloxicam:Xinafoic acid | 7.50* | 7.50 | 15.00* | 7.50 |
| co-crystal (1:1) | | | | |
| Hypromellose E3 LV | 2.50 | 2.50 | 5.00 | 2.50 |
| (hydroxypropyl | | | | |
| methylcellulose) | | | | |
| sodium lauryl sulphate | 0.25 | 0.25 | 0.50 | 0.25 |
| sucrose | 7.50 | 7.50 | 15.00 | 7.50 |
| water | q.s. | | q.s. | |
| Intragranular Part | | | | |
| lactose monohydrate | 12.5 | 12.5 | 25.00 | 12.5 |
| microcrystalline cellulose | 60.75 | 60.75 | 121.50 | 60.75 |
| Extragranular Part | | | | |
| trisodium citrate dihydrate | 2.50 | 2.50 | 5.00 | 2.50 |
| crospovidone | 5.00 | 5.00 | 10.00 | 5.00 |
| colloidal silicon dioxide | 0.75 | 0.75 | 1.50 | 0.75 |
| magnesium stearate | 0.75 | 0.75 | 1.50 | 0.75 |
| Total Tablet Weight | 100.00 | 100.00 | 200.00 | 100.00 |

*meloxicam:xinafoic acid (1:1) co-crystal equivalent to 7.5 mg and 15 mg of meloxicam base, respectively

### Brief Manufacturing Process:

A dispersion of meloxicam: xinafoic acid (1:1) co-crystals with Hypromellose E3 LV, sodium lauryl sulphate and sucrose was prepared in purified water and stirred to get a uniform suspension. The uniform dispersion was homogenized and nanomilled until a particle size of less than 2 micron (2000 nm) was obtained. The nanomilled drug slurry was adsorbed by spraying on lactose monohydrate, microcrystalline cellulose mixture in a fluidized bed granulator. The resulting granules were dried and blended with extragranular ingredients. This final mixture was sized, lubricated, and compressed into tablets

### Example 2. Meloxicam: Succinic Acid Co-Crystal (2:1) Formulation

| | mg/tablet | % w/w | mg/tablet | % w/w |
|---|---|---|---|---|
| Name of ingredient | 7.5 mg base | | 15 mg base | |
| Drug Suspension | | | | |
| Meloxicam:Succinic Acid | 7.50* | 7.50 | 15.00* | 7.50 |
| Co-crystal (2:1) | | | | |
| Hypromellose E3 LV | 2.50 | 2.50 | 5.00 | 2.50 |
| (hydroxypropyl | | | | |
| methylcellulose) | | | | |
| sodium lauryl sulphate | 0.25 | 0.25 | 0.50 | 0.25 |
| sucrose | 7.50 | 7.50 | 15.00 | 7.50 |
| water | q.s. | | q.s. | |
| Intragranular Part | | | | |
| lactose monohydrate | 12.5 | 12.5 | 25.00 | 12.5 |
| microcrystalline cellulose | 60.75 | 60.75 | 121.50 | 60.75 |
| Extragranular Part | | | | |
| trisodium citrate dihydrate | 2.50 | 2.50 | 5.00 | 2.50 |
| crospovidone XL | 5.00 | 5.00 | 10.00 | 5.00 |
| colloidal silicon dioxide | 0.75 | 0.75 | 1.50 | 0.75 |
| magnesium stearate | 0.75 | 0.75 | 1.50 | 0.75 |
| Total Tablet Weight | 100.00 | 100.00 | 200.00 | 100.00 |

*meloxicam:succinic acid (2:1) co-crystal equivalent to 7.5 mg and 15 mg of meloxicam base, respectively

The formulation with meloxicam succinic acid co-crystal (2:1) was prepared using the materials and amounts in the preceding table according to the process of Example 1.

### Example 3. Meloxicam: Aspirin Co-Crystal (1:1) Formulation

| | |
|---|---|
| mg/tablet | % w/w |

| | |
|---|---|
| Name of ingredient | 15 mg base |

| | | |
|---|---|---|
| Drug Suspension | | |
| Meloxicam:Aspirin | 22.690 | 10.80 |
| co-crystal (1:1)* | | |
| Hypromellose E3 LV | 10.000 | 4.76 |
| Sodium Lauryl Sulphate | 0.500 | 0.24 |
| Sorbitol | 25.000 | 11.90 |
| Hydrochloric Acid | q.s | — |
| Water | q.s | — |
| Intragranular Part | | |
| Lactose Monohydrate | 25.000 | 11.90 |
| Microcrystalline Cellulose | 44.810 | 21.34 |
| Pregelatinized Starch | 52.500 | 25.00 |
| Extragranular Part | | |
| Sodium Citrate | 5.000 | 2.38 |
| Crospovidone XL | 20.000 | 9.52 |
| Colloidal silicon dioxide | 1.500 | 0.71 |
| Stearic Acid | 3.000 | 1.43 |
| Total Tablet Weight | 210.000 | 100.00 |

*meloxicam:aspirin co-crystal (1:1) equivalent to 15 mg meloxicam base.

The pH of the required quantity of water was adjusted to between 2.0-3.0 with hydrochloric acid. Hypromellose E3 LV, sorbitol, and sodium lauryl sulphate were dissolved in the preceding acidified water under stirring and the meloxicam co-crystal was added under stirring to get uniform suspension. The suspension was wet milled using a Nano mill for about suitable period to yield a drug suspension.

Lactose monohydrate, pregelatinized starch, microcrystalline cellulose were sifted through a suitable sieve, loaded in a fluid bed processor, and granulated with the preceding drug suspension The granules were dried and sifted through a suitable sieve.

Sodium citrate, crospovidone XL, colloidal silicon dioxide were sifted through a suitable sieve. The preceding granules were combined with these sifted ingredients and blended for a suitable time. Separately, stearic acid was sifted through a suitable sieve. The sifted stearic acid was added and further blended for suitable time. The blend was finally compressed on a rotary compression machine.

### Example 4. Clinical Formulations (“Nanosized”)

| | Name of ingredient | | | | | |
|---|---|---|---|---|---|---|
| | Formulation # | | | | | |
| | (1) | | (2) | | (3) | |
| | mg/ | % | mg/ | % | mg/ | % |
| | tablet | w/w | tablet | w/w | tablet | w/w |
| Drug Suspension | 15 mg base | | 15 mg base | | 15 mg base | |
| Meloxicam:Succinic Acid | 17.520 | 8.76 | — | — | | |
| co-crystal (2:1)* | | | | | | |
| Meloxicam:Xinafoic Acid | — | — | 23.033 | 11.52 | — | — |
| co-crystal (1:1)* | | | | | | |
| Meloxicam:Salicylic Acid | — | — | — | — | 20.896 | 10.45 |
| co-crystal (1:1)* | | | | | | |
| Hypromellose E3 LV | 5.000 | 2.50 | 5.000 | 2.50 | 5.000 | 2.50 |
| Sodium Lauryl Sulphate | 0.500 | 0.25 | 0.500 | 0.25 | 0.500 | 0.25 |
| Sucrose | 15.000 | 7.50 | 15.000 | 7.50 | 15.000 | 7.50 |
| Water | q.s | — | q.s | — | q.s | — |
| Intragranular Part | | | | | | |
| Lactose Monohydrate | 25.000 | 12.50 | 25.000 | 12.50 | 25.000 | 12.50 |
| Microcrystalline Cellulose | 118.980 | 59.49 | 113.467 | 56.73 | 113.604 | 56.80 |
| Extragranular Part | | | | | | |
| Sodium Citrate | 5.000 | 2.50 | 5.000 | 2.50 | 5.000 | 2.50 |
| Crospovidone XL | 10.000 | 5.00 | 10.000 | 5.00 | 10.000 | 5.00 |
| Colloidal silicon dioxide | 1.500 | 0.75 | 1.500 | 0.75 | 1.500 | 0.75 |
| Magnesium Stearate | 1.500 | 0.75 | 1.500 | 0.75 | — | — |
| Stearic Acid | — | — | — | — | 3.500 | 1.75 |
| Total Tablet Weight | 200.000 | 100.00 | 200.00 | 100.00 | 200.00 | 100.00 |

*Quantity equivalent to 15 mg of meloxicam base.

Hypromellose E3 LV, Sucrose, Sodium Lauryl Sulphate were dissolved in purified water under stirring and meloxicam co-crystal added to get a uniform suspension. The suspension was wet milled using a Nano mill for a suitable period. Separately, lactose monohydrate and microcrystalline cellulose were sifted through a suitable sieve. The sifted mixture was loaded into a fluid bed processor and granulated using the meloxicam co-crystal drug suspension The resulting granules were dried to the desired loss on drying, and sifted through a suitable sieve. Extragranular excipients sodium citrate, crospovidone XL, and colloidal silicon dioxide were sifted through a suitable sieve. Separately, magnesium stearate or stearic acid were sifted through a suitable sieve. The granules prepared above were blended with the sifter extragranular excipient mixture. Following this first blending, sifted magnesium stearate or stearic acid was added to the mixture and blended for a suitable time to provide the final blend. Compression of the final blend with a rotary compression machine provided finished oral tablets.

### Example 5. Clinical Formulations (“Micronized”)

| | Name of ingredient | | | | | |
|---|---|---|---|---|---|---|
| | Formulation # | | | | | |
| | (4) | | (5) | | (6) | |
| | mg/ | % | mg/ | % | mg/ | % |
| | tablet | w/w | tablet | w/w | tablet | w/w |
| | 15 mg | | 15 mg | | 15 mg | |
| Intragranular Part | | | | | | |
| Meloxicam:Succinic Acid | 17.520 | 9.73 | — | — | — | — |
| co-crystal (2:1)* | | | | | | |
| Meloxicam:Xinafoic Acid | — | — | 23.033 | 12.80 | — | — |
| co-crystal (1:1)* | | | | | | |
| Meloxicam:Salicylic Acid | — | — | — | — | 20.896 | 11.61 |
| co-crystal (1:1)* | | | | | | |
| Lactose Monohydrate | 47.250 | 26.25 | 47.250 | 26.25 | 47.250 | 26.25 |
| Microcrystalline cellulose | 44.730 | 24.85 | 39.217 | 21.79 | 41.354 | 22.97 |
| Crospovidone XL | 6.000 | 3.33 | 6.000 | 3.33 | 6.000 | 3.33 |
| Povidone k 30 | 6.500 | 3.61 | 6.500 | 3.61 | 6.500 | 3.61 |
| Water | q.s | — | q.s | — | q.s | — |
| Extragranular Part | | | | | | |
| Microcrystalline Cellulose | 50.000 | 27.78 | 50.000 | 27.78 | 48.500 | 26.94 |
| Sodium Citrate | 5.000 | 2.78 | 5.000 | 2.78 | 5.000 | 2.78 |
| Colloidal silicon dioxide | 1.500 | 0.83 | 1.500 | 0.83 | 1.500 | 0.83 |
| Magnesium Stearate | 1.500 | 0.83 | 1.500 | 0.83 | — | — |
| Stearic Acid | — | — | — | — | 3.000 | 1.67 |
| Total Tablet Weight | 180.00 | 100.00 | 180.00 | 100.00 | 180.00 | 100.00 |

*Quantity equivalent to 15 mg of meloxicam base

Povidone K 30 was dissolved in purified water under stirring to form a clear solution. Separately, meloxicam co-crystal, lactose monohydrate, microcrystalline cellulose, and crospovidone XL were sifted through a suitable sieve. The sieved mixture was loaded into a rapid mixer granulator (RMG) and granulated using the povidone K 30 solution. The resulting granules were dried until the desired loss on drying was achieved, and then sifted through a suitable sieve. Extragranular excipients microcrystalline cellulose, sodium citrate, and colloidal silicon dioxide were sifted through a suitable sieve to give an excipient mixture. Separately stearic acid or magnesium sulfate (as noted in the preceding table) was sifted through a suitable sieve. The dried and sifted granules were combined with the sifted excipient mixture in a blender and blended for suitable time. The sifted stearic acid or magnesium stearate was added and blending continued. This final blend was compressed into tablets in a rotary compression machine.

### Example 6. Comparative Meloxicam: Aspirin Co-Crystal (1:1) Formulation (“Micronized”)

| | Approach | | | |
|---|---|---|---|---|
| | | | Non-aqueous Granulation | |
| | Aqueous Granulation | | (Non- [symbol] | |
| Name of ingredient | mg/tablet | % w/w | mg/tablet | % w/w |
| Intragranular Part | | | | |
| Meloxicam:Aspirin | 22.690 | 12.61 | 22.690 | 12.61 |
| co-crystal (1:1)* | | | | |
| Lactose Anhydrous | 30.000 | 16.67 | 30.000 | 16.67 |
| Pregelatinized Starch | 51.810 | 28.78 | 51.810 | 28.78 |
| Crospovidone XL | 12.000 | 6.67 | 12.000 | 6.67 |
| Povidone k 30 | 5.000 | 2.78 | 5.000 | 2.78 |
| Hydrochloric Acid | q.s | — | — | — |
| Water | q.s | — | — | — |
| Ethanol | — | — | q.s | — |
| Extragranular Part | | | | |
| Microcrystalline | 14.500 | 8.06 | 14.500 | 8.06 |
| Cellulose | | | | |
| Pregelatinized Starch | 28.000 | 15.56 | 28.000 | 15.56 |
| Crospovidone XL | 5.000 | 2.78 | 5.000 | 2.78 |
| Sodium Citrate | 5.000 | 2.78 | 5.000 | 2.78 |
| Colloidal silicon | 2.000 | 1.11 | 2.000 | 1.11 |
| dioxide | | | | |
| Stearic Acid | 4.000 | 2.22 | 4.000 | 2.22 |
| Total Tablet Weight | 180.000 | 100.00 | 180.000 | 100.00 |

*Meloxicam:aspirin (1:1) co-crystal equivalent to 15 mg of meloxicam base [symbol]
indicates data missing or illegible when filed

Aqueous granulation: A sufficient quantity of water was acidified with hydrochloric acid to adjust the pH to in between 2.0-3.0. Into this acidified water, povidone K 30 was dissolved under stirring to form a clear solution. Separately, meloxicam co-crystal, lactose anhydrous, pregelatinized starch, and crospovidone XL were sifted through a suitable sieve. The sieved mixture was loaded into a rapid mixer granulator (RMG) and granulated using the povidone K 30 solution. The resulting granules were dried until the desired loss on drying was achieved, and then sifted through a suitable sieve. Extragranular excipients microcrystalline cellulose, pregelatinized starch, sodium citrate, colloidal silicon dioxide, and crospovidone XL were sifted through a suitable sieve to give an excipient mixture. Separately stearic acid was sifted through a suitable sieve. The dried and sifted granules were combined with the sifted excipient mixture in a blender and blended for suitable time. The sifted stearic acid was added and blending continued. This final blend was compressed into tablets in a rotary compression machine.

Non-aqueous granulation: Povidone K 30 was dissolved in ethanol under stirring to form a clear solution. Separately, meloxicam co-crystal, lactose anhydrous, pregelatinized starch, and crospovidone XL were sifted through a suitable sieve. The sieved mixture was loaded into a rapid mixer granulator (RMG) and granulated using the ethanolic povidone K 30 solution. The resulting granules were dried until the desired loss on drying was achieved, and then sifted through a suitable sieve. Extragranular excipients microcrystalline cellulose, pregelatinized starch, sodium citrate, colloidal silicon dioxide, and crospovidone XL were sifted through a suitable sieve to give an excipient mixture. Separately stearic acid was sifted through a suitable sieve. The dried and sifted granules were combined with the sifted excipient mixture in a blender and blended for suitable time. The sifted stearic acid was added and blending continued. This final blend was compressed into tablets in a rotary compression machine.

### Example 7. Dissolution

Rate of API release (meloxicam) was measured in a USP Apparatus 2 (Paddle Apparatus) at 37+/−2° C., according to the methods of USP42-NF37 chapter (711) on dissolution, which is incorporated herein by reference. Release rate data collected for both “micronized” formulations (4)-(6) (Example 5) and “nanosized” formulations (1)-(3) (Example 4) for three different co-crystals in three different dissolution media, 0.1N HCl, acetate buffer (pH 4.5), and phosphate buffer (pH 6.1), as well as commercial MOBIC tablets and VIVLODEX capsules, are provided in Tables 2-4 and FIGS. 1a-c (succinic acid co-crystal, formulations (1) and (4)), 2a-c (xinafoic acid co-crystal, formulations (2) and (5)), and 3a-c (salicylic acid co-crystal, formulations (3) and (6)), respectively). Particle size profiles for the meloxicam co-crystal for each tested formulation is provided in Table 1. Since VIVLODEX capsules are not commercially available at 15 mg dosage strength, for an equal comparison, a gelatin capsule was filled with an amount of the VIVLODEX formulation containing 15 mg meloxicam base.

TABLE 1

| | | API (μm) | | |
|---|---|---|---|---|
| Co-crystal | Formulation# | D₉₀ | D₅₀ | D₁₀ |
| Meloxicam:Succinic Acid (2:1) | (1) | 1.57 | 0.16 | 0.07 |
| | (4) | 9.30 | 3.68 | 1.35 |
| Meloxicam:Xinafoic acid (1:1) | (2) | 0.71 | 0.44 | 0.15 |
| | (5) | 5.21 | 1.20 | 0.52 |
| Meloxicam:Salicylic Acid (1:1) | (3) | 0.50 | 0.14 | 0.07 |
| | (6) | 15.54 | 7.22 | 1.54 |

Surprisingly, both the micronized and nanosized formulation of each of the succinic acid and salicylic acid co-crystals exhibited substantially faster meloxicam release versus VIVLODEX and MOBIC comparators under acidic pH conditions (pH 4.5 and below) that an oral dosage form could experience following ingestion. While xinafoic acid co-crystals exhibited poor release under very low pH conditions, release acetate buffer also showed surprisingly faster release versus both VIVLODEX and MOBIC comparators. Under nearly neutral pH conditions (pH greater than about 6.1), the micronized and nanomilled formulations showed substantially improved release rates as compared to MOBIC tablets.

TABLE 2

| | MOBIC | VIVLODEX | Succinic acid co-crystal tablets | |
|---|---|---|---|---|
| Time | tabs | caps | Micronized | Nanomilled |
| (min) | 15 mg | 15 mg | (4) | (1) |
| | Medium: 0.1N HCl, | | | |
| | 900 mL, USP-II (Paddle), 75 rpm | | | |
| 5 | 1 | 10 | 35 | 41 |
| 10 | 3 | 13 | 41 | 50 |
| 15 | 3 | 14 | 43 | 55 |
| 30 | 5 | 15 | 43 | 58 |
| 45 | 6 | 15 | 41 | N/A |
| 60 | 6 | 16 | 39 | 57 |
| | Medium: pH 4.5 Acetate Buffer, | | | |
| | 900 mL, USP-II (Paddle), 75 rpm | | | |
| 5 | 4 | 13 | 45 | 49 |
| 10 | 6 | 18 | 58 | 57 |
| 15 | 7 | 19 | 66 | 61 |
| 30 | 9 | 19 | 74 | 67 |
| 45 | 10 | 19 | 78 | N/A |
| 60 | 11 | 20 | 80 | 69 |
| | Medium: pH 6.1 phosphate buffer, | | | |
| | 900 mL, USP-II(Paddle),75 rpm | | | |
| 5 | 20 | 86 | 82 | 90 |
| 10 | 26 | 91 | 89 | 91 |
| 15 | 31 | 93 | 92 | 97 |
| 30 | 45 | 94 | 95 | 100 |
| 60 | 58 | 95 | 97 | 100 |

TABLE 3

| | MOBIC | VIVLODEX | Xinafoic Acid co-crystal tablets | |
|---|---|---|---|---|
| Time | tabs | caps | Micronized | Nanomilled |
| (min) | 15 mg | 15 mg* | (5) | (2) |
| | Medium: 0.1N HCl, | | | |
| | 900 mL, USP-II (Paddle), 75 rpm | | | |
| 5 | 1 | 10 | 4 | 3 |
| 10 | 3 | 13 | 5 | N/A |
| 15 | 3 | 14 | 5 | 5 |
| 30 | 5 | 15 | 5 | 6 |
| 45 | 6 | 15 | 6 | 6 |
| 60 | 6 | 16 | 6 | 7 |
| | Medium: pH 4.5 Acetate Buffer, | | | |
| | 900 mL, USP-II (Paddle), 75 rpm | | | |
| 5 | 4 | 13 | 24 | 14 |
| 10 | 6 | 18 | 29 | 21 |
| 15 | 7 | 19 | 32 | 25 |
| 30 | 9 | 19 | 35 | 32 |
| 45 | 10 | 19 | 36 | 35 |
| 60 | 11 | 20 | 38 | 37 |
| | Medium: pH 6.1 phosphate buffer, | | | |
| | 900 mL, USP-II (Paddle), 75 rpm | | | |
| 5 | 20 | 86 | 83 | 77 |
| 10 | 26 | 91 | 90 | 94 |
| 15 | 31 | 93 | 92 | 98 |
| 30 | 45 | 94 | 94 | 98 |
| 60 | 58 | 95 | 95 | 99 |

TABLE 4

| | MOBIC | VIVLODEX | Salicylic Acid co-crystal tablets | |
|---|---|---|---|---|
| Time | tabs | caps | Micronized | Nanomilled |
| (min) | 15 mg | 15 mg* | (6) | (3) |
| | Medium: 0.1N HCl, | | | |
| | 900 mL, USP-II (Paddle), 75 rpm | | | |
| 5 | 1 | 10 | 33 | 28 |
| 10 | 3 | 13 | 53 | 48 |
| 15 | 3 | 14 | 61 | 55 |
| 30 | 5 | 15 | 72 | 59 |
| 45 | 6 | 15 | 76 | 60 |
| 60 | 6 | 16 | 74 | 61 |
| | Medium: pH 4.5 Acetate Buffer, | | | |
| | 900 mL, USP-II (Paddle), 75 rpm | | | |
| 5 | 4 | 13 | 70 | 61 |
| 10 | 6 | 18 | 80 | 62 |
| 15 | 7 | 19 | 84 | 63 |
| 30 | 9 | 19 | 85 | 65 |
| 45 | 10 | 19 | 87 | 66 |
| 60 | 11 | 20 | 88 | 68 |
| | Medium: pH 6.1 phosphate buffer, | | | |
| | 900 mL, USP-II (Paddle), 75 rpm | | | |
| 5 | 20 | 86 | 98 | 90 |
| 10 | 26 | 91 | 102 | 96 |
| 15 | 31 | 93 | 102 | 97 |
| 30 | 45 | 94 | 102 | 98 |
| 60 | 58 | 95 | 103 | 99 |

### Example 8. Single Dose Pharmacokinetic Studies

Randomized, three-period, three-treatment, three-sequence, crossover, balanced, single dose oral bioequivalence studies were performed to compare:

| | | | Reference |
|---|---|---|---|
| Study # | Test Product (T1) | Test Product (T2) | Product (R) |
| 1 | Meloxicam: Succinic | Meloxicam: Succinic | MOBIC |
| | acid (1:1) co-crystal, | acid (1:1) co-crystal, | (Meloxicam) |
| | micronized formulation | nanosized formulation | Tablets, |
| | (4), 15 mg | (1), 15 mg | 15 mg\hat{ } |
| 2 | Meloxicam: Xinafoic | Meloxicam: Xinafoic | |
| | acid (1:1) co-crystal, | acid (1:1) co-crystal, | |
| | micronized formulation | nanosized formulation | |
| | (5), 15 mg | (2), 15 mg | |
| 3 | Meloxicam: Salicylic | Meloxicam: Salicylic | |
| | acid (1:1) co-crystal, | acid (1:1) co-crystal, | |
| | micronized formulation | nanosized formulation | |
| | (6), 15 mg | (3), 15 mg | |

\hat{ }available from Boehringer Ingelheim Pharmaceuticals, Inc., Ridgefield, CT 06877 USA.

with the objective to compare and evaluate the oral bioavailability of test formulations of meloxicam co-crystal tablets 15 mg with that of MOBIC (Meloxicam) Tablets, 15 mg, in healthy, adult, human subjects under fasting conditions.

A cohort of 18 healthy, adult, South Asian male human subjects were tested. Each dosing interval was at least 7 days. Considering the minimum washout period, study duration of clinical part was 19 days from the day of check-in of first period.

Subjects were randomized into one of three sequences: T1T2R, T2RT1, or RT1T2. One tablet of either test product, T1 or T2, or reference product (R) was administered to the subjects as per the randomization schedule in a sitting posture with about 240 mL of water at ambient temperature in each period under the supervision of trained study personnel. All products were swallowed whole and not chewed, crushed or divided.

Fasting was required for at least 10 hours prior to dosing until at least 4 hours post-dose in each period. Water was restricted from at least 1 hour prior to dosing until at least 1 hour post-dose in each period (no fluid, except for water given with dosing). Subjects remained seated upright for initial 4 hours post-dose and only necessary movement was allowed during this period.

In each period, a total of 25 venous blood samples (4 mL each) were collected at pre-dose (0.0 hour) and at 0.167, 0.333, 0.5, 0.75, 1.0, 1.25, 1.5, 1.75, 2.0, 2.5, 3.0, 3.5, 4.0, 5.0, 6.0, 7.0, 8.0, 10.0, 12.0, 16.0, 24.0, 36.0, 48.0 and 72.0 hours post dose in labeled K₃EDTA containers.

After collection, the blood samples were chilled until the start of centrifugation. Blood samples were placed in a refrigerated centrifuge within 30 minutes of blood sample collection and then spun at 4000 rpm at 4° C.±2° C. for 10 minutes. The plasma was then separated, transferred to labeled polypropylene tubes in duplicates (approximately 1.0 mL plasma in primary aliquot and remaining volume in secondary aliquot) and stored in a freezer at −70° C.±20° C. at the clinical facility until shipment to an analytical facility. The samples were stored in a freezer at −70° C.±15° C. at the analytical facility until analyzed.

Plasma samples were assayed by validated method developed at the analytical facility, which is specific for the determination of meloxicam. The pharmacokinetic parameters Cₘₐₓ, AUCₜ, AUCᵢ, Tₘₐₓ, kₑᵢ, AUC_% Extrap_obs and t_{Half} were calculated using Phoenix® WinNonlin® professional software (Version 8.1 or higher). Statistical Analysis: Statistical analysis was performed on the pharmacokinetic parameters using SAS® statistical software (Version: 9.4 or higher; SAS Institute Inc, USA).

In analyzing the pharmacokinetic data, selected patients were excluded when subject blood plasma carryover shown in pre-dose samples for testing Periods 2 or 3 was greater than 5% Cₘₐₓ. Each study initiated with a cohort of 18 subjects, and the number of subjects included in each data set (N) is indicated in each of Tables 5-7.

TABLE 5
Succinic Acid Comparative Pharmacokinetic data (N = 17)

| | | Formulation | | | | | |
|---|---|---|---|---|---|---|---|
| | | | | | | MOBIC tablets, | |
| Parameter | | (1, Nano) | | (4, Micro) | | 15 mg | |
| AUC₀₋ₜ | (ng · h/mL) (CV %) | 50354.2 | (39.2%) | 50021.5 | (35.9%) | 43417.5 | (38.2%) |
| AUC_{0-inf} | (ng · h/mL) (CV %) | 57311.4 | (47.0%) | 56152.0 | (43.0%) | 50570.2 | (43.2%) |
| pAUC_{0-1 h} | (ng · h/mL) (CV %) | 887.6 | (81.3%) | 583.6 | (76.3%) | 222.2 | (69.8%) |
| pAUC_{0-2 h} | (ng · h/mL) (CV %) | 2221.4 | (59.8%) | 1881.6 | (60.9%) | 980.3 | (53.3%) |
| pAUC_{0-3 h} | (ng · h/mL) (CV %) | 3834.2 | (43.8%) | 3405.1 | (51.5%) | 2131.4 | (47.3%) |
| pAUC_{0-4 h} | (ng · h/mL) (CV %) | 5690.2 | (33.2%) | 5043.7 | (44.5%) | 3459.9 | (40.2%) |
| pAUC₀₋₆ | (ng · h/mL) (CV %) | 9073.9 | (25.3%) | 8419.8 | (29.5%) | 6121.8 | (30.9%) |
| Cₘₐₓ | (ng/mL) (CV %) | 2242.8 | (29.5%) | 2189.0 | (25.0%) | 1512.3 | (23.6%) |
| kₑₗ | (hr⁻¹) (CV %) | 0.0393 | (30.8%) | 0.0400 | (31.4%) | 0.0389 | (29.0%) |
| t_{1/2} | (hr) (CV %) | 19.38 | (32.5%) | 19.12 | (33.2%) | 19.30 | (28.9%) |
| Tₘₐₓ | (hr) median (range) | 3.0 | (0.33-5.0) | 2.5 | (0.75-5.0) | 4.0 | (1.5-12.0) |

TABLE 6
Xinafoic Acid Comparative Pharmacokinetic data (N = 15)

| | | Formulation | | | | | |
|---|---|---|---|---|---|---|---|
| | | | | | | MOBIC tablets, | |
| Parameter | | (2, Nano) | | (5, Micro) | | 15 mg | |
| AUC₀₋ₜ | (ng · h/mL) (CV %) | 54325.2 | (27.1%) | 53748.2 | (25.9%) | 50086.7 | (27.0%) |
| AUC_{0-inf} | (ng · h/mL) (CV %) | 64872.4 | (33.7%) | 62165.1 | (32.8%) | 59092.9 | (34.4%) |
| pAUC_{0-1 h} | (ng · h/mL) (CV %) | 568.7 | (93.1%) | 683.6 | (74.6%) | 170.7 | (98.1%) |
| pAUC_{0-2 h} | (ng · h/mL) (CV %) | 1690.0 | (66.8%) | 1887.3 | (61.3%) | 706.3 | (84.2%) |
| pAUC_{0-3 h} | (ng · h/mL) (CV %) | 3458.8 | (37.2%) | 3378.7 | (49.9%) | 1620.8 | (64.1%) |
| pAUC_{0-4 h} | (ng · h/mL) (CV %) | 5366.9 | (25.1%) | 5015.1 | (41.3%) | 2794.1 | (50.2%) |
| pAUC₀₋₆ | (ng · h/mL) (CV %) | 8691.2 | (18.1%) | 8217.5 | (26.5%) | 5347.5 | (32.7%) |
| Cₘₐₓ | (ng/mL) (CV %) | 2198.1 | (22.8%) | 2018.5 | (15.6%) | 1423.4 | (20.1%) |
| kₑₗ | (hr⁻¹) (CV %) | 0.0317 | (31.3%) | 0.0337 | (26.7%) | 0.0332 | (28.7%) |
| t_{1/2} | (hr) (CV %) | 23.98 | (32.2%) | 22.01 | (28.2%) | 22.70 | (30.6%) |
| Tₘₐₓ | (hr) median (range) | 2.5 | (0.5-4.0) | 2.5 | (0.33-5.0) | 4.0 | (3.0-10.0) |

TABLE 7
Salicylic Acid Comparative Pharmacokinetic data (N = 16)

| | | Formulation | | | | | |
|---|---|---|---|---|---|---|---|
| | | | | | | MOBIC tablets, | |
| Parameter | | (3, Nano) | | (6, Micro) | | 15 mg | |
| AUC₀₋ₜ | (ng · h/mL) (CV %) | 44813.9 | (27.0%) | 43927.3 | (20.9%) | 35967.6 | (19.7%) |
| AUC_{0-inf} | (ng · h/mL) (CV %) | 52315.1 | (33.9%) | 49270.2 | (28.0%) | 39525.0 | (22.4%) |
| pAUC_{0-1 h} | (ng · h/mL) (CV %) | 819.9 | (72.6%) | 796.1 | (50.5%) | 174.5 | (119.8%) |
| pAUC_{0-2 h} | (ng · h/mL) (CV %) | 2204.1 | (53.6%) | 2167.8 | (44.4%) | 649.4 | (92.9%) |
| pAUC_{0-3 h} | (ng · h/mL) (CV %) | 3802.6 | (39.2%) | 3737.5 | (34.0%) | 1451.2 | (75.2%) |
| pAUC_{0-4 h} | (ng · h/mL) (CV %) | 5514.6 | (30.0%) | 5479.5 | (23.7%) | 2499.5 | (61.4%) |
| pAUC₀₋₆ | (ng · h/mL) (CV %) | 8460.1 | (20.7%) | 8516.9 | (16.9%) | 4712.9 | (42.0%) |
| Cₘₐₓ | (ng/mL) (CV %) | 2063.5 | (15.0%) | 2020.4 | (13.4%) | 1292.9 | (24.6%) |
| kₑₗ | (hr⁻¹) (CV %) | 0.0330 | (25.7%) | 0.0341 | (21.3%) | 0.0374 | (16.6%) |
| t_{1/2} | (hr) (CV %) | 22.38 | (26.6%) | 21.29 | (24.2%) | 18.99 | (16.3%) |
| Tₘₐₓ | (hr) median (range) | 2.5 | (0.33-5.0) | 2.5 | (0.75-4.0) | 4.0 | (2.0-12.0) |

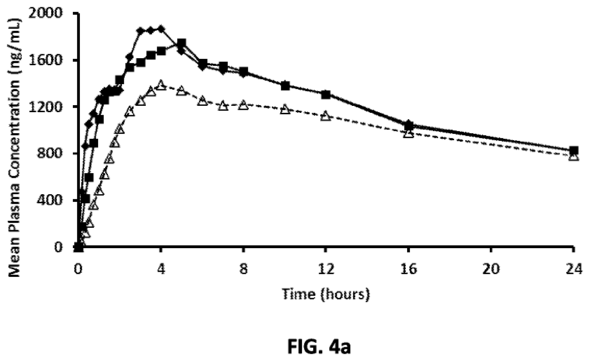

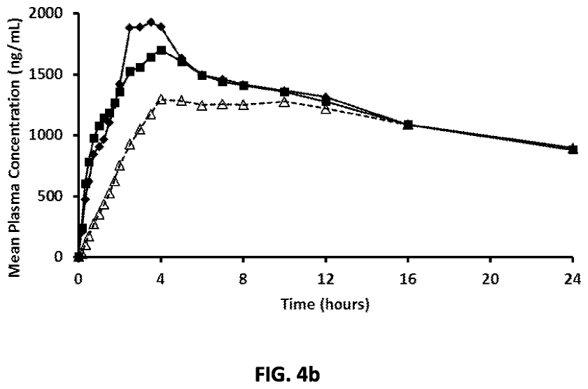

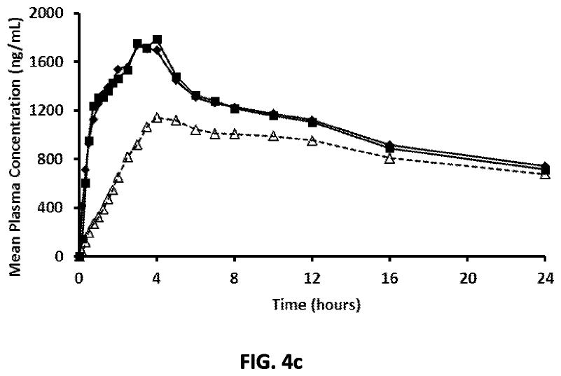

Mean plasma concentration of meloxicam for each of the formulations (1)-(6) as compared to MOBIC tablets in the same subject cohort are shown in FIGS. 4a-c. A review of the pharmacokinetic 5 data acquired in Studies 1-3 shows that for formulations (1)-(6) versus MOBIC tablets:
each exhibits an earlier Tₘₐₓ of about 2.5-3.0 hours versus 4.0 hours for MOBIC tablets;
each exhibits a higher Cₘₐₓ of about 2000 ng/mL to 2300 mg/mL versus 1250-1550 ng/mL for MOBIC tablets;
each exhibits more rapid systemic absorption as shown by pAUC(0-t) shown in Table 7.

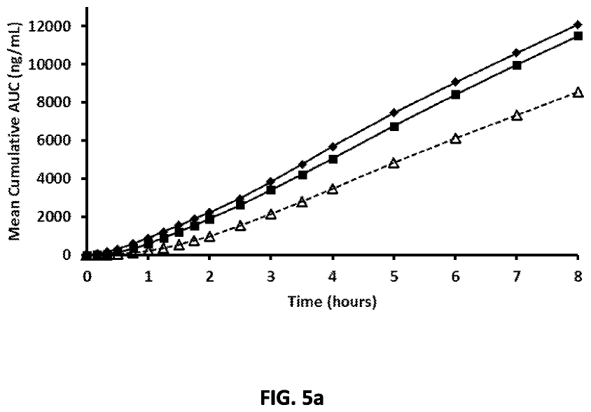

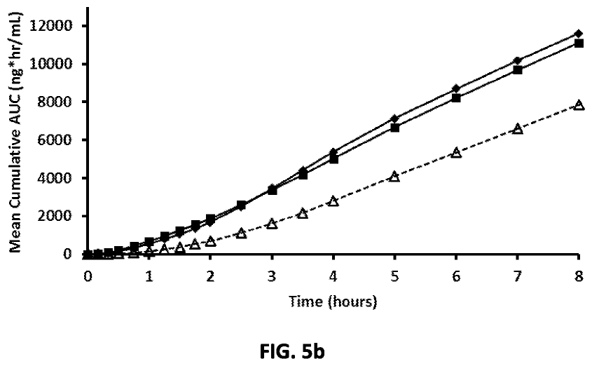

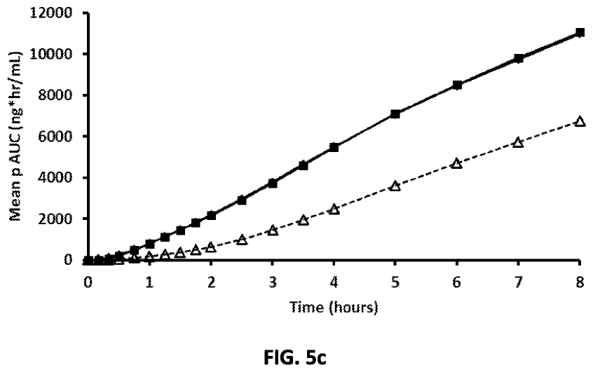

Plots of pAUC(0-t) for each of formulations (1)-(6) within the first eight hours after administration are provided in FIGS. 5a-c, illustrating the trends described above.

TABLE 8
Ratios of pharmacokinetic parameters for Formulations (1)-
(6) versus MOBIC tablets (R) (90% confidence intervals)

| Parameter | (1) | (2) | (3) | (4) | (5) | (6) |
|---|---|---|---|---|---|---|
| pAUC_{0-1 h} | 4.10\hat{ } | 3.19\hat{ } | 6.27\hat{ } | 2.61\hat{ } | 4.36\hat{ } | 6.80\hat{ } |
| (ng · h/mL) | (284\hat{ }%- | (190\hat{ }%- | (335\hat{ }%- | (180\hat{ }%- | (260\hat{ }%- | (370\hat{ }%- |
| | 593\hat{ }%) | 536\hat{ }%) | 1174\hat{ }%) | 377\hat{ }%) | 732\hat{ }%) | 1251\hat{ }%) |
| pAUC_{0-2 h} | 2.43\hat{ } | 2.59\hat{ } | 4.72\hat{ } | 1.93\hat{ } | 2.98\hat{ } | 4.75\hat{ } |
| (ng · h/mL) | (167\hat{ }%- | (162\hat{ }%- | (268\hat{ }%- | (133\hat{ }%- | (187\hat{ }%- | (275\hat{ }%- |
| | 353\hat{ }%) | 412\hat{ }%) | 829\hat{ }%) | 281\hat{ }%) | 475\hat{ }%) | 822\hat{ }%) |
| pAUC_{0-3 h} | 2.02\hat{ } | 2.56\hat{ } | 3.71\hat{ } | 1.63\hat{ } | 2.26\hat{ } | 3.67\hat{ } |
| (ng · h/mL) | (150\hat{ }%- | (178\hat{ }%- | (229\hat{ }%- | (121%- | (157\hat{ }%- | (230\hat{ }%- |
| | 273\hat{ }%) | 368\hat{ }%) | 600\hat{ }%) | 219\hat{ }%) | 325\hat{ }%) | 585\hat{ }%) |
| pAUC_{0-4 h} | 1.75\hat{ } | 2.16\hat{ } | 2.77\hat{ } | 1.43\hat{ } | 1.83\hat{ } | 2.80\hat{ } |
| (ng · h/mL) | (139\hat{ }%- | (164\hat{ }%- | (191\hat{ }%- | (114%- | (139\hat{ }%- | (196\hat{ }%- |
| | 221\hat{ }%) | 284*%) | 402\hat{ }%) | 181\hat{ }%) | 241\hat{ }%) | 402\hat{ }%) |
| pAUC_{0-6 h} | 1.51\hat{ } | 1.68\hat{ } | 1.94\hat{ } | 1.39\hat{ } | 1.56\hat{ } | 1.97\hat{ } |
| (ng · h/mL) | (133\hat{ }%- | (147\hat{ }%- | (157\hat{ }%- | (122%- | (136\hat{ }%- | (161\hat{ }%- |
| | 173\hat{ }%) | 192\hat{ }%) | 239\hat{ }%) | 159\hat{ }%) | 178\hat{ }%) | 241\hat{ }%) |
| AUC₀₋ₜ | 1.14 | 1.08 | 1.15 | 1.16 | 1.08 | 1.18 |
| (ng · h/mL) | (106%- | 105%- | (109%- | (109%- | (104%- | (113%- |
| | 122%) | 112%) | 120%) | 125%) | 111%) | 123%) |
| AUC_{0-inf} | 1.11 | 1.10 | 1.18 | 1.12 | 1.06 | 1.17 |
| (ng · h/mL) | (104%- | (106%- | (109%- | (106%- | (102%- | (109%- |
| | 117%) | 114%) | 127\hat{ }%) | 119%) | 110%) | 127*%) |
| Cₘₐₓ | 1.46\hat{ } | 1.54\hat{ } | 1.62\hat{ } | 1.46\hat{ } | 1.43\hat{ } | 1.59\hat{ } |
| (ng/mL) | (135\hat{ }%- | (143\hat{ }%- | (145\hat{ }%- | (136\hat{ }%- | (133\hat{ }%- | (143\hat{ }%- |
| | 157\hat{ }%) | 166\hat{ }%) | 182\hat{ }%) | 157\hat{ }%) | 154\hat{ }%) | 178*%) |
| Tₘₐₓ (hr) | 0.49\hat{ } | 0.45\hat{ } | 0.53\hat{ } | 0.56\hat{ } | 0.48\hat{ } | 0.51\hat{ } |
| (median) | (25\hat{ }%- | (26\hat{ }%- | (23\hat{ }%- | (32\hat{ }%- | (29\hat{ }%- | (22\hat{ }%- |
| | 73\hat{ }%) | 65\hat{ }%) | 83%) | 80%) | 68\hat{ }%) | 80%) |

\hat{ }values outside bioequivalence criteria for MOBIC tablets (80-125% MOBIC tablets reference)
# ratios are the geometric LS means ratio of the natural-log transformed PK parameter

And, Table 8 show the relative pAUC(0-t) for each of formulations (1)-(6) as compared to pAUC(0-t) for MOBIC tablets at the same time point in the same subject population. Notably, meloxicam exposure for the first 4 hours after administration, as measured by pAUC(0-4), was found to be about 40-200% higher for all formulations (1)-(6) and 75-180% higher for the nanosized formulations (1)-(3) as compared to MOBIC tablets, when compared within the same subject populations. Nanosized formulations showed particular advantages within the first 2 hours after dosing [pAUC(0-2)], with exposure being greater than 93-375% higher for all formulations (1)-(6) and 143-372% higher for the nanosized formulations (1)-(3) as compared to MOBIC tablets, when compared within the same subject populations. In each instance, formulations (1)-(6), while well tolerated, did not meet the bioequivalence criteria (see, FDA Guidance for Industry, “Bioavailability and Bioequivalence Studies for Orally Administered Drug Products—General Considerations”, March 2003) with regard to rate and extent of absorption under fasting conditions.

### Example 9. Food Effect Studies

A single-dose, randomized, four-period, four treatment, crossover study investigated the bioavailability of Meloxicam: Salicylic Acid (1:1) Cocrystal Tablets (3, nanosized), relative to MOBIC Tablets, 15 mg (Boehringer Ingelheim Pharmaceuticals, Inc) under fasting and fed conditions. Single-dose pharmacokinetics were characterized in 20 healthy, adult volunteers following administration of a single, oral 15 mg (1×15 mg) dose of study medication under fasting and fed conditions.

Fasting patients: After an overnight fast of at least 10 hours, one of the investigational treatments was administered to the volunteers on Day 1 of each study period. Subjects fasted until 4 hours after dosing. Standard low-fat meals were provided at least 4 hours after dosing and were provided approximately 10 hours after dosing and at other appropriate times thereafter.

Fed patients: After an overnight fast of at least 10 hours, subjects received a standardized high-fat breakfast which was consumed in its entirety within 30 minutes. The standard ‘high-fat’ breakfast consisted of 240 mL whole milk, 65 grams of chicken minced and sautéed in butter, 60 grams of bread toasted with butter, 115 grams of hash brown potato sautéed in butter, and 85 grams of eggs fried in butter. Substitution of the bacon with another animal protein was permissible as long as the substituted meat ensured that the total calories from protein (^{˜}150), carbohydrate (^{˜}250), and fat (^{˜}500-600) of the meal was maintained. Exactly 30 minutes after starting the standardized high-fat breakfast, one of the investigational treatments was administered to the volunteers on Day 1 of each study period. Subjects fasted until 5 hours after dosing. Standard low-fat meals were provided at least 5 hours after dosing and were provided approximately 10 hours after dosing and at other appropriate times thereafter.

In either instance, ambient temperature dosing water (240±10 mL) was provided in individual containers and the drug in unit-dose containers. All tablets were swallowed whole and not broken, chewed or crushed. Subjects generally remained seated, in an upright position for 5 hours following drug administration to ensure proper stomach emptying except for brief periods under close supervision.

Blood samples were collected prior to dosing and for 72 hours after each dosing period. There were at least 14 days between dosing times for the treatment periods. Four milliliter (1×4 mL) blood samples were collected in K₃EDTA tubes at pre-dose and the following times after dosing: 0.167, 0.333, 0.5, 0.75, 1, 1.25, 1.5, 1.75, 2, 2.5, 3, 3.5, 4, 5, 6, 7, 8, 10, 12, 16, 24, 36, 48, and 72 hours under fasting conditions and 0.25, 0.5, 0.75, 1, 1.5, 2, 2.5, 3, 3.5, 4, 4.5, 5, 5.5, 6, 6.5, 7, 8, 10, 12, 16, 24, 36, 48, and 72 hours under fed conditions. The pre-dose blood draw was collected no earlier than 120 minutes prior to dosing. Samples were handled and analyzed as described in Example 8.

TABLE 9
Comparative Food Effect Pharmacokinetic data (N = 19)

| | | (3, nanosized) | | | | MOBIC tablets | | | |
|---|---|---|---|---|---|---|---|---|---|
| Parameter | | Fasting (N = 19) | | Fed (N = 18) | | Fasting (N = 19) | | Fed (N = 18) | |
| AUC₀₋ₜ | (ng · h/mL)(CV %) | 53917.9 | (32.8%) | 53452.5 | (26.3%) | 49418.1 | (39.4%) | 60066.8 | (32.1%) |
| AUC_{0-inf} | (ng · h/mL)(CV %) | 71240.0 | (43.7%) | 66578.5 | (31.9%) | 65269.1 | (56.5%) | 77773.6 | (49.6%) |
| pAUC_{0-1 h} | (ng · h/mL)(CV %) | 1006.7 | (67.1%) | 167.8 | (92.3%) | 279.3 | (116.6%) | 100.8 | (183.9%) |
| pAUC_{0-2 h} | (ng · h/mL)(CV %) | 2768.6 | (51.1%) | 635.5 | (74.3%) | 970.0 | (82.6%) | 422.1 | (156.2%) |
| pAUC_{0-3 h} | (ng · h/mL)(CV %) | 4694.8 | (44.1%) | 1391.4 | (61.8%) | 2147.5 | (60.8%) | 1057.8 | (105.5%) |
| pAUC_{0-4 h} | (ng · h/mL)(CV %) | 6730.6 | (36.2%) | 2487.1 | (50.0%) | 3656.1 | (47.0%) | 2117.7 | (73.8%) |
| pAUC₀₋₆ | (ng · h/mL)(CV %) | 10330.1 | (26.6%) | 5724.9 | (32.2%) | 6498.4 | (33.7%) | 5300.6 | (43.4%) |
| Cₘₐₓ | (ng/mL) (CV %) | 2439.8 | (22.9%) | 2030.3 | (18.0%) | 1645.5 | (21.8%) | 1952.8 | (21.4%) |
| kₑₗ | (hr⁻¹) (CV %) | 0.0281 | (35.7%) | 0.0300 | (33.2%) | 0.0287 | (31.5%) | 0.0282 | (30.0%) |
| t_{1/2} | (hr) (CV %) | 28.12 | (39.2%) | 25.59 | (33.6%) | 27.35 | (48.1%) | 27.19 | (37.7%) |
| Tₘₐₓ | (hr) median (range) | 2.0 | (0.75-4.0) | 5.5 | (3.5-8.0) | 4.0 | (3.0-6.0) | 6.0 | (5.0-8.0) |

TABLE 10
Fed/Fasting Ratios (90% Confidence Intervals)

| | | Fed/Fasting | | | |
|---|---|---|---|---|---|
| Parameter | | (3, nanosized) | | MOBIC tablets | |
| AUC₀₋ₜ | (ng · h/mL) | 1.02 | (95.9%-109.5%) | 1.21 | (113.5%-129.0%) |
| pAUC_{0-1 h} | (ng · h/mL) | 0.13 | (7.7%-23.0%) | 0.20 | (11.9%-34.7%) |
| pAUC_{0-2 h} | (ng · h/mL) | 0.19 | (12.0%-30.7%) | 0.26 | (16.3%-40.9%) |
| pAUC_{0-3 h} | (ng · h/mL) | 0.26 | (18.3%-37.8%) | 0.38 | (26.3%-53.5%) |
| pAUC_{0-4 h} | (ng · h/mL) | 0.34 | (26.3%-44.8%) | 0.48 | (37.4%-62.7%) |
| pAUC₀₋₆ | (ng · h/mL) | 0.55 | (46.8%-63.7%) | 0.76 | (65.2%-88.2%) |
| AUC_{0-inf} | (ng · h/mL) | 0.99 | (93.0%-106.4%) | 1.17 | (109.7%-125.1%) |
| Cₘₐₓ | (ng/mL) | 0.85 | (79.2%-92.1%) | 1.18 | (110.0%-127.3%) |

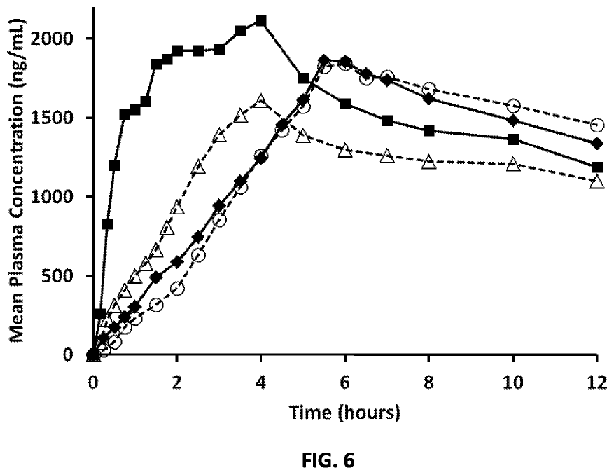

Mean plasma concentration of meloxicam for formulation (3) as compared to MOBIC tablets under fed and fasting conditions in the same subject cohort are shown in FIG. 6, and PK results are summarized in Tables 9-10.

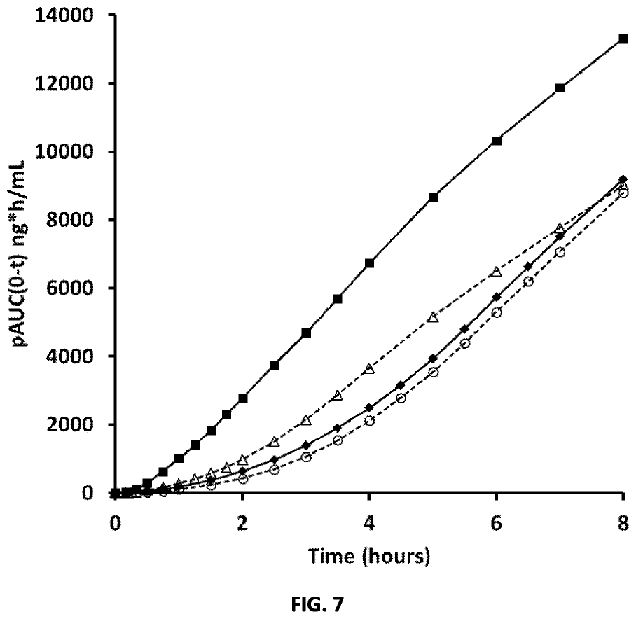

This study demonstrates that Mylan's formulation (3) tablets, 15 mg and Boehringer Ingelheim's MOBIC tablets, 15 mg behave differently following a single, oral 15 mg (1×15 mg) dose administered under fed conditions relative to fasting conditions. While formulation (3) tablets exhibited a 15% decrease in Cₘₐₓ with minimal change (<2%) seen for AUC₀₋ₜ and AUC_{0-inf} under high-fat fed conditions relative to fasting, an 18%, 21% and 17% increase was seen for Cₘₐₓ, AUC₀₋ₜ, and AUC_{0-inf}, respectively, for MOBIC tablets under fed conditions relative to fasting. The median Tₘₐₓ was delayed to a similar extent for both products, with formulation (3) showing an approximately 3.5 hour increase in Tₘₐₓ and MOBIC showing an approximately 2 hour increase in Tₘₐₓ under high-fat fed conditions. The fed/fasting ratios for pAUCs up to 6 hours post-dosing were similar for formulation (3) tablets and MOBIC tablets. In addition, the relative bioavailability of formulation (3) tablets compared to MOBIC tablets under fasting and fed conditions was also evaluated in this study. Under fasting conditions, formulation (3) tablets had a 48% higher Cₘₐₓ compared to MOBIC while AUC₀₋ₜ and AUC_{0-inf} met the bioequivalence criteria. Under fed conditions, Cₘₐₓ, AUC₀₋ₜ, and AUC_{0-inf}, all met bioequivalence criteria. The more rapid absorption of formulation (3) Tablets compared to MOBIC tablets under fasting conditions was evident by the approximately 4-, 3-, 2-, 2-, and 1.5-fold higher pAUC(0-1), pAUC(0-2), pAUC(0-3), pAUC(0-4), and pAUC(0-6), respectively. The pAUCs were also higher for formulation (3) tablets compared to MOBIC tablets under fed conditions, but to a lesser extent than under fasting conditions. Plots of pAUC(0-t) for formulation (3) under fed and fasting administration within the first eight hours after administration are provided in FIG. 7.

### Example 10. Summary of Pharmacokinetic Studies

TABLE 11
Ratios of pharmacokinetic parameters for Formulation (3)
versus MOBIC tablets (R) (90% confidence intervals)

| | | | |
|---|---|---|---|
| | Example 8 | Example 9 | Example 9 |
| Parameter | Fasting | Fasting | Fed |

| | | | | |
|---|---|---|---|---|
| pAUC_{0-1 h} | (ng · h/mL) | 6.27\hat{ } | 3.99\hat{ } | 2.61\hat{ } |
| | | (335\hat{ }%-1174\hat{ }%) | (236\hat{ }%-675\hat{ }%) | (149\hat{ }%-456\hat{ }%) |
| pAUC_{0-2 h} | (ng · h/mL) | 4.72\hat{ } | 3.20\hat{ } | 2.37\hat{ } |
| | | (268\hat{ }%-829\hat{ }%) | (203\hat{ }%-502%) | (147\hat{ }%-383\hat{ }%) |
| pAUC_{0-3 h} | (ng · h/mL) | 3.71\hat{ } | 2.32\hat{ } | 1.63\hat{ } |
| | | (229\hat{ }%-600\hat{ }%) | (164\hat{ }%-329\hat{ }%) | (112%-236\hat{ }%) |
| pAUC_{0-4 h} | (ng · h/mL) | 2.77\hat{ } | 1.91\hat{ } | 1.36\hat{ } |
| | | (191\hat{ }%-402\hat{ }%) | (148\hat{ }%-247\hat{ }%) | (103%-178\hat{ }%) |
| pAUC_{0-6 h} | (ng · h/mL) | 1.94\hat{ } | 1.62\hat{ } | 1.16 |
| | | (157\hat{ }%-239\hat{ }%) | (139\hat{ }%-187\hat{ }%) | (99%-136\hat{ }%) |
| AUC₀₋ₜ | (ng · h/mL) | 1.15 | 1.12 | 0.94 |
| | | (109%-120%) | (105%-119%) | (88%-101%) |
| AUC_{0-inf} | (ng · h/mL) | 1.18 | 1.12 | 0.95 |
| | | (109%-127\hat{ }%) | (105%-120%) | (89%-102%) |
| Cₘₐₓ | (ng/mL) | 1.62\hat{ } | 1.48\hat{ } | 1.07 |
| | | (145\hat{ }%-182\hat{ }%) | (138\hat{ }%-159\hat{ }%) | (99%-115%) |
| Tₘₐₓ | (hr) median | 0.53\hat{ } | 0.56\hat{ } | 0.91 |
| | | (23\hat{ }%-83%) | (44\hat{ }%-67\hat{ }%) | (83%-99%) |

\hat{ }values outside bioequivalence criteria for MOBIC tablets (80-125% reference)
# ratios are the geometric LS means ratio of the natural-log transformed PK parameter

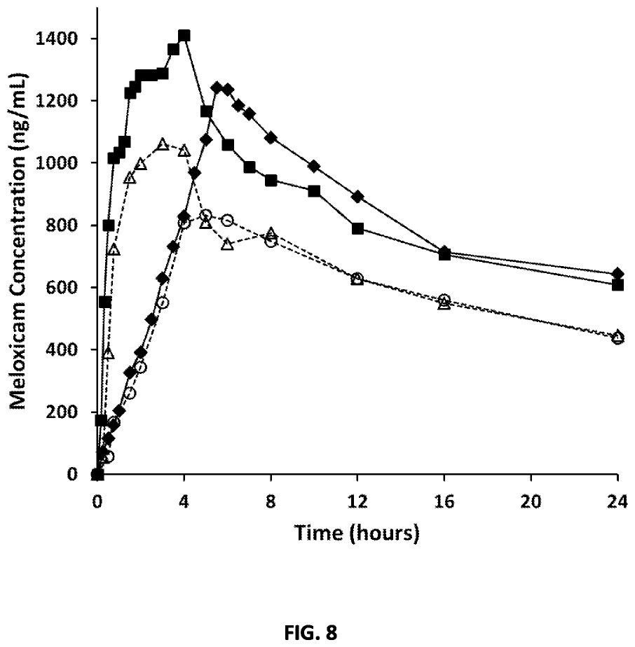

Table 11 shows a direct comparison for the measured pharmacokinetic parameter ratios from the studies of Examples 8 and 9 for formulation (3) versus MOBIC tablets under fed or fasting conditions, as relevant. It can be noted that exposure to meloxicam as measured by pAUC(0-t) is substantially higher for formulation (3) versus MOBIC tablets when administered in the fasting state. Further, the results herein were compared with the Summary Basis of Approval (SBOA) for VIVLODEX capsules (10 mg) [for NDA #207233, available at www.accessdata.fda.gov/drugsatfda_docs/nda/2015/207233Orig1s000TOC.cfm]. As shown in FIG. 8, on a dose adjusted basis (10 mg), the present formulation reaches a greater blood plasma concentration as compared to an equivalent dosage of VIVLODEX capsules.

It should be noted that while the Cₘₐₓ of meloxicam following dosing with 15 mg formulation (3) was approximately 50% greater than that of 15 mg MOBIC tablets, the overall exposure (AUC) was similar. The combination of similar overall exposure with the anticipated acute use of formulation (3) means that use of 15 mg formulation (3) is considered safe, especially as ANJESO (IV meloxicam) 30 mg has a reported Cₘₐₓ of 7972.5 ng/mL and an AUC_{inf} of 121437.6 ng·hr/mL.

### Example 11. Clinical Study for the Treatment of Acute Pain

Based on the demonstration that meloxicam is effective for the treatment of chronic pain, such as osteoarthritis, at doses of 7.5 mg/day (e.g., MOBIC tablets) and that this effect is mediated by inhibition of cyclooxygenase, a concentration of approximately 1090 ng/mL, which is similar to the steady state concentration of 7.5 mg meloxicam, could be considered sufficient for mediating an analgesic response. Ex vivo assays performed on samples taken from subjects receiving 7.5 mg meloxicam have demonstrated an 83% inhibition of COX-2 (see, Bain et al., Limitation of the in vitro whole blood assay for predicting the COX selectivity of NSAIDs in clinical use. Br. J. Clin. Pharmacol., 2002. 53(3): p. 255-65).

As described in the single dose Phase 1 study of Example 8, micronized formulation (6), and nanosized formulation (3) of meloxicam: salicylic acid (1:1) co-crystals (“MECC-SA”) demonstrated rapid absorption of meloxicam versus MOBIC tablets but in a less invasive manner than the IM and IV formulations that have demonstrated efficacy in the treatment of acute pain. On the basis of this study, it is estimated that a 15 mg dose of a meloxicam-salicylic acid formulation would exceed 1090 ng/mL within 45 minutes of dosing, therefore acute analgesia could be anticipated at or around this time (Table 12).

TABLE 12
Example 8, 15 mg

| | Formulation | Formulation | MOBIC tablets |
|---|---|---|---|
| Parameter | (6) | (3) | 15 mg |
| Time to achieve | 0.75 (0.33-3.0) | 1.25 (0.17-5.0) | 2.50 (1.25-8.0) |
| 1090 ng/mL | | | |
| (hours), | | | |
| median (range) | | | |

The Phase 1 study of Example 9, studied the effect of a high fat meal on the PK of meloxicam, and demonstrated a food effect with a delay to the absorption of meloxicam from formulation (3), a reduced Cₘₐₓ and later Tₘₐₓ in the presence of food, suggesting that a preferred situation for achieving acute analgesia would be in the absence of a high-fat meal. On the basis of Example 9, it is estimated that a 15 mg dose of MECC-SA (in the fasted state) would exceed 1090 ng/mL within 30 minutes of dosing, therefore acute analgesia could be anticipated at or around this time (Table 13).

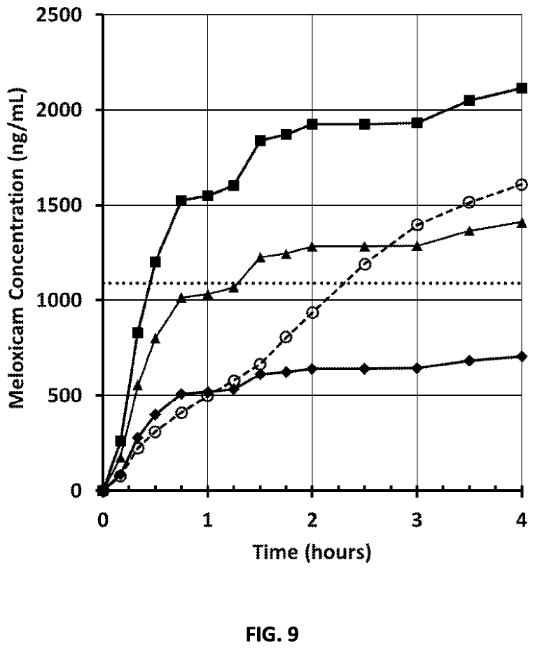

As a visualization, the mean plasma meloxicam concentration vs time data for formulation (3) as determined in Example 9 was dose adjusted assuming pharmacokinetic linearity across 5-15 mg strengths to generate the plot of FIG. 9. Therein, the mean plasma meloxicam concentration achievable from 5, 10, and 15 mg dosages of formulation (3, MECC-SA) are compared to 15 mg MOBIC tablets. A therapeutic blood concentration for analgesia is shown by the dotted line in FIG. 9 at 1090 ng/mL. As shown in FIG. 9, 15 mg of MECC-SA formulation (3) would achieve this concentration within approximately 30 minutes and a 10 mg dose-proportional formulation of the same co-crystal formulation would achieve this concentration within approximately 1.25-1.5 hours; in contrast, MOBIC tablets required about 2.5 hours. This rapid uptake can enable significantly faster time to therapeutic meloxicam concentrations and applications to the treatment of acute pain, where rapid time to analgesia are necessary and are provided for the first time by the formulations in this application.

TABLE 13
Example 9, 15 mg

| | Fasting | | Fed | |
|---|---|---|---|---|
| | Formulation | MOBIC | Formulation | MOBIC |
| Parameter | (3) | tablets | (3) | tablets |
| Time to achieve | 0.50 (0.17-4.0) | 2.5 (0.33-4.0) | 3.75 (1.5-6.5) | 4.25 (0.75-6.0) |
| 1090 ng/mL | | | | |
| (hours), | | | | |
| median (range) | | | | |

Predicted exposure for BID dosing of MECC-SA are summarized in Table 14, based on the fasted data from the study of Example 9. The predicted exposure for 10 and 15 mg BID (i.e., two doses over 24 hours) MECC-SA will exceed that of a single dose of 15 mg MECC-SA formulation (3) or MOBIC tablets, however, the overall exposure and Cₘₐₓ (after the second dose) would be similar to or less than that of ANJESO and therefore for use in the treatment of acute pain it can be anticipated that MECC-SA will be well tolerated.

TABLE 14
Predicted Meloxicam Exposure After Single Doses, Once and Twice Daily Dosing vs. MOBIC and ANJESO

| | | Parameter | | | | | | | | | |
|---|---|---|---|---|---|---|---|---|---|---|---|
| | | Formulation (3) | | | | | | MOBIC | | ANJESO | |
| | | (fasting) | | | | | | (fasting) | | Study 1 | Study 2 |
| | | Dosage | | | | | | | | | |
| | | 15 mg | | 15 mg | | 10 mg | | 15 mg | | | |
| | | Dosing frequency | | | | | | | | | |
| | | QD | | BID | | BID | | QD | | 30 mg | 30 mg |
| AUC_{0-inf} | (ng · hr/mL) | 71240.0 | (43.7%) | 123229.7 | (60.7%) | 82153.1 | (60.7%) | 65269.1 | (56.5%) | 107508.7 | 121437.6 |
| Cₘₐₓ | (ng/mL) | 2439.8 | (22.9%) | 3550.7 | (20.2%) | 2367.1 | (20.2%) | 1645.5 | (21.8%) | 5642.9 | 7927.5 |
| Tₘₐₓ | (hr) median | 2.0 | (0.75-4.0) | 14.0 | (12.75-16.0)* | 14.0 | (12.75-16.0)* | 4.0 | (3.0-6.0) | 0.12 | — |
| | (range) | | | | | | | | | | |

*Time calculated from first dose, i.e., 2.0 hr after second dose.
All parameters are mean except Tₘₐₓ (median).

Study Design. This will be a randomized, double-blind, placebo-controlled, parallel group, dose-ranging study, randomizing approximately 110 male or female subjects that have had third molar dental surgery. This study will specifically identify if MECC-SA has efficacy in a model of acute post-operative pain and will enable the identification of the most appropriate dose and regime of MECC-SA to continue in development Approximately 110 subjects will be randomized to give approximately N=100 evaluable subjects for the primary endpoint (N=20 per treatment arm). Subjects will be randomized to receive MECC-SA (10 mg QD, 15 mg QD, 10 mg BID or 15 mg BID) or matching placebo in a 1:1:1:1:1 ratio.

The study design is consistent with research design recommendations of the IMMPACT (Initiative on Methods, Measurement, and Pain Assessment in Clinical Trials) group for trials involving subjects with short-duration acute pain (see Cooper et al., Research design considerations for single-dose analgesic clinical trials in acute pain: IMMPACT recommendations. Pain, 2016. 157(2): p. 288-301).

Subjects will attend screening at Visit 1, to occur within 30 days prior to the planned dental surgery. The dental surgery (extraction of ≥2× third molars, at least 1 of which involves partial or complete mandibular bony extraction) will be performed on Visit 2 (Day 1). The procedure will be performed using appropriate local anesthesia and sedation (performed using nitrous oxide and a local injection of lidocaine with epinephrine). Subjects will also receive prophylactic antibiotics post surgically.

Subjects will be eligible for randomization based on Pain Intensity (PI) and Categorical Pain Rating (CPR) following surgery. Pain Intensity (PI) will be assessed using a 0-10 point numeric pain rating scale (NPRS) where 0 is no pain and 10 is the worst pain imaginable) during the 5 hours following surgery (see, Breivik et al., Assessment of pain. Br. J. Anaesth., 2008. 101(1): p. 17-24). Categorical Pain Rating will be assessed using a 4-point categorical pain rating scale (none, mild, moderate, severe). Subjects scoring an NPRS ≥5 and moderate or severe pain on CPR within 5 hours following surgery will be eligible for randomization. Assessments will be made following surgery until a baseline pain is achieved.

Subjects will fast from midnight prior to the surgery and will be allowed to eat food after 2 hours post-dose. Subjects should not consume any water or other drinks for 2 hours prior to the surgery.

Eligible subjects will receive study drug on Day 1. MECC-SA will be provided as 10 and 15 mg tablets (and matching placebo). MECC-SA, 15 mg (meloxicam base) will be dosed via tablets of formulation (3), while 10 mg (meloxicam base) MECC-SA will be dosed via dose-proportional tablets. Placebo is a tablet identical to the corresponding dose of active study drug. To maintain the blind, two tablets of study drug or placebo will be administered on one day, Visit 2 (Day 1). To maintain the blind, regarding QD and BID dosing, subjects assigned to QD treatment arms will receive placebo at the second dose. Study drugs will be administered following randomization and approximately 12 hours following the first dose.

Study drug (two tablets containing MECC-SA or matching placebo) will be administered in the clinic at each dosing time. Subjects should take the study drugs with about 240 mL of water at ambient temperature in each period under the supervision of trained study personnel. Study drugs must be swallowed whole and must not be chewed, crushed or divided.

Rescue medication of immediate release hydrocodone/acetaminophen (5/500 mg) will be allowed to treat breakthrough pain, subjects will be encouraged to refrain from rescue medication until at least 1-hour post-dose. A dose of rescue medication will be allowed as needed up to once every two hours. A maximum of 6 tablets (equivalent to 3000 mg acetaminophen) will be allowed; if a subject requires greater than this the subject should be withdrawn from the study.

Data Collection (Day 1). Pain Intensity (0-10-point NPRS) will be measured at 15, 30, and 45 minutes and 1, 1.5, 2, 3, 4, 5, 6, 7, 8, 12, 14, and 16 hours after the first dose of study medication and immediately before any rescue medication and/or at the time of early termination. Assessments will also be made immediately following the time of first relief and time of meaningful relief.

Pain Relief Assessments (5-point scale—none=0, slight=1, moderate=2, good or a lot=3, and complete=4) will be measured at 15, 30, and 45 minutes and 1, 1.5, 2, 3, 4, 5, 6, 7, 8, 12, 14, and 16 hours after the first dose of study medication and immediately before any rescue medication and/or at the time of early termination. Assessments will also be made immediately following the time of first relief and time of meaningful relief.

Time of first perceptible relief will be recorded (double-stopwatch method)—time the first stopwatch is stopped. Defined as post-dose time at which the subject first begins to feel pain relief. Time of meaningful relief will be recorded (double-stopwatch method)—time the second stopwatch is stopped. Defined as the post-dose time at which the subject begins to feel meaningful pain relief.

PK (pharmacokinetic) blood samples for analysis of meloxicam will be collected at pre-dose, 15, 30, and 45 minutes and 1, 1.5, 2, 3, 4, 5, 6, 7, 8, 12, 14, and 16 hours after the first dose of study medication. Where PK samples and pain assessments occur at the same time, the pain assessment should be made prior to the PK sample. Samples will be analyzed using validated analytical methods in compliance with standard operating procedures.

Adverse events will be assessed throughout the day.

Use of rescue medication while the subject is in the clinic will be recorded, including the dose and time of administration. Pain Intensity (PI) will be measured immediately before any rescue medication is administered.

Data Collection (Day 2)

Adverse events should be assessed throughout the day and any use of rescue medication should be recorded throughout the day.

Pain Intensity (0-10-point NPRS) measured at 20 and 24 hours after the first dose (Day 1) of study medication and immediately before any rescue medication.

Patient Global Assessment (PGA) of pain control will be made at 24 hours after the first dose and will be rated on a 5-point scale, ranging from 0 to 4, where 0-poor, 1-fair, 2 good, 3-very good, or 4-excellent.

A PK sample will be collected at 20 and 24 hours after the first dose of study medication.

Primary Efficacy Endpoint(s). The primary analysis of the primary efficacy endpoint will be based on the intention-to-treat (ITT) population. The primary efficacy endpoint is the overall (summed) pain intensity difference over 0-24 hours (SPID 0-24). A 3-parameter Eₘₐₓ model will be fitted to the SPID 0-24 standardized means (observed mean for each dose and placebo divided by the pooled standard deviation) for placebo and 10 mg and 15 mg MECC-SA. Standardized effect sizes will be estimated from the Eₘₐₓ model together with their 95% confidence intervals and p-values. Baseline pain intensity score (PI) will be used as a covariate in the Eₘₐₓ model. The numerical dose values will be 10, 15, 20, 30 and 0 for 10 mg QD, 15 mg QD, 10 mg BID, 15 mg BID and placebo, respectively.

Secondary Endpoints

Secondary SPID Endpoints and TOTPAR. The SPID endpoints for the time intervals 0-4, 0-8, 0-12 and 12-24 hours will be analyzed using the Eₘₐₓ/ANCOVA model used for the analysis of the primary endpoint. The TOTPAR (total pain relief) endpoints for the time intervals 0-4, 0-8, 0-12, 12-24 and 0-24 hours will be analyzed using the Eₘₐₓ/ANCOVA model used for the analysis of the primary endpoint.

Pain Intensity Differences (PID) Over Time. PID scores over time will be analyzed using a longitudinal mixed model for repeated measures (MMRM) with fixed effects for treatment, study site, time, treatment by time interaction and baseline PI score. Subject will be fitted as a random effect. Variance estimation will be based on an unstructured covariance matrix. Missing data will be assumed to be Missing At Random (MAR). Least squares means (LS means) will be plotted by time to show the pain-relieving efficacy of both MECC-SA and placebo over 24 hours.

Time to Event Endpoints. The following analysis methods apply to the time-to-event endpoints listed:
Time to perceptible pain relief (PPR).
Time to meaningful pain relief (MPR).
Elapsed time from the start of study medication to first rescue medication administration.

Time to event endpoints will be analyzed and plotted using Kaplan-Meier methodology and differences between MECC-SA and placebo evaluated using the log-rank test. The median time to event will be calculated and presented.

Responder Endpoints. The following analysis methods apply to the responder endpoints listed:
Proportion of subjects with overall pain reductions from baseline of 30% and 50% within 4 hours following the first dose based on the PI scores. Both these analyses will be presented for both methods of handling rescue medication use (W2LOCF and W4LOCF approaches).
Proportion of subjects using rescue medication during 0-24 hours.
Patient's Global Assessment (PGA) of pain control for 0-24 hours after the initial dose scored on the 5-point scale will be dichotomized and the percentage of subjects reporting a PGA score of 2 (good) or better defined as a responder.
   The number and percentage of responders in each treatment group will be summarized. A comparison between MECC-SA and placebo will be evaluated with the odds ratio (OR) and corresponding 95% confidence interval from a logistic regression model with treatment group and baseline PI as a covariate.

Frequency of Rescue Medication Use. The frequency of rescue medication use will be summarized and graphically presented for the 0-24 hours period after the first dose of study treatment.

Pharmacokinetics. Samples for PK determination of drug concentrations of meloxicam, in plasma will be determined by a bioanalytical laboratory using validated bioanalytical methods. The plasma concentration data for meloxicam will be listed, summarized on the basis of time intervals, and plotted using a scatter plot with time relative to the preceding meloxicam dosing time. Summary statistics (mean, standard deviation, minimum, maximum, number of subjects, and coefficient of variation) will be calculated for plasma concentrations for each time interval and by treatment group. Population PK (PPK) analysis of meloxicam concentrations will be performed using modeling. The PPK analysis will be conducted and reported separately. Details of the PPK model building will be described in a separate PPK analysis plan. Dose-response relationship may be established in this study, else exploratory exposure-response analysis may be conducted using individual predictions of exposure from the PPK analysis in this study.

### Example 12. Clinical Study for Treatment of Post-Surgical Dental Pain

A randomized, double-blind, placebo-controlled, parallel group, dose-response clinical study of meloxicam co-crystal in the treatment of post-surgical pain was performed following the study design outlined in Example 11. The study was designed to identify if MECC-SA has efficacy in a model of acute post-operative pain, as well as the most appropriate dose and treatment regime for MECC-SA. For this study, 112 subjects randomized and dosed either received MECC-SA (nanosized formulation (3) of meloxicam: salicylic acid (1:1) co-crystals) as 10 mg QD, 15 mg QD, 10 mg BID or 15 mg BID, or received matching placebo in an approximately 1:1:1:1:1 ratio. 110 subjects completed the study.

The primary endpoint for the study was SPID 0-24 (Summed Pain Intensity Difference over 24h). Table 15 summarizes primary endpoint values for the MECC-SA 10 mg QD, MECC-SA 15 mg QD, MECC-SA 10 mg BID, MECC-SA 15 mg BID, and placebo study arms.

TABLE 15
Summary of SPIDs: mITT W4LOCF*
Population: Modified Intent-to-treat Analysis Set

| | MECC-SA | | MECC-SA | | MECC-SA | | MECC-SA | | | |
|---|---|---|---|---|---|---|---|---|---|---|
| Interval | 10 mg QD | | 15 mg QD | | 10 mg BID | | 15 mg BID | | Placebo | |
| Statistic | (N = 21) | | (N = 24) | | (N = 23) | | (N = 23) | | (N = 21) | |
| SPID₀₋₂₄ | | | | | | | | | | |
| n | 21 | | 24 | | 23 | | 23 | | 21 | |
| Mean | 101.0 | | 102.4 | | 102.0 | | 104.3 | | 66.4 | |
| SD | 35.93 | | 44.90 | | 34.21 | | 39.63 | | 39.97 | |
| Median | 90.6 | (57, 182) | 96.9 | (14, 187) | 91.8 | (46, 173) | 107.9 | (20, 153) | 72.4 | (−21, 133) |
| (Min, Max) | | | | | | | | | | |
| Emax Estimates | | | | | | | | | | |
| Mean (SE) | 98.9 | (6.48) | 101.9 | (3.91) | 103.5 | (4.00) | 105.3 | (5.98) | 66.3 | (7.71) |

| 95% CI | (86.1, 111.8) | | (94.1, 109.6) | | (95.6, 111.4) | | (93.5, 117.2) | | (51.0, 81.6) |
|---|---|---|---|---|---|---|---|---|---|
| Emax Estimated | | | | | | | | | |
| Difference vs. | | | | | | | | | |
| Placebo | | | | | | | | | |
| Mean (SE) | 32.7 | (10.02) | 35.6 | (8.67) | 37.2 | (8.72) | 39.1 | (9.77) | n/a |

| | | | | | |
|---|---|---|---|---|---|
| 95% CI | (12.8, 52.5) | (18.4, 52.8) | (20.0, 54.5) | (19.7, 58.4) | n/a |
| p-value | 0.001 | <0.001 | <0.001 | <0.001 | n/a |

*W4LOCF utilizes “pain right now” just prior to rescue medication use and censors subsequent pain intensity values for 4 hours when calculating SPIDs.

The data in Table 15 show that all active treatment groups have a statistically significant difference versus placebo, demonstrating that MECC-SA displayed acute analgesia over a 24 h period following the first dose of study treatment. The mean data from each treatment arm is reported in the table and these data are fitted into an Emax model to describe the dose response curve for the drug. This allows for a mean Emax difference from placebo, which is reported in the lower part of the table.

Table 16 summarizes the time to perceptible relief, which demonstrates when a subject starts to feel any pain relief after taking a study treatment. Time to perceptible relief was measured using a stopwatch, which was started when the subject first received the study treatment and stopped when the subject first felt any perceptible pain relief; if a subject received rescue medication prior to recording perceptible pain relief, the stopwatch was removed and their data was censored. Data from the QD and BID treatment arms were pooled to better understand the data. This was appropriate since the time to perceptible pain endpoint is typically observed in the first few hours post-dose.

TABLE 16
Summary of Time to Perceptible Relief
Combined Doses, Results through 12 hours.
Population: Modified Intent-to-treat Analysis Set

| | MECC-SA, | | MECC-SA, | | | |
|---|---|---|---|---|---|---|
| | 10 mg | | 15 mg | | Placebo | |
| | (N = 44) | | (N = 47) | | (N = 21) | |
| Subjects with any | | | | | | |
| Perceptible Relief | | | | | | |
| n (%) | 31 | (70.5) | 41 | (87.2) | 10 | (47.6) |
| Time to first event (hours)** | | | | | | |
| 75^{th} percentile (95% CI) | NA | (1.1, NA) | 1.2 | (0.8, 2.1) | NA | (NA, NA) |
| Median (95% CI) | 0.7 | (0.5, 1.7) | 0.6 | (0.5, 0.9) | NA | (0.3, NA) |
| 25^{th} percentile (95% CI) | 0.3 | (0.3, 0.5) | 0.3 | (0.2, 0.5) | 0.3 | (0.1, 0.8) |

| | | | |
|---|---|---|---|
| p-value (Pooled vs Placebo) | | | 0.026 |
| p-value (Pairwise vs Placebo) | 0.336 | 0.007 | |

Note:
Subjects were censored at 12 hours if they did not report relief.
**Percentiles and comparisons are based on Kaplan-Meier method and log-rank test. Values marked NA (not applicable) have insufficient counts of events to be estimable from the K-M methodology.

The data in Table 16 show that 70.5% and 87.2% of subjects receiving the 10 mg and 15 mg MECC-SA treatments, respectively, reported perceptible pain relief, whereas fewer than 50% of placebo subjects reported such relief. The median time to reporting was 0.7 hours for the 10 mg MECC-SA treatment arm and 0.6 hours for the 15 mg MECC-SA treatment arm.

Tables 17-20 summarize pharmacokinetic data for each of the four treatment arms.

TABLE 17
Plasma Meloxicam Concentration (ng/mL) -
primary analysis, 10 mg QD treatment arm

| | PK Parameters | | | | | |
|---|---|---|---|---|---|---|
| | Cₘₐₓ | Tₘₐₓ | T₁₀₉₀ | AUC_{0-2 hr} | AUC_{0-4 hr} | AUC_{0-8 hr} |
| | (ng/mL) | (hr) | (hr) | (ng*hr/mL) | (ng*hr/mL) | (ng*hr/mL) |
| N | 21 | 21 | 16 | 21 | 21 | 21 |
| Mean | 1243 | 3.82 | 2.857 | 910.470 | 2786.979 | 6176.383 |
| STD | 274 | 3.10 | 2.053 | 548.824 | 942.613 | 1535.112 |
| CV | 22 | 81.1 | 71.848 | 60.279 | 33.822 | 24.855 |
| Min | 797 | 0.52 | 0.517 | 79.169 | 741.294 | 3875.049 |
| Median | 1191 | 2.98 | 2.483 | 916.940 | 2889.052 | 5891.448 |
| Max | 1818 | 14.2 | 8.000 | 2038.961 | 4249.519 | 9925.012 |
| Geomean | 1215 | 2.96 | 2.271 | 706.207 | 2587.783 | 5997.486 |

| | PK Parameters | | |
|---|---|---|---|
| | AUC_{0-12 hr} | AUC_{0-24 hr} | AUC_{12-24 hr} |
| | (ng*hr/mL) | (ng*hr/mL) | (ng*hr/mL) |
| N | 21 | 21 | 21 |
| Mean | 9186.827 | 16125.093 | 6938.266 |
| STD | 1995.868 | 3365.045 | 1887.253 |
| CV | 21.725 | 20.868 | 27.201 |
| Min | 6277.329 | 11156.478 | 3576.017 |
| Median | 8673.398 | 15232.708 | 6731.987 |
| Max | 14345.574 | 23624.585 | 10734.487 |
| Geomean | 8992.136 | 15801.967 | 6684.777 |

TABLE 18
Plasma Meloxicam Concentration (ng/mL) -
primary analysis, 15 mg QD treatment arm

| | PK Parameters | | | | | |
|---|---|---|---|---|---|---|
| | Cₘₐₓ | Tₘₐₓ | T₁₀₉₀ | AUC_{0-2 hr} | AUC_{0-4 hr} | AUC_{0-8 hr} |
| | (ng/mL) | (hr) | (hr) | (ng*hr/mL) | (ng*hr/mL) | (ng*hr/mL) |
| N | 24 | 24 | 24 | 24 | 24 | 24 |
| Mean | 2297 | 2.89 | 1.996 | 2231.950 | 5668.607 | 11340.460 |
| STD | 769 | 1.88 | 1.599 | 1578.506 | 2594.126 | 3626.534 |
| CV | 33 | 65.2 | 80.101 | 70.723 | 45.763 | 31.979 |
| Min | 1172 | 0.52 | 0.233 | 338.953 | 1049.111 | 5563.906 |
| Median | 2148 | 2.43 | 2.000 | 1530.545 | 5479.446 | 11684.134 |
| Max | 4735 | 8.05 | 6.033 | 6516.468 | 12939.937 | 22211.531 |
| Geomean | 2189 | 2.39 | 1.339 | 1739.956 | 5056.196 | 10834.768 |

| | PK Parameters | | |
|---|---|---|---|
| | AUC_{0-12 hr} | AUC_{0-24 hr} | AUC_{12-24 hr} |
| | (ng*hr/mL) | (ng*hr/mL) | (ng*hr/mL) |
| N | 24 | 24 | 24 |
| Mean | 16140.781 | 27133.376 | 10992.595 |
| STD | 4715.320 | 8126.789 | 3798.747 |
| CV | 29.214 | 29.951 | 34.557 |
| Min | 9224.060 | 16983.401 | 6725.842 |
| Median | 15861.810 | 25433.415 | 9553.516 |
| Max | 31697.820 | 55623.578 | 23925.758 |
| Geomean | 15577.922 | 26187.759 | 10496.809 |

TABLE 19
Plasma Meloxicam Concentration (ng/mL) -
primary analysis, 10 mg BID treatment arm

| | PK Parameters | | | | | |
|---|---|---|---|---|---|---|
| | Cₘₐₓ | Tₘₐₓ | T₁₀₉₀ | AUC_{0-2 hr} | AUC_{0-4 hr} | AUC_{0-8 hr} |
| | (ng/mL) | (hr) | (hr) | (ng*hr/mL) | (ng*hr/mL) | (ng*hr/mL) |
| N | 23 | 23 | 23 | 23 | 23 | 23 |
| Mean | 1933 | 14.3 | 5.698 | 1047.573 | 3196.038 | 6670.341 |
| STD | 487 | 5.40 | 5.708 | 575.903 | 1101.425 | 1737.684 |
| CV | 25 | 37.7 | 100.170 | 54.975 | 34.462 | 26.051 |
| Min | 1144 | 1.97 | 0.967 | 21.746 | 1163.862 | 2581.859 |
| Median | 1902 | 15.7 | 2.933 | 1046.337 | 3124.036 | 6509.921 |
| Max | 2929 | 24.3 | 16.033 | 2409.614 | 4755.672 | 10488.848 |
| Geomean | 1873 | 12.5 | 3.670 | 810.946 | 2986.595 | 6425.225 |

| | PK Parameters | | |
|---|---|---|---|
| | AUC_{0-12 hr} | AUC_{0-24 hr} | AUC_{12-24 hr} |
| | (ng*hr/mL) | (ng*hr/mL) | (ng*hr/mL) |
| N | 23 | 23 | 23 |
| Mean | 9804.104 | 27568.328 | 17764.224 |
| STD | 2481.605 | 6203.757 | 4707.898 |
| CV | 25.312 | 22.503 | 26.502 |
| Min | 4531.719 | 16990.593 | 10823.747 |
| Median | 9728.565 | 28790.392 | 17958.559 |
| Max | 15774.373 | 43989.570 | 28215.197 |
| Geomean | 9483.228 | 26893.382 | 17180.937 |

TABLE 20
Plasma Meloxicam Concentration (ng/mL) -
primary analysis, 15 mg BID treatment arm

| | PK Parameters | | | | | |
|---|---|---|---|---|---|---|
| | Cₘₐₓ | Tₘₐₓ | T₁₀₉₀ | AUC_{0-2 hr} | AUC_{0-4 hr} | AUC_{0-8 hr} |
| | (ng/mL) | (hr) | (hr) | (ng*hr/mL) | (ng*hr/mL) | (ng*hr/mL) |
| N | 23 | 23 | 23 | 23 | 23 | 23 |
| Mean | 2922 | 13.7 | 2.059 | 1775.730 | 4903.162 | 10407.122 |
| STD | 827 | 5.07 | 1.347 | 1473.561 | 2317.036 | 3277.198 |
| CV | 28 | 37.1 | 65.415 | 82.983 | 47.256 | 31.490 |
| Min | 1362 | 0.78 | 0.517 | 156.637 | 1306.553 | 5621.395 |
| Median | 2768 | 14.1 | 1.583 | 1501.221 | 4829.374 | 10485.920 |
| Max | 4528 | 20.1 | 5.033 | 5691.623 | 9922.750 | 16770.152 |
| Geomean | 2808 | 11.5 | 1.645 | 1196.036 | 4312.514 | 9909.151 |

| | PK Parameters | | |
|---|---|---|---|
| | AUC_{0-12 hr} | AUC_{0-24 hr} | AUC_{12-24 hr} |
| | (ng*hr/mL) | (ng*hr/mL) | (ng*hr/mL) |
| N | 23 | 23 | 23 |
| Mean | 15158.929 | 41584.443 | 26425.514 |
| STD | 4247.275 | 11398.384 | 7613.313 |
| CV | 28.018 | 27.410 | 28.810 |
| Min | 8005.203 | 19077.707 | 11072.505 |
| Median | 14610.126 | 39197.153 | 25642.566 |
| Max | 23649.172 | 67741.072 | 44595.464 |
| Geomean | 14596.946 | 40091.327 | 25350.487 |

Plasma meloxicam PK parameters for the placebo arm were either zero (Cₘₐₓ and all AUC values) or n/a (not applicable, for Tₘₐₓ and T1090).

It is to be understood that the description of the present disclosure has been simplified to illustrate elements that are relevant for a clear understanding of the invention, while eliminating, for purposes of clarity, other elements that may be well known. In view of the above description, accompanying drawing figures, and the examples, one of ordinary skill in the art will be able to practice the instant description without undue experimentation. The foregoing will be better understood with reference to the following examples that detail certain procedures for the preparation of molecules and compositions described herein. All references made to the examples should not be considered exhaustive, nor limiting, but merely illustrative of only a few of the many aspects and embodiments contemplated by the present disclosure.

1. A method for treating acute pain comprising administering to a person in need of such treatment an oral solid pharmaceutical composition comprising a meloxicam co-crystal.

2. The method of claim 1, wherein the acute pain is post-surgical pain.

3. The method of claim 1, wherein the meloxicam co-crystal is a meloxicam nano-cocrystal.

4. The method of claim 1, wherein the pain is post-surgical pain and the oral solid composition provides a reduction of rescue medication use as compared to patients receiving placebo tablets as measured in a post-surgical dental pain study.

5. The method of claim 1, wherein the oral solid composition comprises an amount of meloxicam co-crystal equivalent to between about 1 mg and 60 mg meloxicam base.

6. The method of claim 1, wherein the oral solid composition provides a reduced mean time or reduced median time of first perceptible relief, and/or a reduced mean time or reduced median time of meaningful relief as compared to placebo tablets as measured in a post-surgical dental pain study.

7. The method of claim 1, wherein the administering is once daily administration.

8. The method of claim 1, wherein the administering is twice daily administration.

9. The method of claim 1, wherein the person in need of such treatment is a person diagnosed with an opioid use disorder.

10. The method of claim 1, wherein the meloxicam co-crystal is selected from the group consisting of meloxicam: succinic acid (2:1); meloxicam: aspirin (1:1), meloxicam: xinafoic acid (1:1), meloxicam: salicylic acid (1:1), and meloxicam: maleic acid (1:1).

11. The method of claim 10, wherein the meloxicam co-crystal is meloxicam: salicylic acid (1:1).

12. The method of claim 1, wherein the oral solid composition provides a therapeutic blood plasma concentration of meloxicam in less than about 3 hours as measured in a post-surgical dental pain study or a single-dose pharmacokinetic study.

13. The method of claim 12, wherein the oral solid composition provides the therapeutic blood plasma concentration of meloxicam in between about 0.5 hour and 3 hours.

14. The method of claim 13, wherein the oral solid composition provides the therapeutic blood plasma concentration of meloxicam in less than about 2.5 hours.

15. The method of claim 1, wherein the oral solid pharmaceutical composition contains an amount of meloxicam co-crystal equivalent to about 10 mg or about 15 mg meloxicam base and provides a blood plasma concentration of meloxicam of between 500-2500 ng/mL, or a bioequivalent thereof, in less than about 2 hours, as measured in a single-dose pharmacokinetic study or a post-surgical dental pain study.

16. The method of claim 1, wherein the oral solid pharmaceutical composition contains an amount of meloxicam co-crystal equivalent to about 10 mg or about 15 mg meloxicam base and provides a blood plasma concentration of meloxicam of:
(a) between 1000-2500 ng/mL, or a bioequivalent thereof, in less than about 2 hours; and
(b) between 500-1500 ng/mL, or a bioequivalent thereof, in less than about 0.5 hour;
as measured in a single-dose pharmacokinetic study or a post-surgical dental pain study.

17. The method of claim 15, wherein the oral solid pharmaceutical composition contains an amount of meloxicam co-crystal equivalent to about 15 mg meloxicam base.

18. The method of claim 17, wherein the oral solid composition provides a blood plasma concentration of meloxicam of:
(a) between 1000-2500 ng/mL, or a bioequivalent thereof, in less than about 2 hours; and
(b) between 500-1500 ng/mL, or a bioequivalent thereof, in less than about 0.5 hour;
as measured in a single-dose pharmacokinetic study or a post-surgical dental pain study.

19. The method of claim 15, wherein the oral solid pharmaceutical composition contains an amount of meloxicam co-crystal equivalent to about 10 mg meloxicam base and provides a blood plasma concentration of between 500-1500 ng/mL, or a bioequivalent thereof, in less than about 2 hours, as measured in a single-dose pharmacokinetic study or a post-surgical dental pain study.

20. The method of claim 19, wherein the oral solid pharmaceutical composition provides a blood plasma concentration of meloxicam of:
(a) between 1000-1500 ng/mL, or a bioequivalent thereof, in less than about 2 hours; and
(b) between 500-1000 ng/mL, or a bioequivalent thereof, in less than about 0.5 hour;
as measured in a single-dose pharmacokinetic study or a post-surgical dental pain study.
